SEQUENCE LISTING (2 sequences)

SEQ ID NO: 1 - RNA, 19 nt, synthetic construct
  source 1..19: mol_type = other RNA
  modified_base 2: mod_base = OTHER; note = uracil
  modified_base 7: mod_base = OTHER; note = uracil
  modified_base 18: mod_base = OTHER; note = uracil
gtgcaatgagggaccagta

SEQ ID NO: 2 - protein, 21 aa, synthetic construct
CGTHKLTFRASHAVQIVYKGC

(19) United States
(12) Patent Application Publication
(10) Pub. No.: US 2026/0216093 A1
(43) Pub. Date: Jul. 30, 2026

(54) COMPOSITIONS AND METHODS FOR ENHANCED DRUG LOADING INTO LIPID-BASED CARRIERS

(71) Applicant: UNIVERSITY OF NOTRE DAME DU LAC, South Bend, IN (US)

(72) Inventors: Yichun Wang, South Bend, IN (US); Youwen Zhang, South Bend, IN (US); Hsueh-Chia Chang, South Bend, IN (US); Ceming Wang, South Bend, IN (US)

(21) Appl. No.: 19/146,809

(22) PCT Filed: Feb. 26, 2024
(86) PCT No.: PCT/US2024/017222

§ 371 Date: Jul. 9, 2025

Related U.S. Application Data
(60) Provisional application No. 63/480,530, filed on Jan. 19, 2023.

(51) Int. Cl. A61K 9/51; A61K 31/713; A61K 47/69; C12N 15/113

(52) U.S. Cl. CPC A61K 9/5192; A61K 9/5123; A61K 31/713; A61K 47/6923; C12N 15/113; C12N 2310/14

(57) ABSTRACT

Described herein are compositions and methods for enhanced drug loading into lipid-based carriers. The compositions and methods may comprise a drug-bound chiral graphene quantum dot comprising a graphene quantum dot functionalized with a non-aromatic chiral ligand having a single chiral center, wherein a chirality of the chiral graphene quantum dot matches a chirality of a lipid content of the lipid-based carrier. The drug-bound chiral graphene quantum dot permeates into the lipid-based carrier via matching of the chirality of the chiral graphene quantum dot to the chirality of the lipid content of the lipid-based carrier to load the drug into the lipid-based carrier. The compositions and methods achieve a drug loading efficiency of greater than 60% into lipid-based carriers.

### CROSS-REFERENCE TO RELATED APPLICATIONS

This application claims priority to U.S. Provisional Patent Application No. 63/480,530 filed on Jan. 19, 2023, which is incorporated by reference herein in its entirety.

### FEDERALLY SPONSORED RESEARCH

This invention was made with government support under grant number 1UG3-CA241684-01 awarded by the National Institutes of Health. The government has certain rights in the invention.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “092012-0014-WO01_sequence_listing_xml_16 Feb. 2024.xml,” was created on Feb. 16, 2024, contains 2 sequences, has a file size of 4.39 Kbytes, and is incorporated by reference in its entirety into the specification.

### BACKGROUND

Extracellular vesicles (EVs) are a diverse group of vesicles that are enclosed by a membrane and are actively secreted by nearly all types of cells. They are present in various human body fluids, such as blood, urine, saliva, and ascites. Small extracellular vesicles (sEVs), which refer to EVs having a diameter of less than about 150-200 nm, are among the most extensively studied types of EVs due to their crucial roles in numerous physiological and pathological processes. These sEVs are known to transport bioactive components, including nucleic acids and proteins, from donor cells to recipient cells, enabling intercellular communication.

Recently, sEVs have been explored as drug delivery vehicles due to their biological and functional characteristics such as low immunogenicity, long circulation time, nontoxicity, optimal biocompatibility, strong tissue penetration, enhanced targeting effect, and ability to cross the blood-brain barrier. However, production and clinical applications of sEV-based drug delivery vehicles remain elusive due to drug loading challenges. sEV drug loading efficiencies using currently available approaches are relatively low. Most of these approaches result in potential lipid damage, protein denaturation, and precipitation of nucleic acid drugs.

Endogenous drug loading involves specific cell cultures or transfected/programmed call cultures that secrete drug-loaded sEVs. This does not require any auxiliary loading apparatus and is hence quite simple. However, it cannot be used for chemical drugs and the yield of secreted sEVs with biologics is generally low (<30%) due to the myriad of intracellular sEV biogenesis and molecular sorting mechanisms.

Active exogenous drug loading methods such as sonication, electroporation, extrusion, surfactant (saponin) permeabilization, liposome fusion, and freeze-thaw cycles aim to incorporate the drugs after sEV harvesting and isolation. However, as these active methods involve disruption of the sEV bilayer, they can also adversely lead to sEV lysis or fusion. Hence, the size distribution, function, zeta potential, and drug capacity of the cargo-loaded sEVs are sensitive to the loading procedures. The most common electroporation and sonication methods can also damage the loaded cargos, leading to lipid degradation, protein denaturation, and nucleic acid precipitation due to acoustophoretic and electrophoretic effects and their related dipolar force fields at the single-molecule level. In fact, most active exogeneous loading methods potentially damage lipids and denature proteins, which can lead to the degradation of intrinsic sEV structure and alteration of biological functions.

Passive exogenous drug loading by incubation of drugs with extracted sEVs is the least disruptive loading technology among the currently available methods. It is also the most scalable to large-volume production relevant to clinical translations and applications. However, by using diffusion across the lipid bilayer as the only loading mechanism, passive incubation can only be applied to soluble drugs with lipophilic moieties. Consequently, small interfering RNA (siRNA) cargo often need to be conjugated with hydrophobic molecules to facilitate transport. However, other than their potential toxic effects, such hydrophobic moieties often prevent luminal drug loading due to the drug either adhering to the sEV or intercalating at its lipid bilayer. Without the conjugation of hydrophobic moieties, passive loading efficiency of soluble drugs is typically lower than 10%.

Thus, what is needed are novel compositions and methods for more efficient drug loading of EVs and other types of lipid-based carriers for effective delivery of various exogenous therapeutics to cells. Such compositions and methods would be useful in a variety of commercial, research, and clinical applications.

### SUMMARY

One embodiment described herein is a composition for enhanced drug loading into a lipid-based carrier, the composition comprising: a chiral graphene quantum dot comprising a graphene quantum dot functionalized with a non-aromatic chiral ligand having a single chiral center, wherein a chirality of the chiral graphene quantum dot matches a chirality of a lipid content of the lipid-based carrier; and a drug bound to the chiral graphene quantum dot. In one aspect, the graphene quantum dot has a size of about 1 nm to about 15 nm. In another aspect, the graphene quantum dot is functionalized with the non-aromatic chiral ligand through covalent or noncovalent conjugation. In another aspect, the graphene quantum dot is functionalized with about 1 to about 10 molecules of the non-aromatic chiral ligand. In another aspect, the non-aromatic chiral ligand is negatively charged or positively charged at physiological conditions. In another aspect, the non-aromatic chiral ligand comprises a D-amino acid or a L-amino acid. In another aspect, the chiral graphene quantum dot has a zeta potential of about −4 mV to about 4 mV. In another aspect, the chiral graphene quantum dot has a two-dimensional nanosheet structure having left- or right-handed twists characterized by a dihedral angle formed from an outer edge of the chiral graphene quantum dot to a center of the chiral graphene quantum dot, the dihedral angle ranging from about 0° to about 45°. In another aspect, the drug is bound to the chiral graphene quantum dot through π-π stacking, van der Waals interactions, hydrophobic interactions, covalent bond interactions, electrostatic interactions, or combinations thereof. In another aspect, the drug comprises a hydrophobic drug, a hydrophilic drug, or an amphiphilic drug. In another aspect, the drug comprises a nucleic acid, a peptide, a polypeptide, an antibody, a small molecule, or a combination thereof. In another aspect, the drug is a nucleic acid selected from the group consisting of a siRNA, a shRNA, and an antisense oligonucleotide. In another aspect, about 1 to about 30 molecules of the drug are bound to the chiral graphene quantum dot. In another aspect, the lipid content of the lipid-based carrier comprises phosphatidylserines, phosphatidylcholines, phosphatidylinositols, phosphatidylethanolamines, phosphatidylglycerols, sphingomyelins, cholesterols, glycosphingolipids, or combinations thereof.

Another embodiment described herein is a method for enhanced drug loading into a lipid-based carrier, the method comprising: binding a drug to a chiral graphene quantum dot comprising a graphene quantum dot functionalized with a non-aromatic chiral ligand having a single chiral center to generate a drug-bound chiral graphene quantum dot, wherein a chirality of the chiral graphene quantum dot matches a chirality of a lipid content of the lipid-based carrier; and combining the drug-bound chiral graphene quantum dot with the lipid-based carrier to load the drug into the lipid-based carrier. In one aspect, the lipid-based carrier comprises an extracellular vesicle (EV), a small extracellular vesicle (sEV), an exosome, an ectosome, a microvesicle, a liposome, a lipoprotein, a lipid nanoparticle, an exomere, a supermere, or combinations thereof. In another aspect, the lipid-based carrier has a size of about 10 nm to about 500 nm. In another aspect, the lipid content of the lipid-based carrier comprises phosphatidylserines, phosphatidylcholines, phosphatidylinositols, phosphatidylethanolamines, phosphatidylglycerols, sphingomyelins, cholesterols, glycosphingolipids, or combinations thereof. In another aspect, about 1.0×10⁴ to about 1.0×10⁶ molecules of the drug-bound chiral graphene quantum dot are combined with a single molecule of the lipid-based carrier. In another aspect, the drug-bound chiral graphene quantum dot is incubated with the lipid-based carrier for about 1 min to about 30 min at about 20° C. to about 40° C. to load the drug into the lipid-based carrier. In another aspect, the drug-bound chiral graphene quantum dot permeates into the lipid-based carrier via matching of the chirality of the chiral graphene quantum dot to the chirality of the lipid content of the lipid-based carrier to load the drug into the lipid-based carrier. In another aspect, the method achieves a drug loading efficiency of greater than 60% into the lipid-based carrier.

Another embodiment described herein is a drug-loaded lipid-based carrier generated using any of the compositions or methods as described herein.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-I show the principle of chiral graphene quantum dots (GQDs) enhanced drug loading into sEVs. FIG. 1A shows a schematic of the principle. FIG. 1B shows transmission electron microscope (TEM) images of isolated sEVs by an asymmetric nanopore membrane (ANM). FIG. 1C shows characterization of Chiral GQDs by transmission electron microscope (TEM). FIG. 1D shows atomic force microscopy (AFM) and FIG. 1E shows circular dichroism (CD). FIG. 1F shows permeation of chiral GQDs (blue) into sEVs was evaluated by confocal microscope. FIG. 1G shows confocal images of PHK26 labelled sEVs (red) were taken after treated with D-Cys-GQDs (blue). FIG. 1H shows permeation efficiency was quantified by counts of GQDs loaded sEVs (blue) over the total counts of sEVs. FIG. 11 shows size distribution and particle number of GQD-loaded sEVs were measured with nanoparticle tracking analysis (NTA).

FIG. 2A-B show the size effect on permeation of chiral GQDs into sEVs. FIG. 2A shows TEM images and FIG. 2B shows histograms of size distribution of three size ranges of GQDs. FIG. 2C-E show characterization of chiral GQDs under different size ranges. FIG. 2C shows fluorescent spectra excited at 360 nm, FIG. 2D shows CD spectra, and FIG. 2E shows permeation efficiency of size-dependent chiral-GQDs (15 μM) into sEVs (10⁹ particles/mL, L-G: L-Cys-GQDs and D-G: D-Cys-GQDs).

FIG. 3A-E show the effect of ligand on permeability of chiral GQDs into sEVs. FIG. 3A shows photographs of the chiral GQDs depending on various ligands excited by UV light (λₘₐₓ=365 nm). FIG. 3B shows characterization of GQDs and their chiral derivatives in fluorescent spectra excited at 360 nm. FIG. 3C shows CD spectra, and FIG. 3D-E show permeation efficiency of chiral GQDs (7.5 μM) into sEVs (10⁹ particles/mL) imaged by confocal microscopy at room temperature and quantified by analyzing images.

FIG. 4A-C show the effect of chiral GQD concentrations on their permeation into sEVs.

FIG. 4A shows confocal images of sEVs that were incubated with different concentrations of D-Cys-GQDs and then washed with PBS (4° C.) four times under the support of a 100 kDa centrifuge tube. FIG. 4B shows fluorescent spectra of D-Cys-GQDs-loaded sEVs excited at 360 nm. FIG. 4C shows statistical analysis of permeation efficiency of D-Cys-GQDs into sEVs.

FIG. 5A-F show that the chemotherapy drug Doxorubicin (Dox) was loaded into sEVs by D-Cys-GQDs. FIG. 5A shows a schematic illustration of Dox loading into sEVs facilitated by D-Cys-GQDs. FIG. 5B shows a characterization of attachment of Dox (200 μM) directly onto the D-Cys-GQDs (7.5-22.5 μM) by fluorescent spectra (λₘₐₓ=360 nm) and quenching efficiency. FIG. 5C shows a comparison of D-Cys-GQDs and sonication loading strategies by confocal microscopy at room temperature. FIG. 5D shows loading efficiency of Dox into sEVs facilitated by chiral GQDs. FIG. 5E shows confocal images of free Dox and sEVs-Dox (loaded at a ratio of D-Cys-GQDs/Dox of 15 μM/200 μM) uptake by 3T3 cells in vitro (scale bars: 10 μm). FIG. 5F shows cell viability assessed using a CCK-8 assay.

FIG. 6A-H show siRNA sEVs loaded by D-Cys-GQDs for gene therapy. FIG. 6A shows confocal images of siRNA (red) loaded sEVs (green) with facilitation of D-Cys-GQDs. FIG. 6B shows TEM image demonstration of siRNA permeation with the assistance of D-Cys-GQDs. FIG. 6C shows loading efficiency of chiral Cys-GQDs/siRNA complex into sEVs. FIG. 6D shows confocal imaging of DU145 cells treated with 3T3 sEVs for 48 h (scale bars: 20 μm). FIG. 6E-F show Pygo2 mRNA level (FIG. 6E) and protein level (FIG. 6F) in DU145 cells with the treatment of 3T3 sEV loaded with D-Cys-GQDs/Pygo2 siRNA at different density for 48 h. FIG. 6G-H show comparisons of Pygo2 silencing efficiency after treatment with D-Cys-GQDs/Pygo2 siRNA-loaded 3T3, HepG2, or plasma sEVs for 48 h through qPCR (FIG. 6G) and western blot (FIG. 6H).

FIG. 7A-J show characterizations of GQDs and chiral GQDs. FIG. 7A shows TEM images of R-, L-, and D-Cys-GQDs after one week of sample preparation. FIG. 7B shows histograms of the size distribution of L-Cys-GQDs. FIG. 7C shows histograms of the size distribution of D-Cys-GQDs. FIG. 7D shows g-factor of circular dichroism (CD) spectra for L-Cys-GQDs and D-Cys-GQDs. FIG. 7E shows CD spectra of GQDs and R-Cys-GQDs. FIG. 7F shows UV-Vis spectra of GQDs and R-, L-, and D-Cys-GQDs. FIG. 7G shows fluorescent spectra of GQDs and R-, L-, and D-Cys-GQDs. FIG. 7H shows Zeta-potential of pristine GQDs, R-Cys-GQDs, and chiral Cys-GQDs. FIG. 7I shows stability tests of chiral GQDs by CD. FIG. 7J shows TEM images of D-Cys-GQDs after one month of synthesis.

FIG. 8A-C show characterization of sEVs from 3T3 cells. FIG. 8A shows size distribution of sEVs based on analysis of TEM images. FIG. 88 shows particle number and size distribution of 3T3 sEVs samples measured with NTA. FIG. 8C shows western blot analyses of exosomal biomarkers (CD63, CD9, CD81, and HSP70).

FIG. 9A-C show a permeation study of chiral Cys-GQDs into sEVs. FIG. 9A shows fluorescence spectra of sEV before and after 7.5 μM R-GQDs, L-GQDs, and D-GQDs treatments. FIG. 9B shows fluorescence spectra of eluent collected with multiple washing times. FIG. 9C shows the retention rates.

FIG. 10A shows the fluorescent recovery of lysates of sEVs which were loaded by R-, L-, D-Cys-GQDs and stained by PHK26. FIG. 10B shows a TEM image of sEVs loaded with D-Cys-GQDs.

FIG. 11A-B show the size effect of chiral GQDs on permeation into sEVs. FIG. 11A shows CD spectra of L-Cys-GQDs with different sizes. FIG. 11B shows TEM images of damaged sEVs that were treated with D-Cys-GQDs with the largest size (top, 15 μM, 65.7 nm) or with the middle size (bottom, 15 μM, 25.6 nm).

FIG. 12A shows UV-vis absorption of the L/D-Arg-GQDs and L/D-Trp-GQDs. FIG. 12B shows zeta-potential (ζ, mV) of the L/D-Arg-GQDs and L/D-Trp-GQDs.

FIG. 13A-B show TEM images of damaged sEVs that were treated with D-Cys-GQDs at the concentration of either 30 μM (FIG. 13A) or 15 μM (FIG. 13B).

FIG. 14A-C show the effect of D-Cys-GQD concentration on the fluorescence intensity and quenching efficiency of Doxorubicin (Dox). The binding affinity of Dox molecules carried by each D-Cys-GQD was determined by the saturation level of quenching efficiency using fluorescence resonance energy transfer (FRET) assay shown in FIG. 14A. FIG. 14B shows a plot of quenching efficiency of Dox (200 μM) as a function of D-Cys-GQD concentrations. The saturation point of the quenching efficiency was achieved at 15 μM of D-Cys-GQD. Based on the molar concentrations of Dox (200 μM) and D-Cys-GQDs (15 μM) at this saturation point of quenching efficiency, each D-Cys-GQD was able to carry 14 Dox molecules. FIG. 14C shows UV-vis absorbance spectra of Dox, D-Cys-GQDs, and D-Cys-GQDs/Dox complex.

FIG. 15A-E show drug loading via chiral Cys-GQDs/Dox into sEVs. Fluorescence spectra of sEVs are shown after drug loading by R-Cys-GQDs/Dox (FIG. 15A), L-Cys-GQDs/Dox (FIG. 15B), D-Cys-GQDs/Dox (FIG. 15C), or sonication (FIG. 15D) treatments. FIG. 15E shows the retention rates of Dox in sEVs after washing steps.

FIG. 16A-B show confocal images of free Dox, R-, L-, D-Cys-GQDs/Dox and sonication treated sEVs. FIG. 16A shows a confocal image of sEVs loaded by Cys-GQDs/Dox complex (Scale bars: 2 μm). FIG. 16B shows z-stack confocal images of the Dox (red) loaded into sEVs (Scale bars: 5 μm).

FIG. 17A-B show a comparison of Dox loading by D-Cys-GQDs and sonication. FIG. 17A shows TEM images of 3T3 sEVs samples treated with D-Cys-GQDs/Dox or a sonication loading method. FIG. 17B shows particle number and size distribution of 3T3 sEVs samples treated with D-Cys-GQDs/Dox or the sonication loading method measured with NTA.

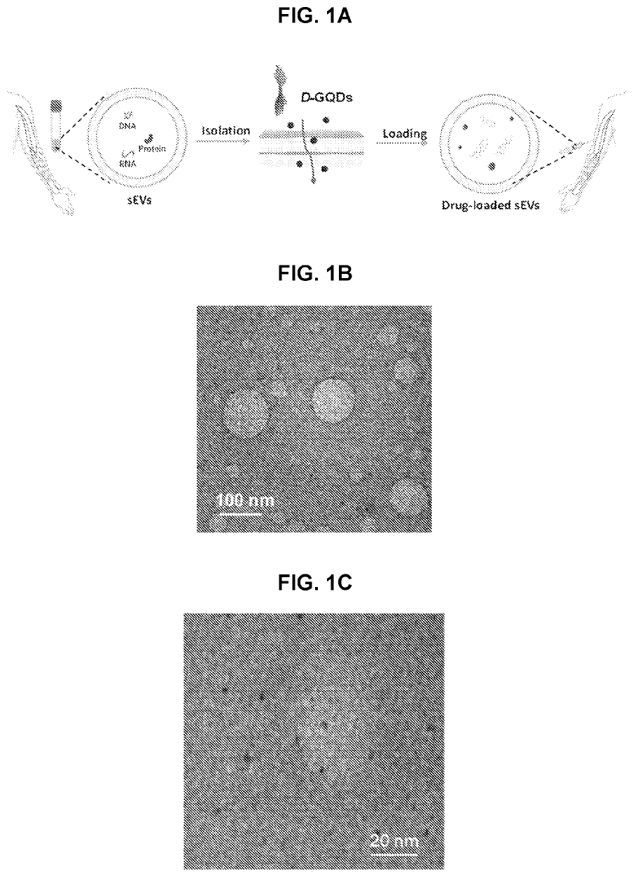

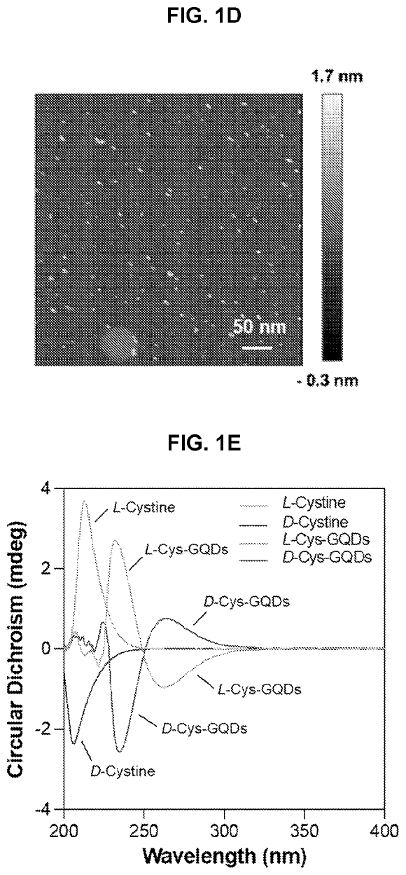

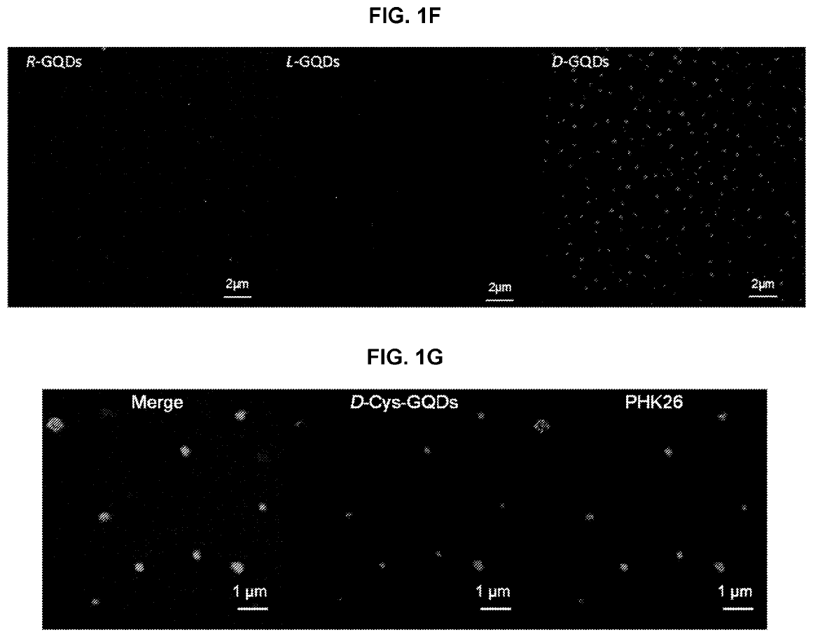

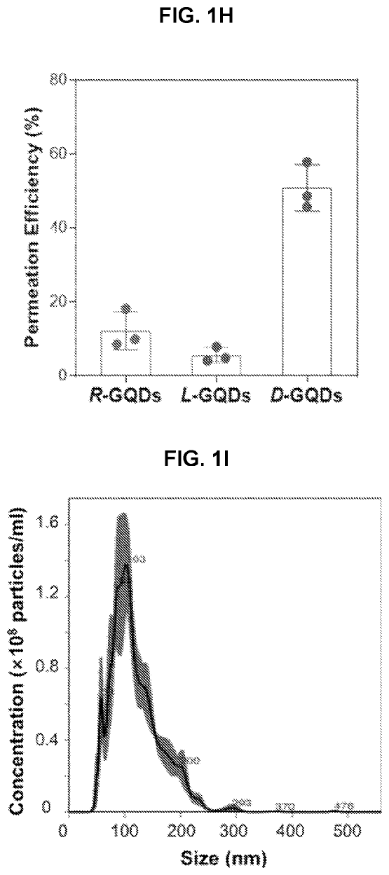

FIG. 1BA-B show the effect of D-Cys-GQD concentration on fluorescence intensity (FIG. 18A) and quenching efficiency (FIG. 18B) of 10 μM siRNA.

FIG. 19A-B show the profile of siRNA loaded sEVs. FIG. 19A shows confocal images of siRNA (red) loaded sEVs with or without facilitation of R-, L-, and D-Cys-GQDs. FIG. 19B shows particle number and size distribution of 3T3 sEVs treated with D-Cys-GQDs/siRNA measured with NTA.

FIG. 20A-D show a comparison of chiral GQD and transfection agent drug loading strategies. FIG. 20A shows the viability of HepG2 cells determined by CCK-8 assay. FIG. 20B shows a confocal image of 3T3-sEVs loaded with siRNA (red) using the transfection agent Upofectamine-2000. FIG. 20C shows a confocal image of 3T3-sEVs loaded with siRNA (red) using D-Cys-GQDs. FIG. 20D shows an analysis of siRNA stability for the two different loading strategies evaluated by a Bioanalyzer system.

FIG. 21A-B show a colorimetric-based assay for the cystine molecule density on chiral GQDs. FIG. 21A shows UV-vis spectra of GQDs/Cysteine complex titrated at different ratios (1:1~1:4). FIG. 21B shows absorbance intensity measured at 375 nm of GQDs/Cysteine complex titrated at different ratios (1:1~1:4).

FIG. 22A-D show characterization of exosomes (sEVs) from three different cell lines: HepG2, 3T3, and HeLa cells. FIG. 22A shows representative TEM images of exosomes from the three different cell lines. FIG. 22B shows size distribution and particle number of exosomes measured by NTA. FIG. 22C shows surface potentials of exosomes analyzed by a Zetasizer. FIG. 22D shows Western blot analysis of exosomal marker expression.

FIG. 23A-D show D-GQDs as representative cargos of exosomes. FIG. 23A shows a schematic illustration of the permeation of D-GQDs into exosomes. FIG. 23B shows confocal laser scanning microscopy (CLSM) images of D-GQDs (blue) loaded into PKH26-labeled exosomes (red) from three different cell lines. FIG. 23C shows permeation of D-GQDs (blue) into the exosomes observed by CLSM. FIG. 23D shows permeation efficiency quantified by counting D-GQD-loaded exosomes over the total number of exosomes. The samples were prepared by combining 12 μM D-GQDs with exosomes (1×10⁹ particles/mL), followed by washing with 1×PBS under the support of 100 kDa centrifugal filter tubes.

FIG. 24A-C show cellular uptake profiles of D-GOD-loaded exosomes derived from three different cell lines into HepG2 cells (FIG. 24A), 3T3 cells (FIG. 24B), and HeLa cells (FIG. 24C) (mean±s.d.). ns: not significant, *: p<0.05, **: p<0.01, ***: p<0.001, ****: p<0.0001. Each channel represents: red for the cell cytosol area and blue for D-GQDs. HepG2, 3T3, and HeLa cell lines were incubated for 6 h with cell culture medium (Control) with a concentration of 0.2×10³ exo/cell. The samples were prepared by loading 12 μM D-GQDs with exosomes (1×10⁹ particles/mL).

FIG. 25A-D show endocytic uptake profiles of exosomes. FIG. 25A shows CLSM images of three cell lines incubated with exosomes derived from different cell lines. Each channel represents: red for the lysosomes and blue for D-GQDs. The quantification of colocalization between D-GQDs and lysosomes was analyzed in HepG2 cells (FIG. 25B), 3T3 cells (FIG. 25C), and HeLa cells (FIG. 25D) (mean±s.e.). ns: not significant, *: p<0.05, **: p<0.01, ***: p<0.001, ***: p<0.0001. HepG2, 3T3, and HeLa cell lines were incubated for 4 h with cell culture medium (Control) with a concentration of 0.2×10³ exo/cell. The samples were prepared by loading 12 μM D-GQDs with exosomes (1×10⁹ particles/mL).

FIG. 26A-E show identification of the surface proteins on exosomes that facilitate receptor-ligand interaction mediated endocytosis. FIG. 26A shows mass spectrometer (MS)-based proteomics. Protein accession numbers were retrieved from the UniProtKB/Swiss-Prot.

Black boxes with [+] corresponded to the proteins that were detected, while the white boxes with [−] indicated undetected. TGF: Transforming growth factor, GalNAc: N-acetylgalactosamine, NRG: Neuregulin, CHC: Clathrin-Heavy Chain, HSP: Heat shock protein. FIG. 26B shows Western blot analysis of TGF-01, GalNAc, and NRG1 expression for three types of exosomes. Relative expression levels of TGF-β1 (FIG. 26C), GalNAc (FIG. 26D), and NRG1 (FIG. 26E) were calculated (mean±s.e.). Data were normalized to the expression level of β-actin. ns: not significant, *: p<0.05, *: p<0.01, ***: p<0.001, ****: p<0.0001.

FIG. 27A-C show membrane fusion between cells and exosomes. CLSM images of HepG2 (FIG. 27A), 3T3 (FIG. 27B), and HeLa (FIG. 27C) cells are shown after 1 h of incubation. Each channel represents: blue for nuclei, green for cellular membrane, red for exosomal membrane, and gray for FRET. Pearson's correlation coefficient (PCC) was quantified for each cell line based on merged images, and FRET evaluation for each cell line is shown by measured fluorescence intensity (ex: 484 nm, em: 565 nm) in cell suspension (mean±s.d.). ns: not significant, *: p<0.05, *: p<0.01, **: p<0.001, “*: p<0.0001. HepG2, 3T3, and HeLa cell lines were incubated with cell culture medium (Control) with a concentration of 0.4×10³ exo/cell.

FIG. 28 shows D-GQDs release and retention from exosomes after 6 h of incubation with HepG2 cells, 3T3 cells, and HeLa cells. Each channel represents: green for the cellular membrane, red for the exosomal membrane, and blue for D-GQDs. The arrows indicate D-GQDs, representing the exosomal cargo released from the exosomes. HepG2, 3T3, and HeLa cell lines were incubated with D-GOD-loaded exosomes at a concentration of 0.2×10³ exo/cell, while the control group was treated with cell culture medium alone. The samples were prepared by loading 12 μM D-GQDs with exosomes (1×10⁹ particles/mL).

FIG. 29A-B show schematic illustrations for the cellular uptake mechanisms of exosomes, depending on their cell-of-origin, followed by cargo release based on the specific cellular uptake mechanism. FIG. 29A shows intraspecies endocytic uptake: exosomes from the same cell-of-origin demonstrated a greater tendency to undergo cellular uptake by parental recipient cells through endocytosis mediated by receptor-ligand interactions, resulting in the entrapment of cargo within lysosomes. FIG. 29B shows cross-species direct fusion uptake: exosomes derived from different cells-of-origin were taken up less by non-parental recipient cells, but primarily through direct membrane fusion, resulting in the direct release of cargo into the cytosol.

FIG. 30 shows a schematic of loading tau-specific N-amino peptide (NAP)-D-GQDs into exosomes for the treatment of tauopathies.

FIG. 31 shows molecular structure images of a tau-specific NAP called Qal (top) and the D-cys-GQDs (bottom).

FIG. 32A-B show the effect of D-GQD concentrations on fluorescence intensity excited at 265 nm (FIG. 32A) and quenching efficiency of the Qal NAP (FIG. 32B).

FIG. 33A-B show fluorescence spectra of exosomes after treatment with 7.5 μM D-GQDs and washing with PBS multiple times (FIG. 33A), and eluents collected after multiple PBS washes (FIG. 33B).

FIG. 34 shows a CLSM image of the permeation of NAP-bound D-GQDs (blue, 15 μM) into exosomes (1×10⁹ particles/mL).

FIG. 35 shows a schematic of a tau biosensor propagation assay for adding inhibitors after fiber seeding.

FIG. 36 shows graphs of the number of intracellular fluorescent puncta relative to control infection wells without inhibitors (Tau infection, %) for each sample. The inhibitors (e.g., Qal-D-GQD-Exo) were added to cells 1 h after tau fibrils.

FIG. 37 shows graphs of the number of intracellular fluorescent puncta relative to control infection wells without inhibitors (Tau infection, %) for bare Qal treatment at concentrations of 1.5 μM to 9 μM.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of the embodiments or elements presented herein, whether explicitly set forth or not. As used herein, “comprising,” is an “open-ended” term that does not exclude additional, unrecited elements or method steps. As used herein, “consisting essentially of limits the scope of a claim to the specified materials or steps and those that do not materially affect the basic and novel characteristics of the claimed invention. As used herein, “consisting of excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest. As described herein, drug-loaded lipid-based carriers can be used to treat or prevent any number of diseases or conditions in a subject in need thereof using one or more drugs or therapeutic agents.

As used herein, the term “loaded” in reference to a “drug-loaded lipid-based carrier” refers to a lipid-based carrier having one or more drugs or therapeutic agents that are fully encapsulated inside the lipid-based carrier (e.g., drug is fully encapsulated in the lumen of the lipid-based carrier); are associated with or partially embedded within an interior lipid content or a lipid membrane of the lipid-based carrier (i.e., partly protruding inside the interior of the lipid-based carrier); or are entirely disposed within an interior lipid content or a lipid membrane of the lipid-based carrier (i.e., entirely contained within an interior lipid content or a lipid membrane of the lipid-based carrier). Thus, in some embodiments, the loaded drug is fully encapsulated inside the lipid-based carrier; is associated with or partially embedded within an interior lipid content or a lipid membrane of the lipid-based carrier; or is entirely disposed within an interior lipid content or a lipid membrane of the lipid-based carrier. In certain non-limiting embodiments, a drug is fully encapsulated inside the lumen of a lipid-based carrier using chiral graphene quantum dots as described herein.

As used herein, the term “lipid-based carrier” refers to an extracellular molecule having at least one lipid moiety and that is capable of being loaded with and carrying a drug-bound chiral graphene quantum dot as described herein. Lipid-based carriers, as described herein, can be spherical or non-spherical in shape. In some embodiments, a lipid-based carrier as described herein may have a size ranging from about 10 nm to about 500 nm, such as from about 30 nm to about 150 nm. In some embodiments, a lipid-based carrier as described herein may have a size that is greater than about 500 nm, such as from about 500 nm to about 1 μm. In some embodiments, a lipid-based carrier as described herein may comprise an extracellular vesicle (EV), a small extracellular vesicle (sEV), an exosome, an ectosome, a microvesicle, a liposome, a lipoprotein, a lipid nanoparticle, an exomere, a supermere, or combinations thereof. In certain non-limiting embodiments, the lipid-based carrier is an exosome or sEV.

As used herein, the terms “exosome,” “small extracellular vesicle,” and “sEV” are used interchangeably and refer to cell-derived vesicles having a diameter of between about 30-150 nm, such as between about 40 and 140 nm, for example, a diameter of about 50 nm, 60 nm, 70 nm, 80 nm, 90 nm, 100 mm, 110 nm, 120 nm, or 130 nm. Exosomes may be isolated from any suitable biological sample from a mammal, including but not limited to, whole blood, serum, plasma, urine, saliva, breast milk, cerebrospinal fluid, amniotic fluid, ascitic fluid, bone marrow and cultured mammalian cells (e.g., immature dendritic cells (wild-type or immortalized), induced and non-induced pluripotent stem cells, fibroblasts, platelets, immune cells, reticulocytes, tumor cells, mesenchymal stem cells, satellite cells, hematopoietic stem cells, pancreatic stem cells, white and beige pre-adipocytes and the like). As one of skill in the art will appreciate, cultured cell samples will be in the cell-appropriate culture media (using exosome-free serum). Exosomes include specific surface markers not present in other vesicles, including surface markers such as tetraspanins, e.g., CD9, CD37, CD44, CD53, CD63, CD81, CD82 and CD151; targeting or adhesion markers such as integrins, ICAM-1, EpCAM and CD31; membrane fusion markers such as annexins, TSG101, ALIX; and other exosome transmembrane proteins such as Rab5b, HLA-G, HSP70, LAMP2 (lysosome-associated membrane protein) and LIMP (lysosomal integral membrane protein). Exosomes may also be obtained from a non-mammal or from cultured non-mammalian cells. As the molecular machinery involved in exosome biogenesis is believed to be evolutionarily conserved, exosomes from non-mammalian sources include surface markers which are isoforms of mammalian surface markers, such as isoforms of CD9 and CD63, which distinguish them from other cellular vesicles. The term “non-mammal” is meant to encompass, for example, exosomes from microorganisms such as bacteria, flies, worms, plants, fruit/vegetables (e.g., corn, pomegranate), and yeast.

As used herein, a “non-aromatic chiral ligand having a single chiral center” refers to a ligand molecule for conjugation onto a graphene quantum dot as described herein, where the ligand molecule has no aromatic groups (e.g., phenyl groups; benzene rings) and no more than one chiral carbon atom that is bonded to four distinct chemical substituents. The term “aromatic” or “aromatic group,” as used herein, means a monovalent group having a monocyclic ring structure or fused bicyclic ring structure. Monocyclic aromatic groups contain 5 to 10 carbon atoms, such as 5 to 7 carbon atoms, or 5 to 6 carbon atoms in the ring. Bicyclic aromatic groups contain 8 to 12 carbon atoms, such as 9 or 10 carbon atoms in the ring.

As nanoscale EVs secreted by cells, exosomes (sEVs) have a large potential as safe and effective vehicles to deliver drugs into lesion locations. Described herein is an exogenous drug-agnostic chiral graphene quantum dot (GQD) platform for drug loading into lipid-based carriers, including sEVs, based on chirality matching with the membranous and/or interior lipid content of the lipid-based carrier. Hydrophobic, hydrophilic, and amphiphilic chemical and biological drugs can be functionalized or adsorbed onto the described GQDs by π-π stacking and van der Waals interactions, for example. By modulating the specific ligands and the GOD size to optimize the chirality of the GQDs, drug loading efficiencies into lipid-based carriers of greater than 60% can be achieved, which is significantly higher than other reported drug loading techniques.

One embodiment described herein is a composition for enhanced drug loading into a lipid-based carrier, the composition comprising: a chiral graphene quantum dot comprising a graphene quantum dot functionalized with a non-aromatic chiral ligand having a single chiral center, wherein a chirality of the chiral graphene quantum dot matches a chirality of a lipid content of the lipid-based carrier; and a drug bound to the chiral graphene quantum dot.

In one aspect, the graphene quantum dot has a size of about 1 nm to about 15 nm.

In another aspect, the graphene quantum dot is functionalized with the non-aromatic chiral ligand through covalent or noncovalent conjugation.

In another aspect, the graphene quantum dot is functionalized with about 1 to about 10 molecules of the non-aromatic chiral ligand. In some embodiments, a graphene quantum dot may be functionalized with greater than 10 molecules of a non-aromatic chiral ligand, such as up to about 15 or up to about 20 molecules of the non-aromatic chiral ligand per graphene quantum dot. The capacity of chiral ligands functionalized onto graphene quantum dots is greater for graphene quantum dots having smaller sizes (e.g., 1-3 nm).

In another aspect, the non-aromatic chiral ligand is negatively charged or positively charged at physiological conditions.

In another aspect, the non-aromatic chiral ligand comprises a D-amino acid or a L-amino acid. In certain non-limiting exemplary embodiments, the non-aromatic chiral ligand may comprise one or more of D-cysteine, L-cysteine, D-cystine, L-cystine, D-arginine, or L-arginine.

In another aspect, the chiral graphene quantum dot has a zeta potential of about −4 mV to about 4 mV.

In another aspect, the chiral graphene quantum dot has a two-dimensional nanosheet structure having left- or right-handed twists characterized by a dihedral angle formed from an outer edge of the chiral graphene quantum dot to a center of the chiral graphene quantum dot, the dihedral angle ranging from about 0° to about 45°. The described chiral graphene quantum dots have chiroptical activity defined by signature peaks at about 200-400 nm depending on the absorbance of the graphene quantum dot component of the chiral graphene quantum dot in circular dichroism (CD) spectra. These chiroptical bands and g-factor of chiral graphene quantum dots depend on the level of right- or left-handed twists of the two-dimensional nanosheets further confirmed by opposite signs of the cloud peaks adjacent to the feature peaks. The twists of the two-dimensional nanosheet structure of graphene quantum dots can be characterized by the defined dihedral angle from an edge to the center of the graphene quantum dots. This angle ranging from about 0° to about 45⁰ is directly associated with the chirality of the graphene quantum dots (CD intensity) and is thus correlated with the drug-loading efficiency of chiral graphene quantum dots.

In another aspect, the drug is bound to the chiral graphene quantum dot through π-π stacking, van der Waals interactions, hydrophobic interactions, covalent bond interactions, electrostatic interactions, or combinations thereof.

In another aspect, the drug comprises a hydrophobic drug, a hydrophilic drug, or an amphiphilic drug.

In another aspect, the drug comprises a nucleic acid, a peptide, a polypeptide, an antibody, a small molecule, or a combination thereof. In some embodiments, the drug may comprise a single therapeutic agent. In some embodiments, the drug may comprise two (or more) different therapeutic agents.

In another aspect, the drug is a nucleic acid selected from the group consisting of a siRNA, a shRNA, and an antisense oligonucleotide.

In some embodiments, the drug is a biologic therapeutic agent. In some embodiments, the biologic therapeutic agent is selected from an allergen, adjuvant, antigen, or immunogen. In some embodiments, the biologic therapeutic agent is selected from an antibody, hormone, factor, cofactor, enzyme, cytokine, chemokine, vaccine, or toxin. In some embodiments, the biologic therapeutic agent is selected from an oligonucleotide therapeutic agent, such as a single-stranded or double-stranded oligonucleotide therapeutic agent. In some embodiments, the oligonucleotide therapeutic agent is selected from a single-stranded or double-stranded DNA, siRNA, mRNA, antisense RNA, miRNA, LNA, morpholine oligonucleotide, or analog or conjugate thereof. In some embodiments, the biologic therapeutic agent is selected from a diagnostic or imaging biologic agent. In some embodiments, the biologic therapeutic agent is a peptide or peptide conjugate molecule.

In another aspect, about 1 to about 30 molecules of the drug are bound to the chiral graphene quantum dot. The specific number of drug molecules able to bind onto a single chiral graphene quantum dot is dependent on the size, shape, and chemical properties of the drug, as well as the size and type of chiral ligand and size of the resulting chiral graphene quantum dot.

In another aspect, the lipid content of the lipid-based carrier comprises phosphatidylserines, phosphatidylcholines, phosphatidylinositols, phosphatidylethanolamines, phosphatidylglycerols, sphingomyelins, cholesterols, glycosphingolipids, or combinations thereof. The lipid content of the lipid-based carrier may comprise interior lipids, lipid membranes, or a combination thereof. The lipid content of the lipid-based carrier comprises at least one lipid molecule containing at least one chiral center for matching with the chirality of the described chiral graphene quantum dots.

Another embodiment described herein is a method for enhanced drug loading into a lipid-based carrier, the method comprising: binding a drug to a chiral graphene quantum dot comprising a graphene quantum dot functionalized with a non-aromatic chiral ligand having a single chiral center to generate a drug-bound chiral graphene quantum dot, wherein a chirality of the chiral graphene quantum dot matches a chirality of a lipid content of the lipid-based carrier; and combining the drug-bound chiral graphene quantum dot with the lipid-based carrier to load the drug into the lipid-based carrier.

In one aspect, the lipid-based carrier comprises an extracellular vesicle (EV), a small extracellular vesicle (sEV), an exosome, an ectosome, a microvesicle, a liposome, a lipoprotein, a lipid nanoparticle, an exomere, a supermere, or combinations thereof.

In another aspect, the lipid-based carrier has a size of about 10 nm to about 500 nm. In certain non-limiting embodiments, the lipid-based carrier has a size of about 30-150 nm, such as between about 40 and 140 nm, for example, a diameter of about 50 nm, 60 nm, 70 nm, 80 nm, 90 nm, 100 mm, 110 nm, 120 nm, or 130 nm.

In another aspect, the lipid content of the lipid-based carrier comprises phosphatidylserines, phosphatidylcholines, phosphatidylinositols, phosphatidylethanolamines, phosphatidylglycerols, sphingomyelins, cholesterols, glycosphingolipids, or combinations thereof.

In another aspect, about 1.0×10⁴ to about 1.0×10⁶ molecules of the drug-bound chiral graphene quantum dot are combined with a single molecule of the lipid-based carrier. In some embodiments, less than about 1.0×10⁴ molecules of the drug-bound chiral graphene quantum dot are combined with a single molecule of the lipid-based carrier. For example, about 1.0×10² to about 1.0×10³ or about 1.0×10⁴ molecules of the drug-bound chiral graphene quantum dot can be combined with a single molecule of the lipid-based carrier. In some embodiments, greater than about 1.0×10⁶ molecules of the drug-bound chiral graphene quantum dot are combined with a single molecule of the lipid-based carrier. For example, about 1.0×10⁶ to about 1.0×10⁷ or about 1.0×10⁸ molecules of the drug-bound chiral graphene quantum dot can be combined with a single molecule of the lipid-based carrier.

In another aspect, the drug-bound chiral graphene quantum dot is incubated with the lipid-based carrier for about 1 min to about 30 min at about 20° C. to about 40° C. to load the drug into the lipid-based carrier.

In another aspect, the drug-bound chiral graphene quantum dot permeates into the lipid-based carrier via matching of the chirality of the chiral graphene quantum dot to the chirality of the lipid content of the lipid-based carrier to load the drug into the lipid-based carrier.

In another aspect, the method achieves a drug loading efficiency of greater than 60% into the lipid-based carrier. For example, in certain embodiments, the compositions and methods described herein may achieve a drug loading efficiency of greater than 65%, 70%, 75%, or even greater than 80% into a lipid-based carrier.

Another embodiment described herein is a drug-loaded lipid-based carrier generated using any of the compositions or methods as described herein. The drug-loaded lipid-based carriers described herein can be used to treat or prevent any number of diseases or conditions in a subject in need thereof. The drug-loaded lipid-based carriers described herein can also be used in any number of diagnostic or imaging applications.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A composition for enhanced drug loading into a lipid-based carrier, the composition comprising:
a chiral graphene quantum dot comprising a graphene quantum dot functionalized with a non-aromatic chiral ligand having a single chiral center, wherein a chirality of the chiral graphene quantum dot matches a chirality of a lipid content of the lipid-based carrier; and
a drug bound to the chiral graphene quantum dot.

Clause 2. The composition of clause 1, wherein the graphene quantum dot has a size of about 1 nm to about 15 nm.

Clause 3. The composition of clause 1 or 2, wherein the graphene quantum dot is functionalized with the non-aromatic chiral ligand through covalent or noncovalent conjugation.

Clause 4. The composition of any one of clauses 1-3, wherein the graphene quantum dot is functionalized with about 1 to about 10 molecules of the non-aromatic chiral ligand.

Clause 5. The composition of any one of clauses 1-4, wherein the non-aromatic chiral ligand is negatively charged or positively charged at physiological conditions.

Clause 6. The composition of any one of clauses 1-5, wherein the non-aromatic chiral ligand comprises a D-amino acid or a L-amino acid.

Clause 7. The composition of any one of clauses 1-6, wherein the chiral graphene quantum dot has a zeta potential of about −4 mV to about 4 mV.

Clause 8. The composition of any one of clauses 1-7, wherein the chiral graphene quantum dot has a two-dimensional nanosheet structure having left- or right-handed twists characterized by a dihedral angle formed from an outer edge of the chiral graphene quantum dot to a center of the chiral graphene quantum dot, the dihedral angle ranging from about 0° to about 45°.

Clause 9. The composition of any one of clauses 1-8, wherein the drug is bound to the chiral graphene quantum dot through T-u stacking, van der Waals interactions, hydrophobic interactions, covalent bond interactions, electrostatic interactions, or combinations thereof.

Clause 10. The composition of any one of clauses 1-9, wherein the drug comprises a hydrophobic drug, a hydrophilic drug, or an amphiphilic drug.

Clause 11. The composition of any one of clauses 1-10, wherein the drug comprises a nucleic acid, a peptide, a polypeptide, an antibody, a small molecule, or a combination thereof.

Clause 12. The composition of any one of clauses 1-11, wherein the drug is a nucleic acid selected from the group consisting of a siRNA, a shRNA, and an antisense oligonucleotide.

Clause 13. The composition of any one of clauses 1-12, wherein about 1 to about 30 molecules of the drug are bound to the chiral graphene quantum dot.

Clause 14. The composition of any one of clauses 1-13, wherein the lipid content of the lipid-based carrier comprises phosphatidylserines, phosphatidylcholines, phosphatidylinositols, phosphatidylethanolamines, phosphatidylglycerols, sphingomyelins, cholesterols, glycosphingolipids, or combinations thereof.

Clause 15. A method for enhanced drug loading into a lipid-based carrier, the method comprising:
binding a drug to a chiral graphene quantum dot comprising a graphene quantum dot functionalized with a non-aromatic chiral ligand having a single chiral center to generate a drug-bound chiral graphene quantum dot, wherein a chirality of the chiral graphene quantum dot matches a chirality of a lipid content of the lipid-based carrier; and
combining the drug-bound chiral graphene quantum dot with the lipid-based carrier to load the drug into the lipid-based carrier.

Clause 16. The method of clause 15, wherein the lipid-based carrier comprises an extracellular vesicle (EV), a small extracellular vesicle (sEV), an exosome, an ectosome, a microvesicle, a liposome, a lipoprotein, a lipid nanoparticle, an exomere, a supermere, or combinations thereof.

Clause 17. The method of clause 15 or 16, wherein the lipid-based carrier has a size of about 10 nm to about 500 nm.

Clause 18. The method of any one of clauses 15-17, wherein the lipid content of the lipid-based carrier comprises phosphatidylserines, phosphatidylcholines, phosphatidylinositols, phosphatidylethanolamines, phosphatidylglycerols, sphingomyelins, cholesterols, glycosphingolipids, or combinations thereof.

Clause 19. The method of any one of clauses 15-18, wherein about 1.0×10⁴ to about 1.0×10⁶ molecules of the drug-bound chiral graphene quantum dot are combined with a single molecule of the lipid-based carrier.

Clause 20. The method of any one of clauses 15-19, wherein the drug-bound chiral graphene quantum dot is incubated with the lipid-based carrier for about 1 min to about 30 min at about 20° C. to about 40° C. to load the drug into the lipid-based carrier.

Clause 21. The method of any one of clauses 15-20, wherein the drug-bound chiral graphene quantum dot permeates into the lipid-based carrier via matching of the chirality of the chiral graphene quantum dot to the chirality of the lipid content of the lipid-based carrier to load the drug into the lipid-based carrier.

Clause 22. The method of any one of clauses 15-21, wherein the method achieves a drug loading efficiency of greater than 60% into the lipid-based carrier.

Clause 23. A drug-loaded lipid-based carrier generated by the method of any one of clauses 15-22.

### EXAMPLES

### Example 1

Enhanced Drug Loading into sEVs Using Chiral GQDs

### Materials and Methods

Isolation and Characterization of sEVs

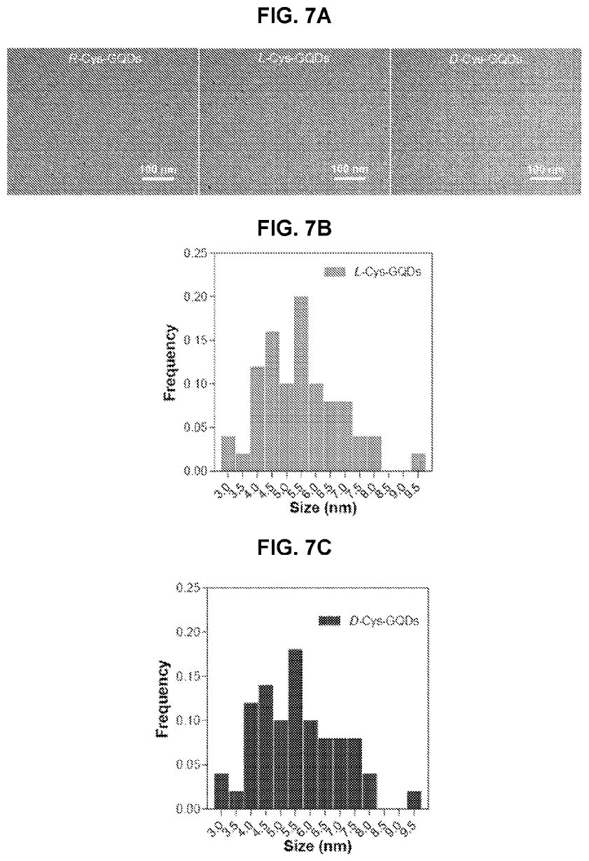

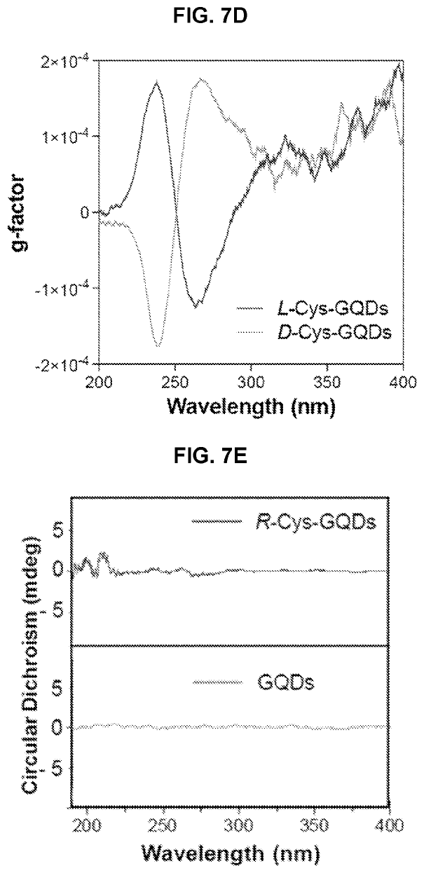

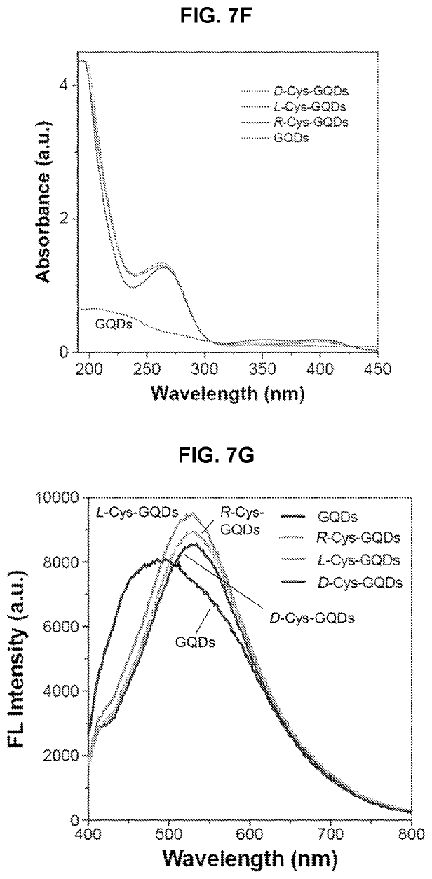

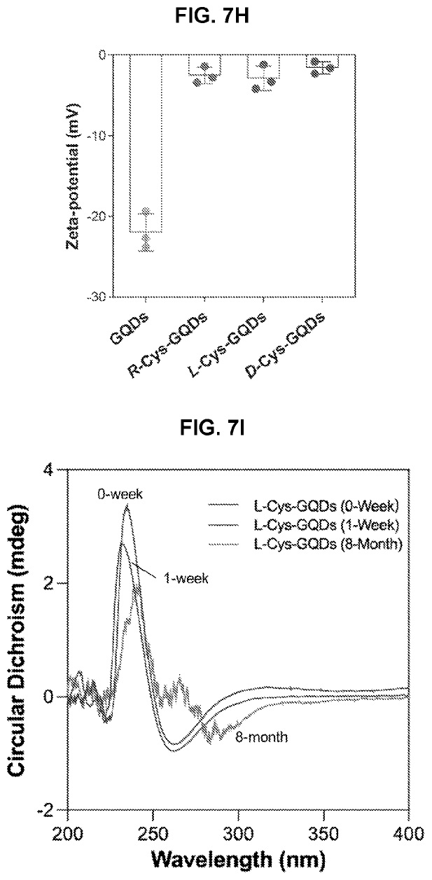

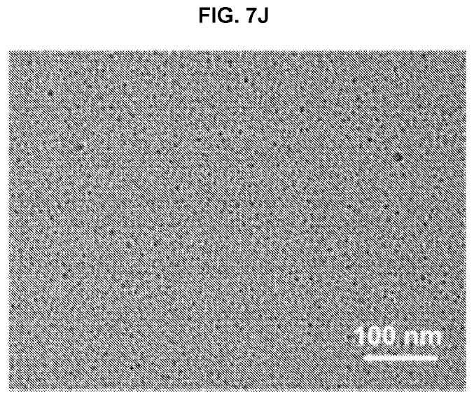

The Hep-G2 and 3T3 cells were purchased from the American Type Culture Collection (ATCC) and propagated in minimum essential medium (MEM, Corning Incorporated, Corning, NY, USA) supplemented with 10% FBS (sEV-depleted) and antibiotics. All cells were maintained in 5% CO₂ at 37° C. and the cell culture medium (CM) was collected for 24 hours. Then, the CM was centrifuged at 500×g for 10 min, 2000×g for 10 min, and 12,000×g for 30 min to remove cells and cell debris. The supernatants were pelleted by ultracentrifugation at 100,000×g for 80 min. However, the yield of ultracentrifugation was found to be too low to produce sufficient sEVs for loading, to the extent that pellets were not found for some cell media samples. Instead, an ultrafiltration method was adopted. The nanopores of ion-track membranes are etched into conic pores to allow high-throughput isolation, enrichment and purification with minimum loss of the sEVs. The data reported are mostly from this Asymmetric Nanopore Membrane (ANM) ultrafiltration technique with a flow rate of 20 mL/hour. A typical NTA characterization of ANM filtered sEV is shown in FIG. 7B-C. sEVs were dissolved with PBS buffer and stored at −80° C. until use.

Exosomal markers CD63 [1:1000; Cell Signaling Technology (CST), USA], and Alix (1:1000; Abcam, UK) and the ER marker calnexin (1:1000; Abcam, UK) were detected by Western blotting. The size distribution and zeta potential of N-Ex were measured by the NanoSight LM10 system (NTA, UK). The PDI of N-Ex was tested by dynamic light scattering (DLS; Malvern Instruments, UK).

Nanoparticle tracking analysis (NTA) measurements were performed with a NanoSight NS300 (NanoSight Ltd., UK) using purified sEVs (100 μL in 1 mL PBS buffer). The mean sEV size distribution (modal hydrodynamic diameter in nm) and sEV concentration (number of EVs enriched from 1 mL of sample in partides/mL) were captured and analyzed with the NTA 3.3 Analytical Software Suite. All procedures were performed at room temperature.

The morphology of sEV was identified by TEM (JEOL 2011) and AFM (Park XE7, Korea). Its structure was further characterized by electron microscopy (EM). Purified sEVs were resuspended in PBS and fixed with 2% paraformaldehyde for 30 min at room temperature. Eight microliters of mixture were then dropped onto EM grids that had been pretreated with UV light to reduce static electricity. After drying for 30 min, sEVs were stained twice (6 min each) with 1% uranyl acetate. The dried grids were examined using an HT7700 (JEOL 2011) transmission electron microscope (TEM) at 120 kV.

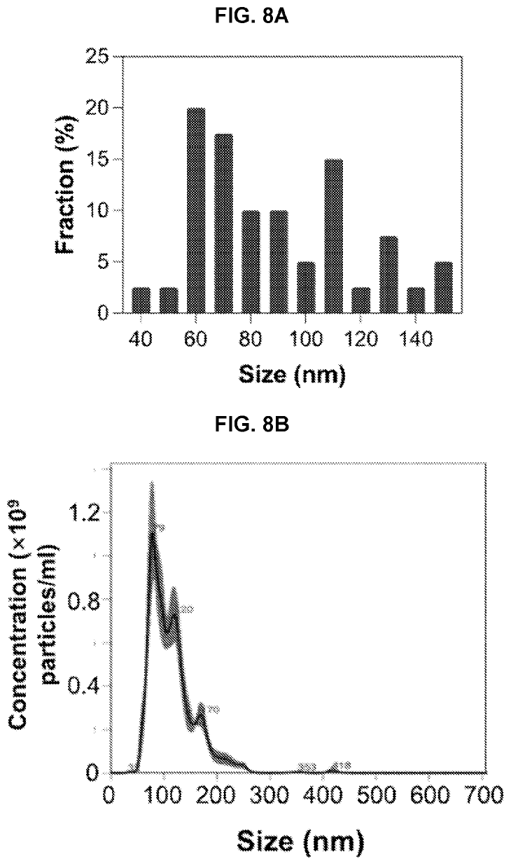

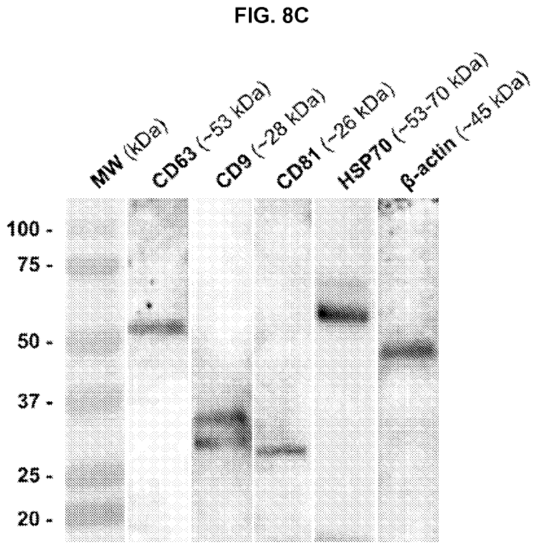

To investigate the permeability of D-GQD into sEVs, the sEVs were harvested from cell cultures of mouse fibroblast cell line (3T3) in vitro. Ultrafiltration and density-gradient ultracentrifugation are often necessary to isolate and purify the sEVs the high shear stress of such isolation procedures often induces protein denaturation and aggregation. For example, low density lipoproteins (LDLs) are known to form aggregates that are in the same 30 to 200 nm size range of sEVs and are difficult to separate from the sEVs. To ensure sEV purity during drug loading by chiral GQD, an ion-track ultrafiltration membrane was employed having pores etched into a conic geometry to reduce shear and fouling by the proteins during their transit. The conic tips of these asymmetric nanopore membrane (ANM) can be etched down to 30 nm, with 3% variation from pore to pore, to prevent sEV transit. Cholesterol assays for a magnetic version of ANM have shown undetectable LDL concentration in the isolated sEVs. Although sEVs from serum-free cell culture media are used in the loading experiments, ANM is nevertheless used to purify and enrich the sEVs. The isolated 3T3 sEV displayed a cup-shaped morphology (FIG. 1F) with an average diameter of 40 to 150 nm (FIG. 8A) based on the statistical analysis of TEM images. Nanoparticle tracking analysis (NTA) showed that the 3T3 sEVs had a narrow size distribution with a mean particle diameter of 116±49 nm (FIG. 8B). These sEVs have shown negative charges with a zeta potential of −13.2±2.7 mV measured by dynamic light scattering (DLS). Further characterization by Western blot confirmed that isolated sEVs had specific exosomal markers CD63 and Alix that were scanned versus s-actin (FIG. 8C). Overall, these results suggest that sEVs were successfully isolated from 3T3 cell lines and exhibited physical and biological features as nanocarriers for drug delivery.

### GQD and Chiral GQD Synthesis

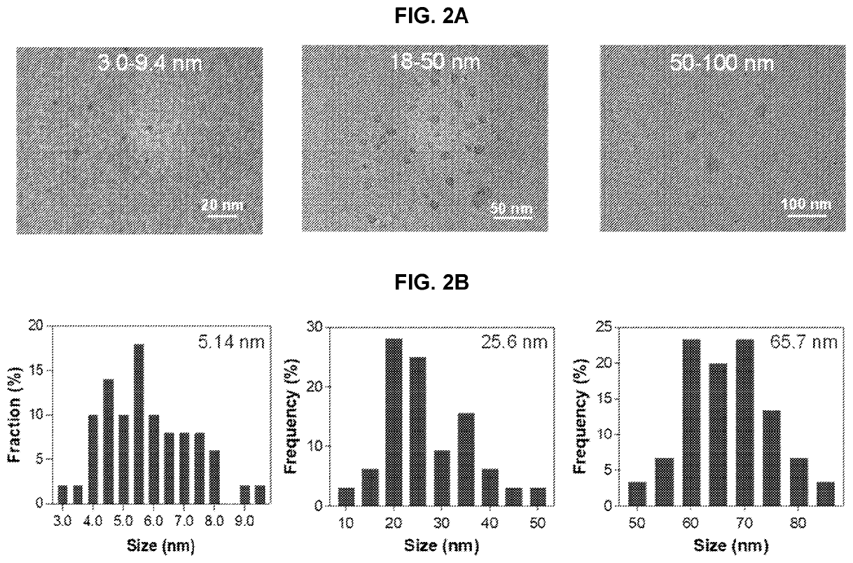

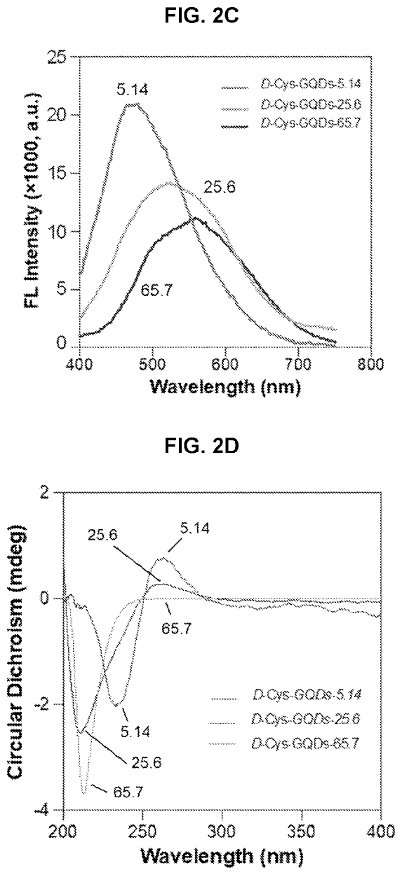

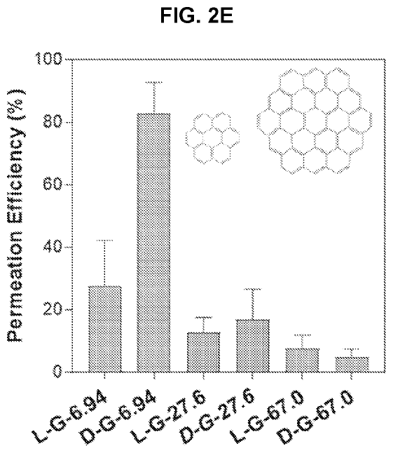

The carbon nanofibers (100 nm), L/D-Cysteine, sulfuric acid, nitric acid, N-(3-Dimethylaminopropyl)-N′-ethylcarbodiimide hydrochloride (EDC, 191.7 g/mol) and N-Hydroxysulfosuccinimide sodium salt (Sulfo-NHS, 217.13 g/mol) were purchased from Sigma-Aldrich. The GQDs were synthesized by a modified protocol from Suzuki et al. CACS Nano 10(2): 1744-1755 (2016) and Zhang et al., Anal. Bioanal. Chem. 410(24): 6177-6185 (2018). Briefly, 0.4 g of carbon nanofibers was dispersed into a 40 mL mixture of sulfuric acid and nitric acid (3:1, v/v) and sonicated for 2 h. The mixture was mechanically stirred for 6 h at room temperature and followed by being heated to 120° C. to continuously react for 10 h. After the reaction, the mixture solution was cooled and diluted with ice DI water and adjusted pH to 8 by adding sodium hydroxide. Then, the GQDs was purified with 3 days dialysis and the final concentration of GQDs was 1 mg/mL. In order to impart chirality to the GQDs, the carboxylic group of GQDs was connected with the amine group of L-(or D-)cysteine by the EDC/NHS method. Briefly, a solution of EDC (20 μL, 100 mM) was added into 2 mL of GOD (100 μM) solution. After 10 min stirring, the 40 μL of Sulfo-NHS (100 mM) was added to the solution, and it was sonicated for 40 min under an ice-water bath. The resulting mixture was treated by a 1 kDa centrifuge tube and rinsed for three times to remove excess EDC and sulfo-NHS. Finally, 40 μL of L- (or D-) cysteine (100 mM) was added into the GQD-NHS ester, and the mixture was stirred for 2 h. The surplus LID-form of cysteine was removed by a dialysis membrane (1 kDa, Fisher Scientific). The density of the cystine molecules on GQDs was determined to be 2.6 using a colorimetric-based assay (FIG. 21) and validating with the absorbance of chiral GQDs at 375 nm (FIG. 7F). Relatively large size GQDs were obtained with average size tunable by reaction time. After 2 h of reaction under the same condition (sulfuric acid and nitric acid were 3:1 in v/v), the size of GQDs were found to be around 3-90 nm (mean value: 50 nm). The solution was separated and cut off by using two sizes of nanoporous membrane (18 and 50 nm). The small size GQDs (<18 nm) in this bench was discard. The middle size (18-50 nm) and large size (50-90 nm) in this bench was collected and then they were modified with L/D-cysteine using the same EDC/NHS method. The size was characterized by TEM (FIG. 2).

The chiroptical activity of the dispersions was measured by CD spectroscopy (J-1700, JASCO), and the chemical reaction progress was monitored by FT-IR spectroscopy (FT/IR-6300, Jasco) and Raman spectroscopy (NRS-5100, Jasco). The absorbance of chiral GQD was analyzed by UV/vis spectroscopy (Agilent, 89090A). The fluorescence property of chiral GQDs was characterized by Infinite M1000 plate reader (Tecan Group). The morphology of chiral GQDs was observed by Transmission Electron Microscopy (TEM) (JEOL 2011). Their surface potential was analyzed by a Zetasizer (Malvern Instruments, Nano ZS).

### Cell Cultures and Viability Assays

3T3 and Hepatocellular carcinoma human cells (HepG2) (ATCC, VA) were maintained with Eagle's minimum essential medium (EMEM) supplemented with 10% fetal bovine serum (FBS) and 1% penicillin-streptomycin (ATCC) in a humidified incubator (MCO-15AC, Sanyo) at 37° C. in which the CO₂ level was maintained at 5% before seeding. All of the medium was filtered using 0.22 μm SteriCup filter assembly (Millipore, USA) and stored at 4° C. for no longer than 2 weeks. For cells incubated with the siRNA-sEVs, and control sEVs, the cells were cultured overnight to allow attachment in a 96-well plate and confocal dishes, washed with FBS-free EMEM, and then incubated with sEV analytes at 37° C. for 1 h in FBS-free medium. After incubation with difference windows, the cells were washed repeatedly with sterilized PBS and maintained in culture medium before further analysis.

Cell viability was assessed using a Cell Counting Kit-8 (CCK-8 assay, Enzo). In brief, 3T3 and HepG2 cells were seeded into a 96-well flat culture plate (Corning). After being cultured overnight, the cells were washed with FBS-free EMEM and incubated with a specific concentration of sEV and exosomal relative in FBS-free medium at 37° C. for 12, 24, 36, or 48 h. The cells were then washed three times with sterilized PBS and incubated with fresh medium containing 10% FBS overnight. The cells were then washed with PBS, and FBS-free EMEM (500 mL) before adding assay reagent. After incubation for 20 min at 37° C., the cell proliferation and cytotoxicity were measured by absorbance at 460 nm. The background was measured by 3T3 and HepG2 cells cultured in FBS-free EMEM only.

### Loading Therapeutic Cargo

To load the sEVs with Dox, 150 μL of purified sEVs and 50 μL of complex (40 μM D-Cys-GQDs: 560 μM Dox) were gently mixed at 4° C. and the mixture was incubated at room temperature for 20 min to ensure the fully permission. To load the sEVs with siRNA, 150 μL of purified sEVs and 50 μL of complex (40 μM D-Cys-GQDs: 80 μM siRNA) were gently mixed at 4° C. and the mixture was incubated at 37° C. for 20 min to ensure the fully permeation. Recovery was assessed by analysis of confocal image as described above. sEVs were then washed with cold PBS four times under the supporting of 100 kDa centrifuge tube to remove unincorporated free D-Cys-GQDs/siRNA. The sEVs loaded with siRNA were quantified for the encapsulated genomic drug by detecting the intrinsic fluorescence of labeled siRNA using Infinite M1000 plate reader (Tecan Group) at 608 nm with excitation at 588 nm and image analysis of confocal microscope under the excitation at 561 nm.

### Determination of the Permeation Efficiency of Chiral GQDs

The permeation efficiency of GQDs in sEVs were indirectly determined by statistically analyzing the count of blue-fluorescent light-up sEVs (caused by permeation) under confocal microscopy over the total concentration of sEV after loading. In brief, 5 μL loaded sEV sample was fully covered with 18 mm×18 mm cover glasses (Corning®, square, No. 1) and scanned under 100× Objective with normal field size 150 μm×150 μm. Four Z-stacks of images were captured through random domains. Each Z-stack contained around 20~30 images from presenting to disappearing fluorescent dots with a step size of 0.125 μm. Then, captured images were analyzed using ImageJ. The total fluorescent sEV particles (TFEPs) were counted by settings with manually adjusted thresholds and matching the size of sEVs. Colocalized fluorescent sEV particles (CFEPs) of Z-stack images was counted based on centers of mass-particles coincidence by using the JACOPx Plugin. The Permeation efficiency into sEVs was calculated using the following formula:

$$Permeation efficiency ( \% ) = \frac{\sum ( TFEPs - CFEPs )}{4 \times TEC} \times \frac{18 mm \times 18 mm}{150 µm \times 150 µm} \times \frac{1}{5 µL} \times 100 ,$$

where TEC is the total sEV concentration (particles/mL) that is measured by NTA.
Determination of Loading Efficiency of Drug in sEVs

While the success of the drug loading procedure was mostly reported in the form of loading efficiency (the percentage of total available drug that has been encapsulated within EVs) or loading capacity (the amount of drug loaded per mass of particles), it is only applicable for reflecting the concentration of active drugs and cannot realistically be applied for defining reproducible protocols of the exogenous loading of sEVs. By taking the encapsulation efficiency of liposome formulation, loading efficiency of sEV, which measures the percentage of active sEVs that successfully encapsulate drugs, was used in the design of sEV-based drug delivery systems. Same as the GQDs permeation, the loading efficiency of Dox and siRNA in sEVs were indirectly determined by statistically analyzing the count of red fluorescent light-up sEVs (caused by loading) under confocal microscopy over the total concentration of sEV after loading. The loading efficiency into sEVs was calculated using the following formula:

$$Loading efficiency ( \% ) = \frac{\sum ( TFEPs - CFEPs )}{4 \times TEC} \times \frac{18 mm \times 18 mm}{150 µm \times 150 µm} \times \frac{1}{5 µL} \times 100 ,$$

where TEC is the total sEV concentration (particles/mL) that is measured by NTA.

### Western Blotting

3T3 cell-derived sEVs were added to 1×RIPA buffer (Cell Signaling) to lysate, and samples were separated by SDS-PAGE. The separated proteins were transferred to a nitrocellulose membrane (Biorad) and treated with anti-CD9 antibody, anti-CD63 antibody, anti-CD81 antibody, anti-CD9 antibody (EXOAB-KIT-1, SBI), and anti-β-actin antibody (Santa Cruz Biotechnology). A secondary anti-rabbit antibody labeled with horseradish peroxidase (SBI) and anti-mouse HPR-linked antibody (Cell Signaling) were used, and immunoreactive species were detected by a Clarity Max Western ECL Substrate (Biorad).

Stability Testing of siRNA

For conventional loading of siRNA into sEVs, siRNA and chemical transfection reagent (Lipofectamine-2000, ThermoFisher) were mixed at a 2:1 ratio followed by incubation for 15 min at room temperature. Then, an sEV suspension was added to the siRNA-Lipofectamine complex and incubated at 37° C. for 20 min. sEVs were then washed with PBS four times with a 100 kDa centrifuge tube to remove the free siRNA-Lipofectamine complex. A bioanalyzer (2100 Bioanalyzer System, Agilent) was used to measure the size of siRNA before and after loading into EVs.

### Example 2

Investigation of Chiral GQD Permeation into sEVs

To investigate the permeation efficiency of chiral GQDs into sEVs (FIG. 1A-B), chiral GQDs were first synthesized that were derived by surface modification with L/D-cysteine (LUD-Cys-GQDs) using a previously reported method. The structures of derived L/D-Cys-GQDs were confirmed by the combination of spectroscopy and microscope. Transmission electron microscope (TEM) images showed that L/D-Cys-GQDs has a size distribution of 3.0-9.4 nm (FIG. 1C and FIG. 7A). Atomic force microscopy (AFM) images (FIG. 1D) confirmed the size range of L/D-Cys-GQDs in TEM. The thickness of L/D-Cys-GQDs was within 2 nm verified by AFM (FIG. 1D), which was corresponding to a single layer of chiral GQDs with enhanced height from helical buckling (twisting) of the pristine GQDs (~1 nm). L- and D-Cys-GQDs showed positive and negative peaks at 236 nm respectively (FIG. 1E) in circular dichroism (CD) spectra. These chiroptical bands and g-factor (FIG. 7B) of chiral GQDs showed opposite signs depending on the reveal of right- and left-handed twists of the 2D nanosheets respectively, matching the chirality of L/D-cysteine used for conjugation to the GQDs. Cloud peaks at 264 nm indicated covalent bonding of cysteines on the edge of GQDs. Whereas the as-synthesized GQDs, and R-Cys-GQDs (i.e., made with racemic mixture of L- and D-cysteine) displayed no chiroptical activity in CD spectra (FIG. 7E) at either a high-energy peak at 220-250 nm or a low-energy peak at 250-300 nm. Furthermore, the UV-Vis spectra (FIG. 7D) of the chiral GQDs revealed two distinct absorption bands centered at 270 nm (ascribed to π-π* transitions in the sp²-hybridized carbon core) and 375 nm (attributed to the π-π* transitions or carboxyl groups). It indicated partial relaxation of exciton confinement compared to pristine GQDs with a peak at 225 nm due to the hybridization of the aromatic system of GQDs with the atomic orbitals on cysteine moieties. Being excited by photons with λex=360 nm, pristine GQDs showed the emission at 480-540 nm, while L-, and D-forms of GQDs displayed strong emission at 500-550 nm (FIG. 7E). The red shift (~26 nm) after the modification with the amino acids in the photoluminescent (PL) peaks was caused by charge transfer between functional groups and the graphene carbon core of GQDs, which narrowed the band gap. The zeta-potential (ζ) of as-synthesized GQDs was −21.8±4.2 mV (FIG. 7F). After attachment of cysteine moieties, ζ-potentials of L- and D-form chiral GQDs became −3.3±2.1 mV and −1.6±1.8 mV (FIG. 7H), respectively. Such reduction of surface charge from GQD to L/D-Cys-GQDs was consistent with amidation of negatively charged carboxyl groups (—COOH) at the edges of GQDs while retaining a substantial degree of ionization. Taken together, the combined results supported the successful synthesis of chiral GQDs.

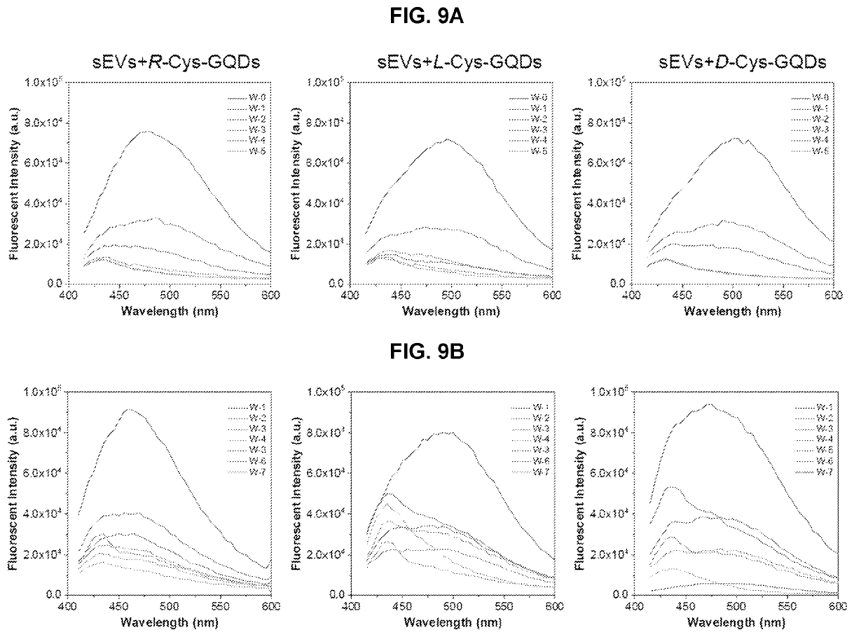

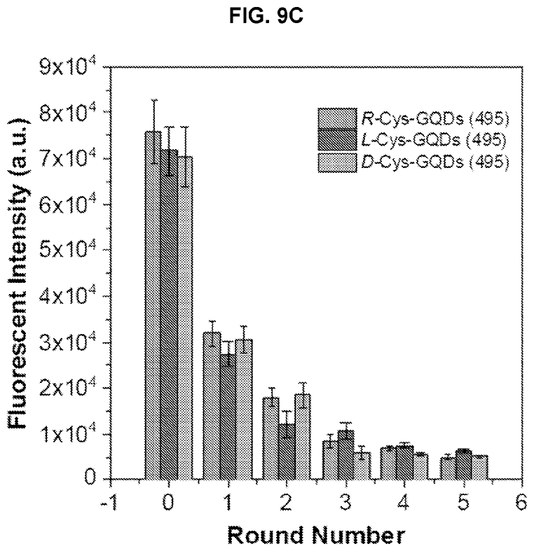

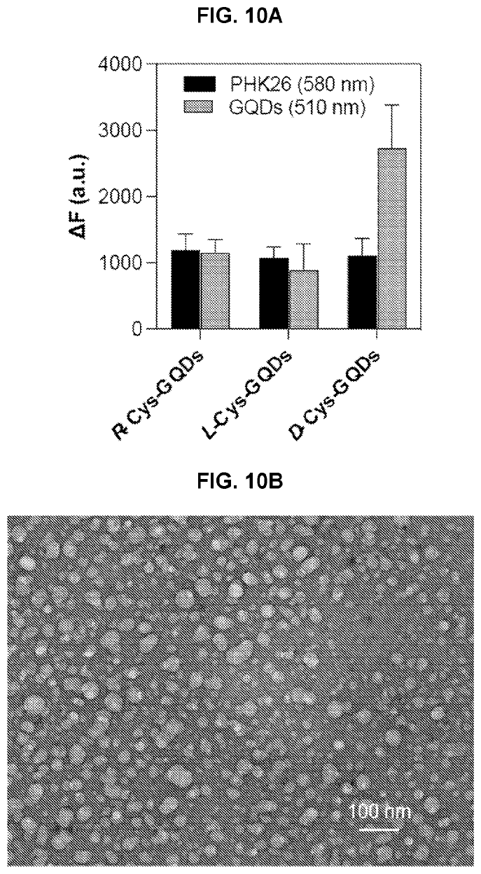

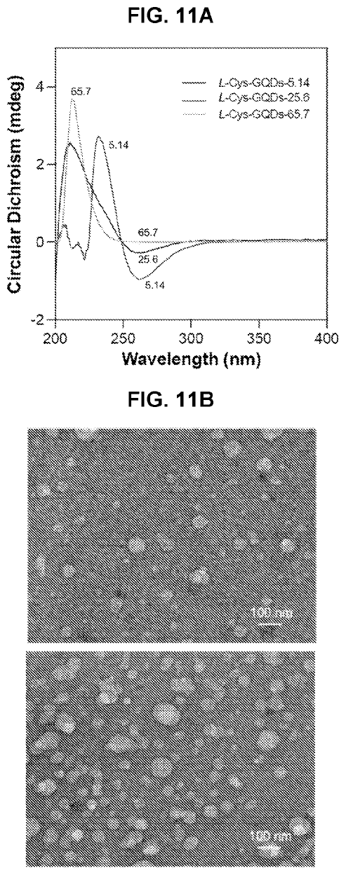

Following synthesis and characterization of L/D-Cys-GQDs, the permeation of chiral Cys-GQDs into sEVs isolated from cell culture media of 3T3 cell lines was investigated (FIG. 1B and FIG. 8). As-synthesized GQDs and their chiral derivates (7.5 μM) were incubated with 3T3 sEVs (1.09×10⁹ particles/mL) in phosphate-buffered saline (PBS) buffer (pH 7.4) at room temperature under a static condition. The permeation of GQDs and their chiral derivatives into sEVs were examined by confocal imaging based on the innate fluorescence of GQDs at 480-540 nm. sEVs after incubation with GQDs were washed with PBS buffer under the hold of 100 kDa centrifuge tube until the fluorescence intensity of GQDs in the mixture decreased to a constant (FIG. 9). The permeation of chiral GQDs into sEVs was detected by the accumulation of innate fluorescence of GQDs (around 525 nm, marked as blue) in the sEVs using confocal microscope. Cys-GQDs with right-handed chirality (D-Cys-GQDs) showed the significantly higher density of blue dots (the accumulation of GQDs) than achiral R-Cys-GQDs and left-handed L-Cys-GQDs (FIG. 1F) under the same concentration of the sEVs (10⁸ particles/mL). To verify that the observed fluorescent signals of GQDs were inside sEVs, sEVs were labelled by staining their membranes using PKH26 that is a red fluorescent lipid membrane dye. The colocalization of PHK26 labeled sEV (red) and D-Cys-GQDs (blue) shown in FIG. 1G demonstrated the permeation and accumulation of D-Cys-GQDs in the sEVs. To note, some point source (100~500 nm) in the fluorescent image was about twice larger than the actual size of the objects (30~200 nm) due to the diffraction effects and resolution limitation of optical microscope (180 nm laterally and 500 nm axially), thus they were within the dimension range of sEVs. In order to quantify the permeation efficiency of GQDs into sEVs, a counting method was developed based on image analysis to reflect the loaded sEVs at single sEV level. In short, the permeation efficiency of GQDs in sEVs were determined by statistically analyzing the total amount of GQDs loaded sEVs (blue dots larger than 30 nm as a threshold) over the total counts of sEVs from nanoparticle tracking analysis (NTA). D-Cys-GQDs exhibited significantly higher permeating efficiency (52.4±8.2%) than R-Cys-GQDs (13.8±4.7%), while L-Cys-GQDs (5.7±2.2%) showed very limited permeation (FIG. 1H). Most importantly, the size and morphology of sEVs retained integrity after the loading procedure by D-Cys-GQDs based on TEM and NTA results (FIG. 10 and FIG. 11). This result aligned with the previous findings by Molecular Dynamics (MD) simulation that D-Cys-GQDs had a stronger tendency to penetrate the cellular lipid membrane of mammalian cells, than L-Cys-GQDs. Such transport phenomenon is potentially attributed to the twist of 2D nanosheet that gives rise to nanoscale chirality with single chiral center, resulting in the interactions between chiral GQDs and lipid membrane of sEVs.

### Example 3

The Effect of Size on the Permeability of Chiral GQDs into sEVs

The interaction of GQDs with lipid bilayer membrane depends on the size of GQDs. In particular, small GQDs (<13.3 nm) are able to enter the lipid bilayers while maintain the membrane intact. However, larger GQDs tend to deform the membrane with the formation of hemisphere vesicles and cause potential damage. Moreover, the chirality originated by lattice distortion at nanoscale are influenced by the size of NP lattices, thus may affect the permeation efficiency of chiral GQDs in sEVs. Here, the effect of L/D-Cys-GQDs with three different sizes was investigated on their permeation into sEVs. The average sizes of the three GQD samples were 5.14 nm, 25.6 nm, and 65.7 nm (FIG. 2A-B) in this study. Due to the same tendency of fluorescence spectra and CD spectra (FIG. 11A) of L/D-Cys-GQDs, D-Cys-GQDs that has higher permeation efficiency was demonstrated in FIG. 2 to show the size effect. With the size increase of chiral GQDs, bathochromic shift was observed in fluorescent emission spectra of D-Cys-GQDs (excited at 360 nm, FIG. 2C). The chirality corresponding to the nanoscale distortion was observed at a low-energy peak (250-300 nm) in CD spectra. Smaller D-Cys-GQDs (5.14 nm) exhibited higher chiroptical activities at 250-300 nm than the larger ones (25.6 nm and 65.7 nm). This was further confirmed by the CD peak at 265 nm of smaller D-Cys-GQDs associated with Cotton effect (FIG. 2D). This phenomenon was potentially due to larger dihedral angles in the twisted molecular structures of the smaller chiral GQDs according to a previously reported study. In addition, there were more terminal —COOH groups on larger GQDs, which increased the probability of anomalous and asymmetric bindings of cysteines, diminishing the symmetry-breaking perturbation of chiral edge ligands to electronic states of graphene nanostructures. Following the characterizations of chiral Cys-GQDs with different sizes, the permeation of these L/D-Cys-GQDs into sEVs isolated from 3T3 cell culture media was investigated by an ultracentrifugation method. The largest chiral GQDs (65.7 nm) showed limited permeation into sEVs according to quantification method of permeation efficiency in the previous session (FIG. 2E). This was potentially because the size of the largest GQDs (65.7 nm) was comparable to the size of sEVs (116 nm, FIG. 7). Instead of passive transport through the membrane, the large GQDs damaged the lipid membranes of sEVs, confirmed by TEM images of sEVs (debris of sEV observed in FIG. 11B). Compared to the smaller D-Cys-GQDs (5.14 nm) with a permeation efficiency of 82.7% into sEVs, the ones with a median size (25.6 nm), has a low permeation efficiency (16.9%). This is consistent with the size distribution of the median GQDs, of which, 9.4% are below 13.3 nm. The permeation efficiency of D-Cys-GQDs was 1.5- to 3-times higher than that of L-Cys-GQDs for both sizes of 5.14 nm and 25.6 nm, indicating the chirality-selective passive diffusion of GQDs through exosomal membranes. Overall, the results suggested that small D-Cys-GQDs can permeate 3T3 sEV membranes most efficiently, which can be attributed to the relative size of GQD to exosomal membrane thickness and size-associated chirality generated from distortion of NP lattices.

### Example 4

The Effect of Ligand on the Permeability of Chiral GQDs into sEVs

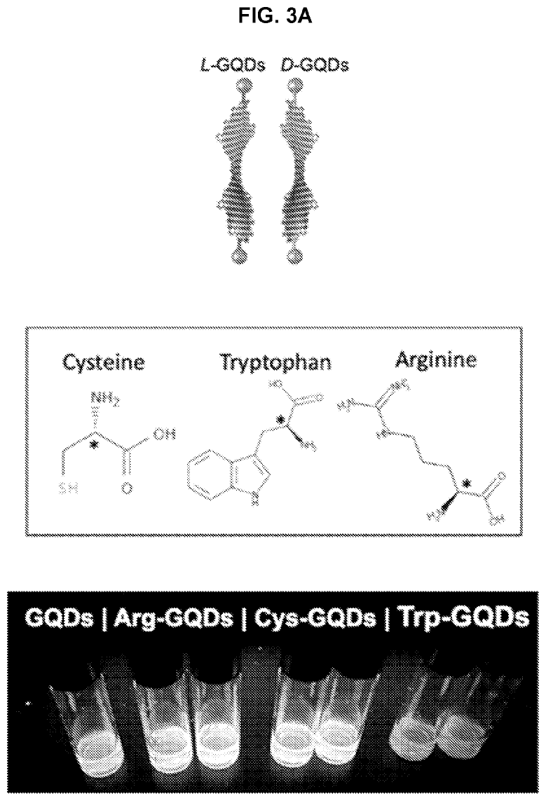

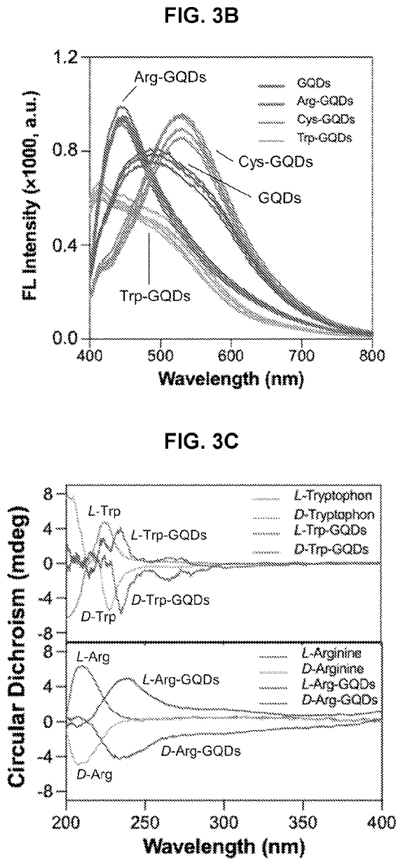

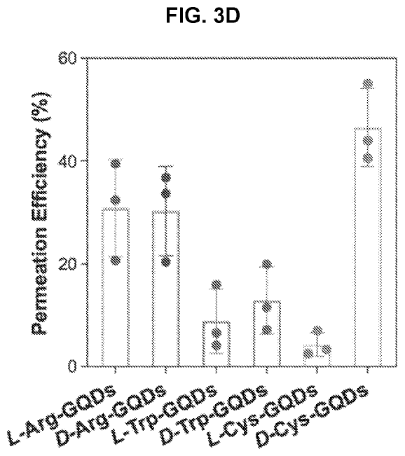

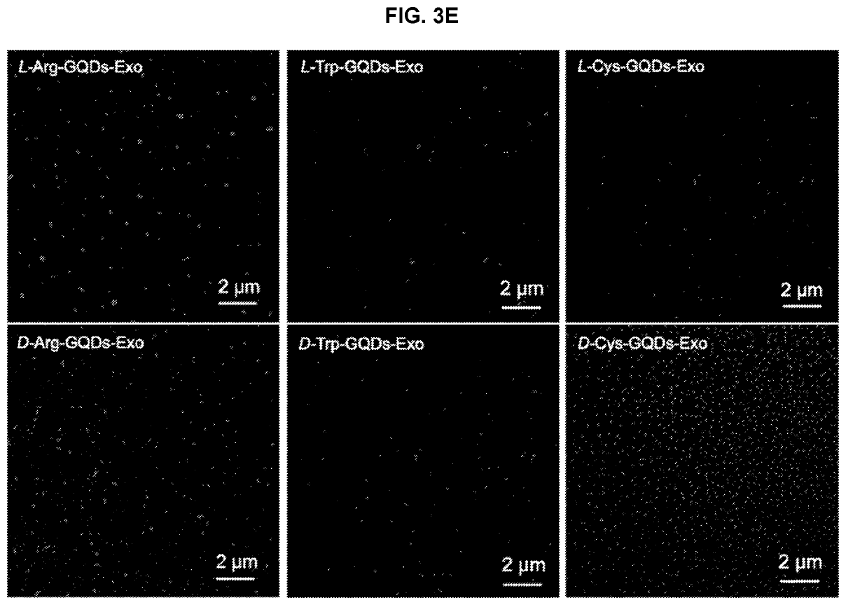

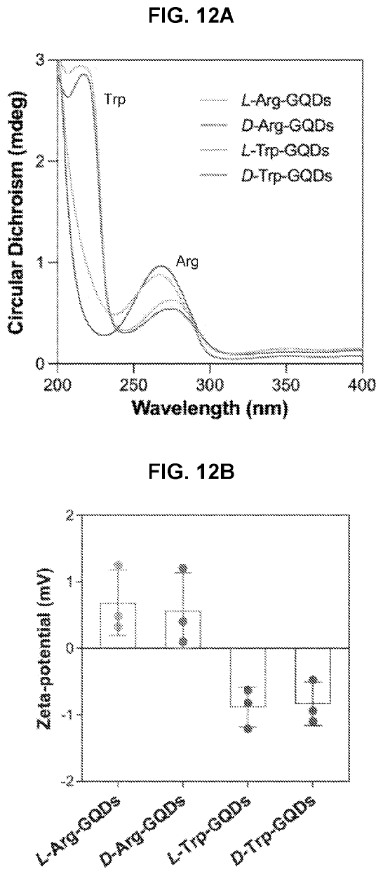

According to previous studies, the chirality of GQDs can be tuned by specific ligand types. This is because chiral ligands having different molecular weights, structures, and charges can change the dihedral angles of physical twists of nanosheets and thus generate different chirality at nanoscale. To investigate the ligand effect on permeation efficiencies of chiral GQDs into sEVs, GQDs were functionalized with two alternative ligands, tryptophan (Trp) and arginine (Arg), primarily because tryptophan is a heavier chiral molecule than cysteine and could result in a larger dihedral angle of nanosheet twist. Moreover, due to the aromatic benzene ring in tryptophan, there are multiple chiral centers generated by this ligand that increases the complexity of chirality for the chiral GQDs. On the other hand, arginine, as a positively charged chiral molecule at physiological pH (~pH 7.0), results in a smaller dihedral angle than cysteine due to the electrostatic interactions with carboxyl groups of GQDs, thus having lower chirality at nanoscale. GQDs were functionalized with arginine or tryptophan by the same coupling reaction for L- and D-Cys-GQDs to generate different chiral GQDs (FIG. 3A). The successful conjugations of arginine and tryptophan onto the edge of GQDs were supported by UV-vis absorption and fluorescent spectra. L/D-Arg-GQDs and L/D-Trp-GQDs both had UV-vis absorption peaks at around 270 nm and 375 nm, similar to Cys-GQDs (FIG. 12A). When excited at 380 nm, L/D-Arg-GQDs displayed strong emission at 400-500 nm (FIG. 3B). A blue shift (~40 nm) was observed compared to as-synthesized GQDs in the PL peaks due to the coupling of electron-donating sidechain that played a role of chromophore on GQDs. L/D-Trp-GQDs showed a broad range of fluorescent emission compared to Cys-GQDs and Arg-GQDs with relatively lower intensity under the same concentration (7.5 μM). This was attributed to the fact that conjugating the large indole group of tryptophan to GQDs weakened the optical property (lower intensity of emission) and the increasing the size of GQDs changed the band gaps. The chirality of these functionalized GQDs was determined and analyzed by CD spectra. CD spectra of both L/D-Arg-GQDs and L/D-Trp-CQDs gave rise to a new symmetrical CD signal at around 240-300 nm. These new peaks were different from those of free Arginine and Tryptophan near 220 nm, indicating the successful synthesis of chiral-GQDs with chiroptical activity (FIG. 3C-D). The zeta-potentials (ζ) of chiral Arg-GQDs were +0.5 mV (L) and +0.3 mV (D), while the zeta-potentials (0 of chiral Trp-GQDs were −1.1 mV (L) and −0.7 mV (D) (FIG. 12B).

The permeation of chiral-GQDs derived from different chiral ligands into sEVs was investigated under the same conditions in the previous session. D-Trp-GQDs reflected slightly higher permeation efficiency (15.63±4.39%) into sEVs than L-Trp-GQDs (9.79±6.48%). However, Trp-GQDs showed a significant reduction of permeation efficiency into sEVs compared with D-Cys-GQDs (FIG. 2D-E). The potential reason is that the tryptophan with a large aromatic molecular structure (FIG. 3A) gave rise to multiple chiral centers and increased the complicity of chiral interactions of chiral GQDs with lipid bilayers of sEVs. Meanwhile, Arg-GQDs presented higher permeation efficiency (L, 31%; D, 29%) (FIG. 3E) than Trp-GQDs, however, there is no selectivity between L- and D-Arg-GQDs. From the CD spectra, the lack of Cotton effect indicated that the chirality from Arg-GQDs originated from the surface enhancement of arginine attached on GQDs noncovalently. This also reflected on the broaden bands between 275 nm and 370 nm without cloud peaks with opposite signs of ligand chirality. This is caused by the partial physical electrostatic absorption of arginine ligand on the GQDs due to the charge of amino acids (+) and as-synthesized GQDs (−). These effects further implicated that nanoscale chirality origin from twist of 2D nanosheet enhance the permeability of biological lipid membrane through selective chiral interactions. Therefore, with the highest permeation efficiency due to the nanoscale chirality, D-Cys-GQDs were chosen as the optimum chiral carriers for drug loading into sEVs in all the subsequent drug loading experiments.

### Example 5

The Effect of Concentration on the Permeability of Chiral GQDs into sEVs

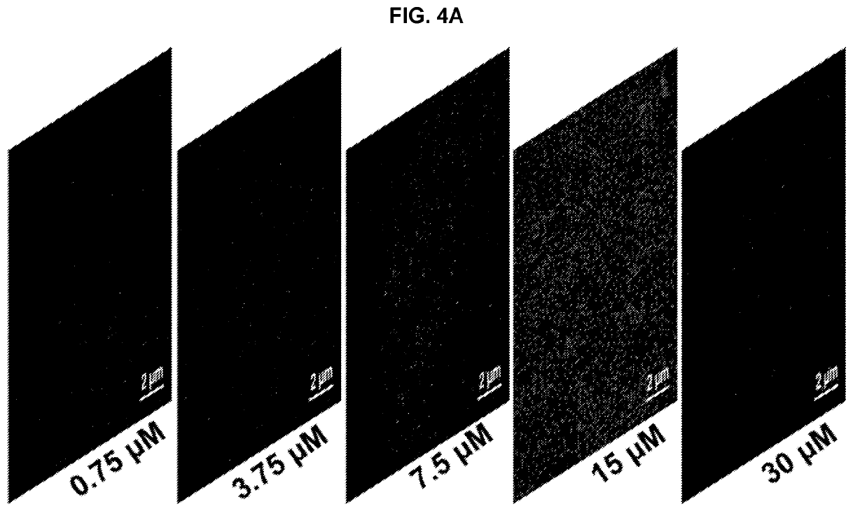

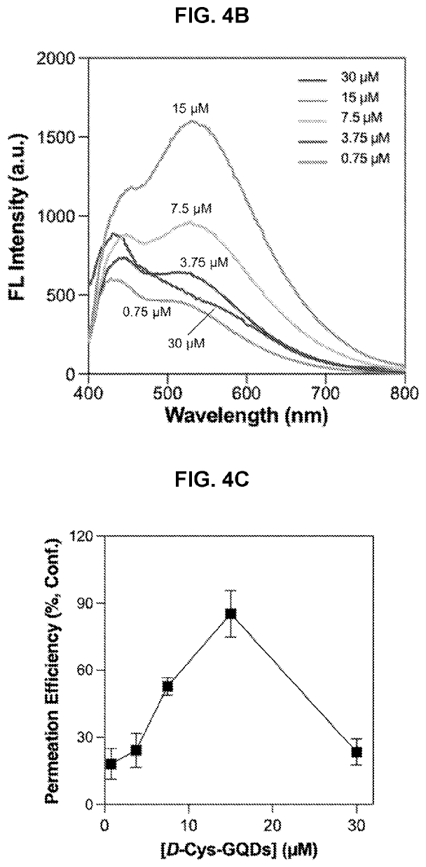

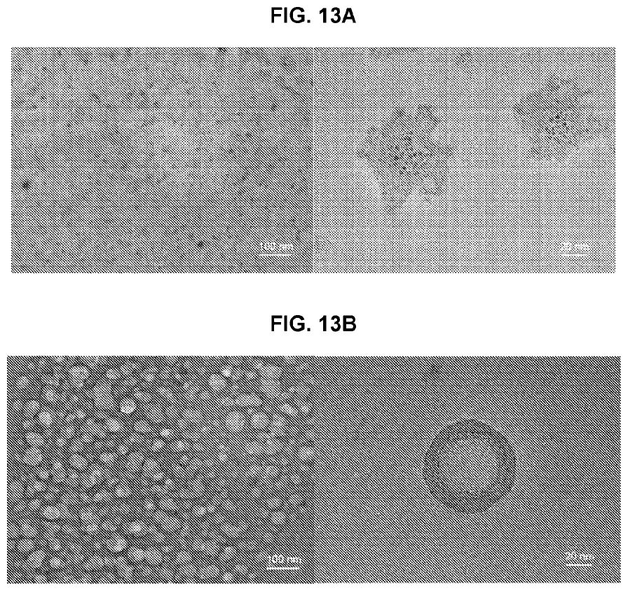

The concentration gradient of nanoparticles (NPs) across the lipid membrane is one of the main driving forces for NP transport through lipid membrane. Therefore, to optimize the permeation efficiency of chiral GQDs as a drug loading vehicle, the permeation efficiency of D-Cys-GQDs at different concentrations in the range of 0.75-30 μM in PBS buffer was investigated. With an increase in the concentration of the D-Cys-GQDs, a larger amount the D-Cys-GQDs entered the sEVs, indicated by more accumulation of D-Cys-GQDs (blue dots, FIG. 4A) and higher retention fluorescent intensity of D-Cys-GQDs-loaded sEV solution (FIG. 4B). Permeation efficiency of D-Cys-GQDs into sEVs enhanced with the increased concentration in the range of 0.75 to 15 μM. However, the permeation efficiency of D-Cys-GQDs dropped significantly when the concentration of D-Cys-GQDs reached up to 30 μM (FIG. 4B). This is potentially attributed to saturation of D-Cys-GQDs within the sEVs that eventually resulted in damage of sEV at an external D-Cys-GQDs saturation concentration of 30 μM (FIG. 13). Overall, the highest permeation efficiency, 85.2±10.3% of D-Cys-GQDs into sEVs was achieved at a concentration of 15 μM (FIG. 4C). All the subsequent drug loading experiments into sEV were conducted at this optimized concentration of D-Cys-GQDs.

### Example 6

D-Cys-GQDs-Enhanced Chemotherapy Drug Loading into sEVs

After optimizing the permeability of chiral GQDs into sEVs, the loading efficiency of a common hydrophobic chemotherapy drug, Doxorubicin (Dox), facilitated by D-Cys-GQDs was evaluated. Dox-loaded sEVs as a promising nanomedicine have shown enhanced therapeutic effects compared to the commercially available Dox-loaded liposomes. However, loading Dox or other common drugs into sEVs remains challenging, which hinder their translational applications as drug delivery carriers.

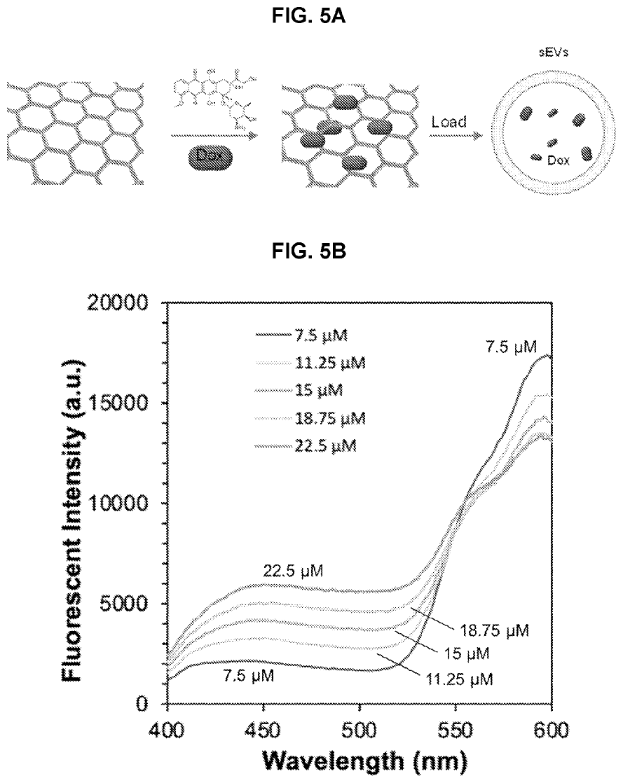

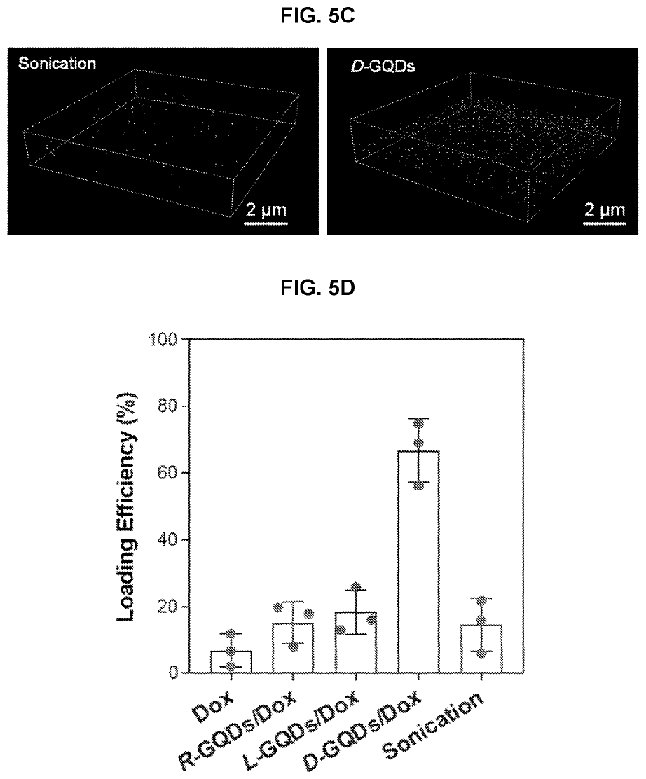

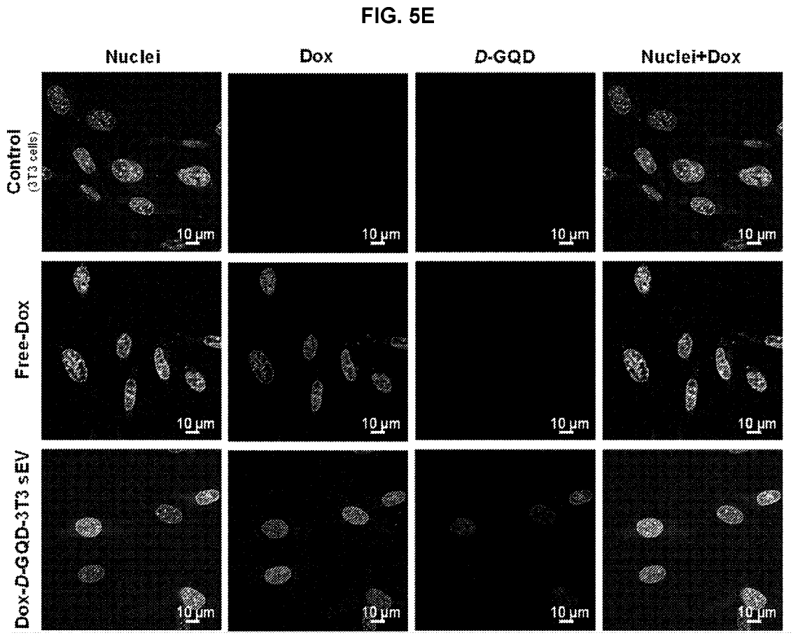

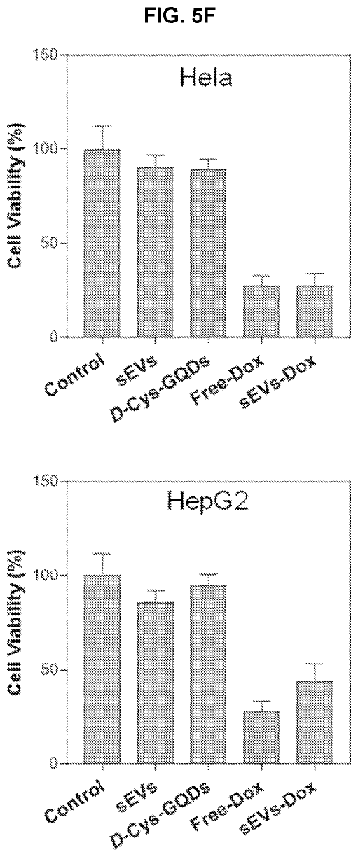

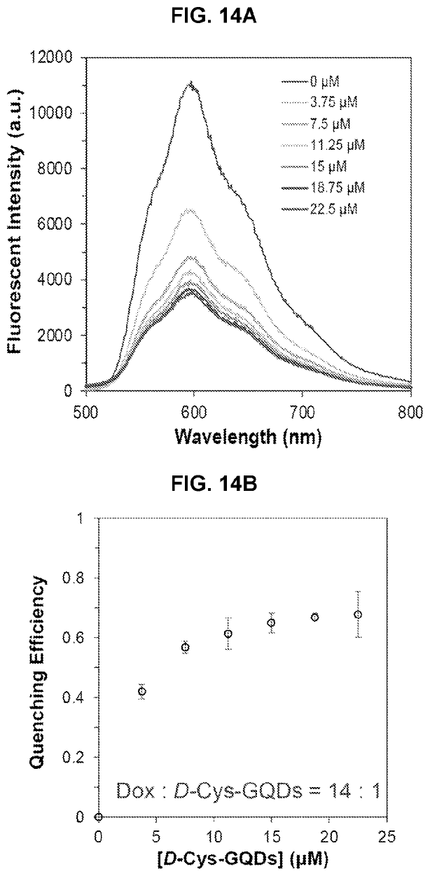

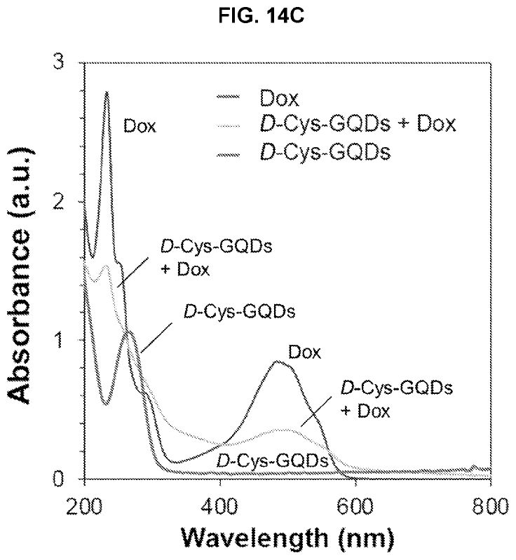

D-Cys-GQDs can carry Dox molecules via π-π stacking between planar surface (carbon ring) of GQDs and anthracene group of Dox molecules. Such D-Cys-GQDs/Dox complex can permeate lipid bilayer of sEV membrane with high efficiency, similar to the permeation efficiency of D-Cys-GQDs into sEVs (FIG. 5A). The amount of Dox molecules carried by each D-Cys-GQD was determined by the quenching efficiency using fluorescence resonance energy transfer (FRET) assay (FIG. 5B). As the concentration of D-Cys-GQDs increased, the quenching efficiency of Dox (200 μM) decreased dramatically. The quenching efficiency of Dox began to reach a plateau when concentrations of D-Cys-GQDs were higher than 15 μM (FIG. 14A), which confirmed the attachment of Dox on the surface of D-Cys-GQDs. Based on the molar concentrations of Dox and D-Cys-GQDs at the plateau of quenching efficiency, each D-Cys-GQD was able to carry 14 Dox molecules (FIG. 14B). D-Cys-GQDs/Dox complex showed absorbance at both featured regions of D-Cys-GQDs and Dox, which further verify the attachment of Dox on the surface of D-Cys-GQDs (FIG. 14C). Taken together, the combined results supported the formation of D-Cys-GQDs/Dox complex.

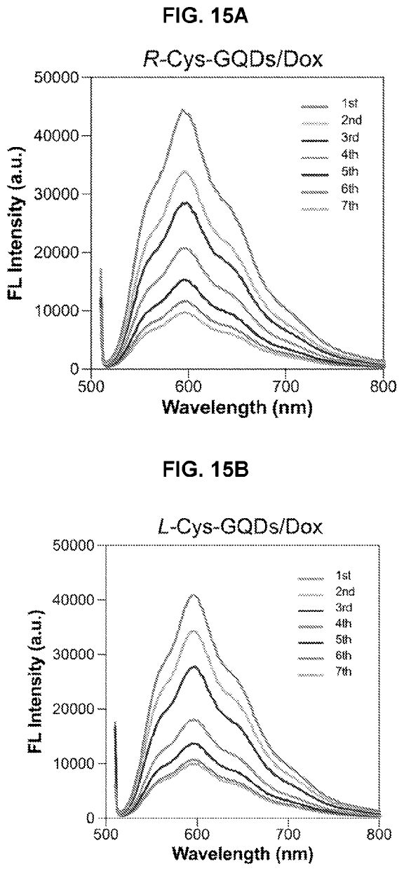

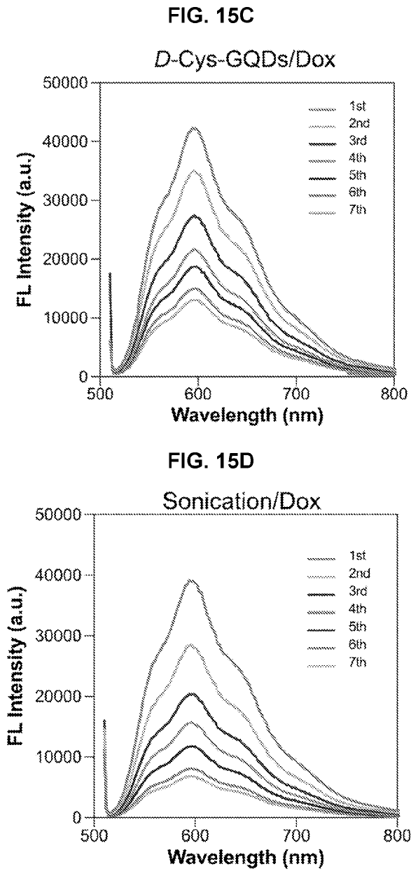

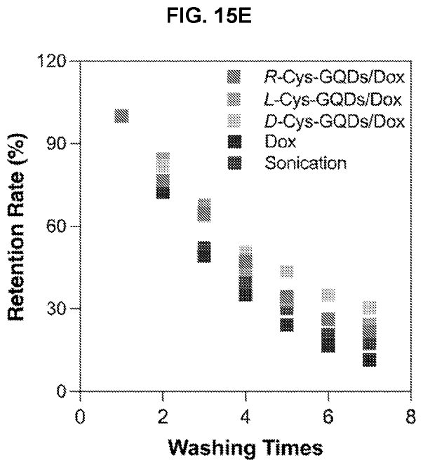

The loading efficiency of D-Cys-GQDs/Dox complex into sEVs at the optimized D-Cys-GQD concentration of 15 μM was investigated. For all the drug loading tests, D-Cys-GQDs/Dox (15 μM/200 μM) complexes were incubated with 3T3 sEVs (1.0×10⁹ particles/mL) at room temperature under a static condition. The incubated solution was then washed with 1×PBS buffer under the hold of a 100 kDa cellulose centrifugal filter for multiple times to remove excessive D-Cys-GQDs/Dox complex. The change of the fluorescence intensity of the incubated solution was monitored at each washing steps. The intensity of fluorescence decreased as the washing steps increased (FIG. 15A-D). The fluorescent intensity of D-Cys-GQDs/Dox loaded sEVs decreased moderately and kept the retention rate of 30.5% (FIG. 15E), indicating the strong interaction between D-Cys-GQDs/Dox and sEVs. In contrast, relative steep decrease and low retention rate were observed for sEVs loaded by L-Cys-GQDs/Dox (24.4%), R-Cys-GQDs/Dox (21.9%), free-Dox (11.5%) and sonication method (17.5%) (FIG. 15E).

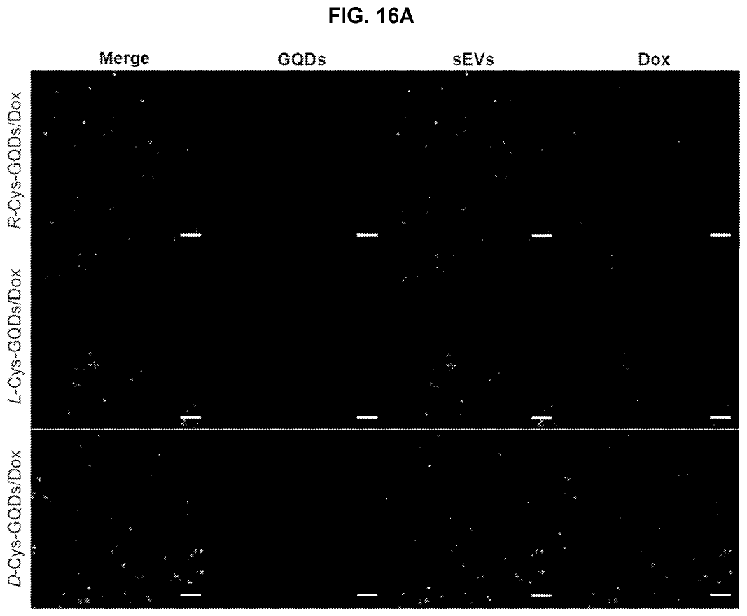

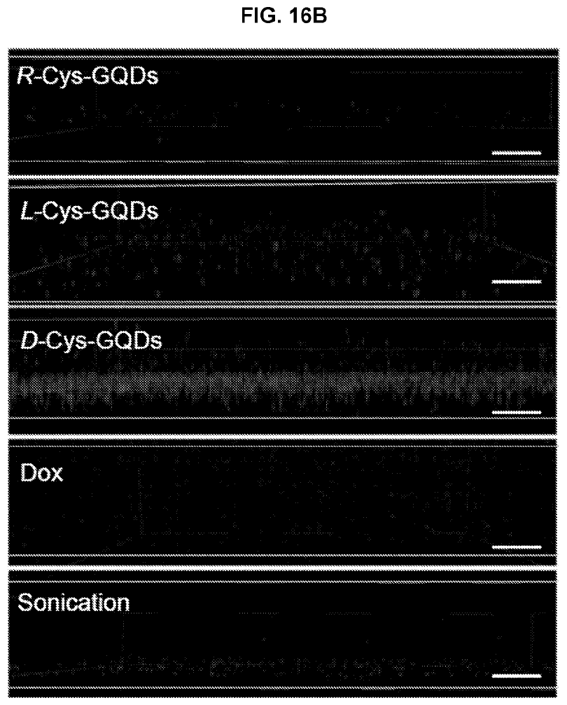

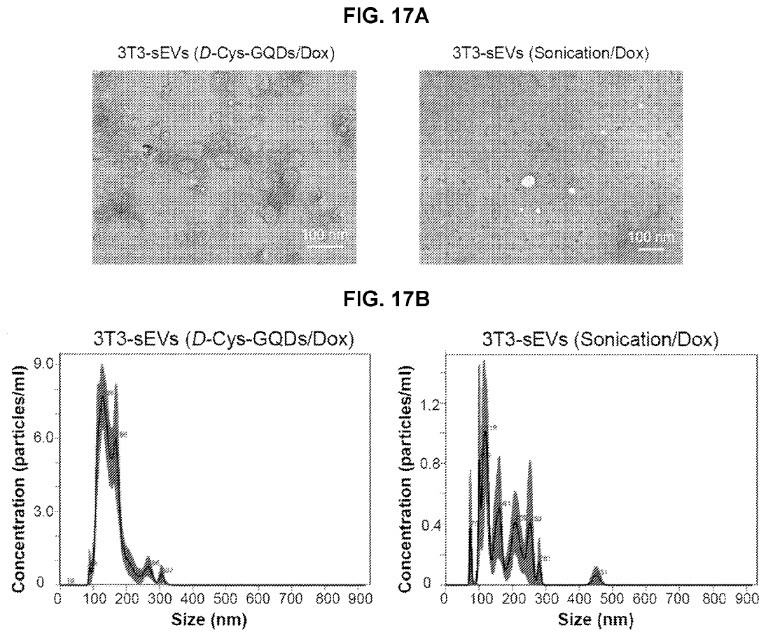

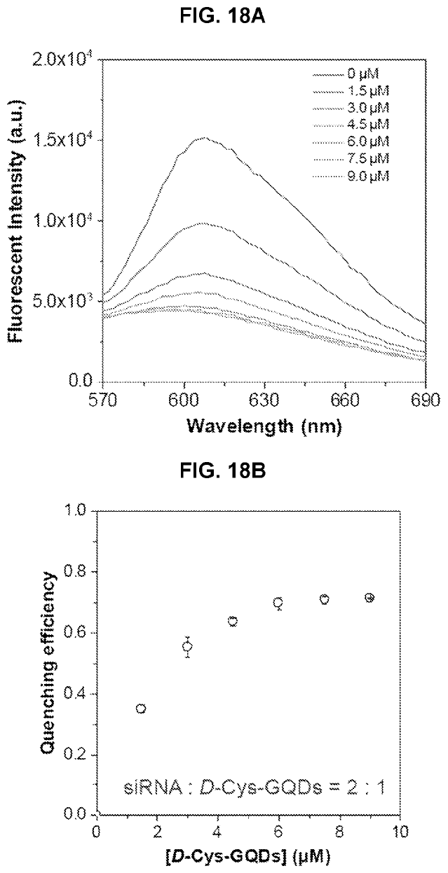

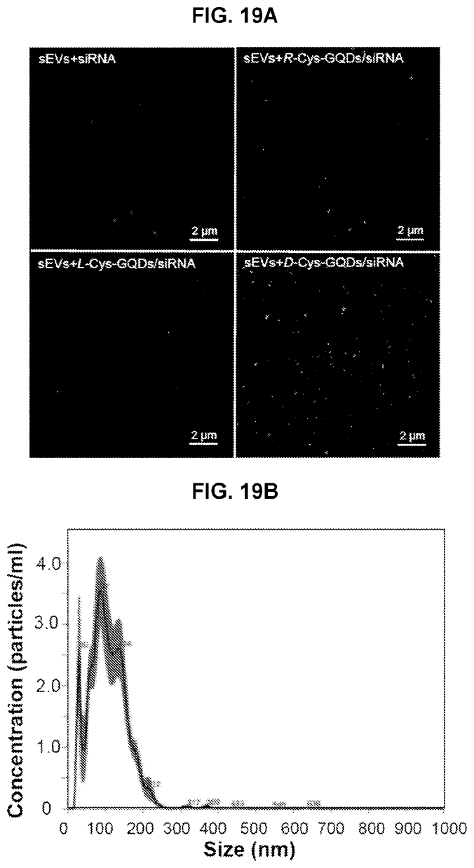

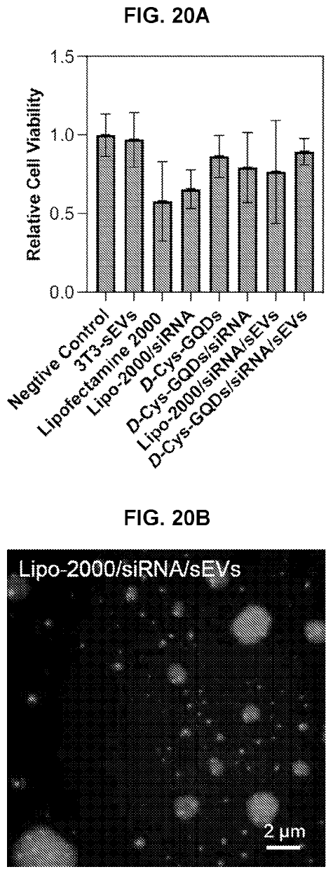

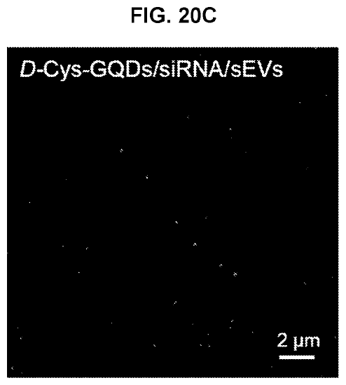

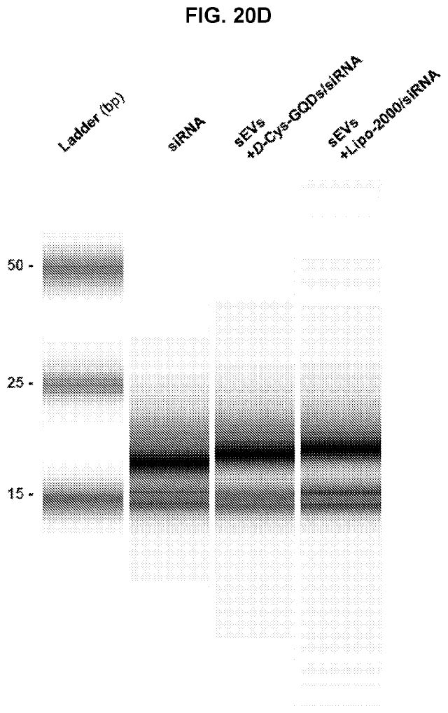

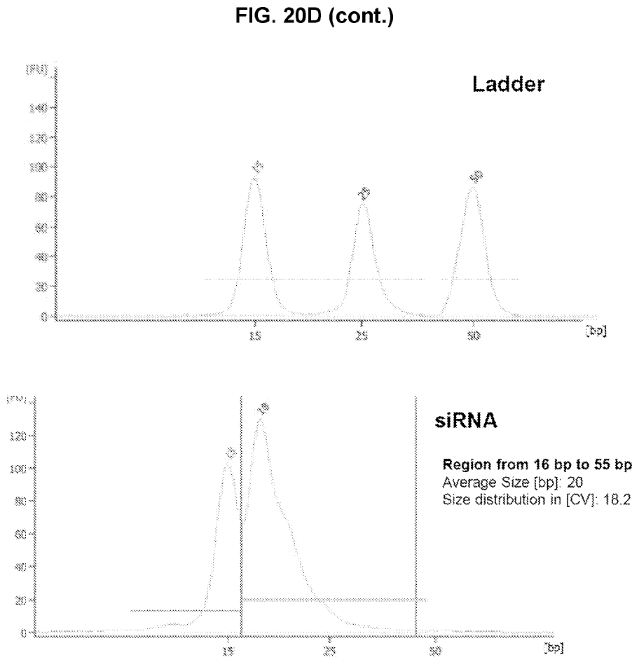

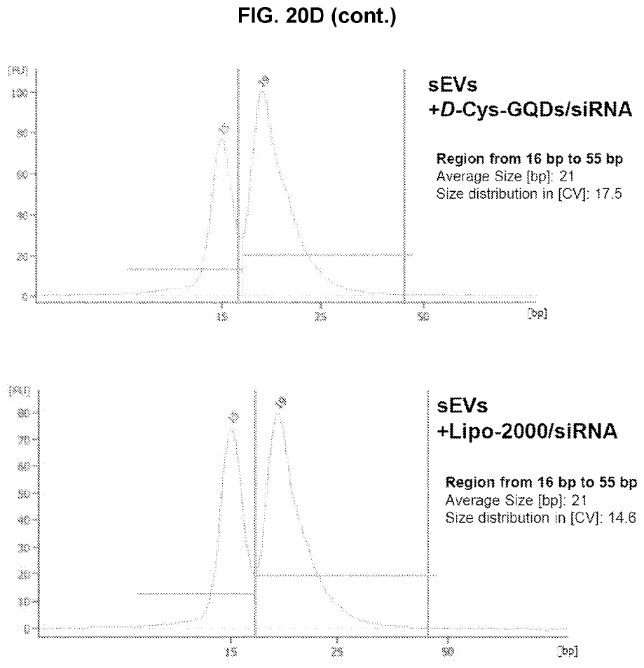

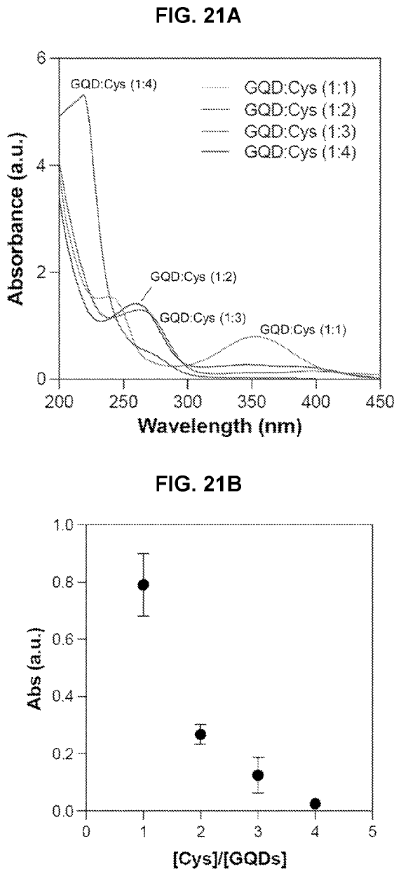

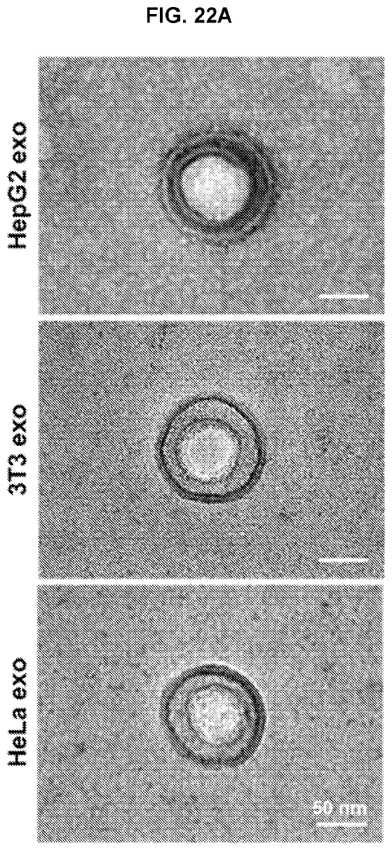

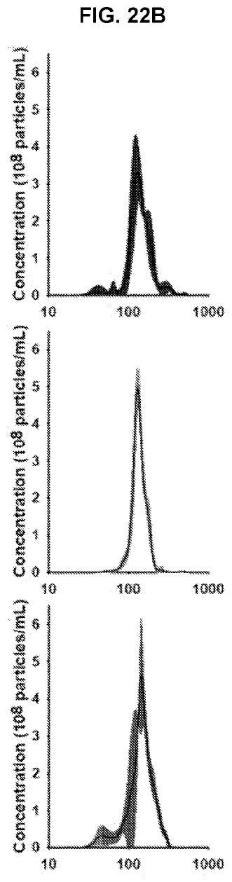

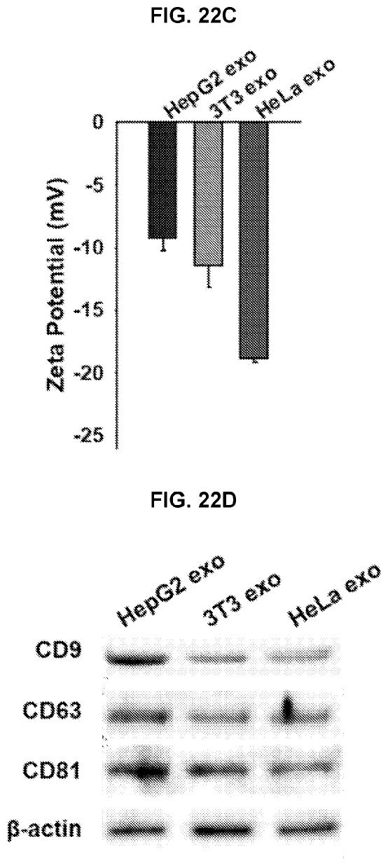

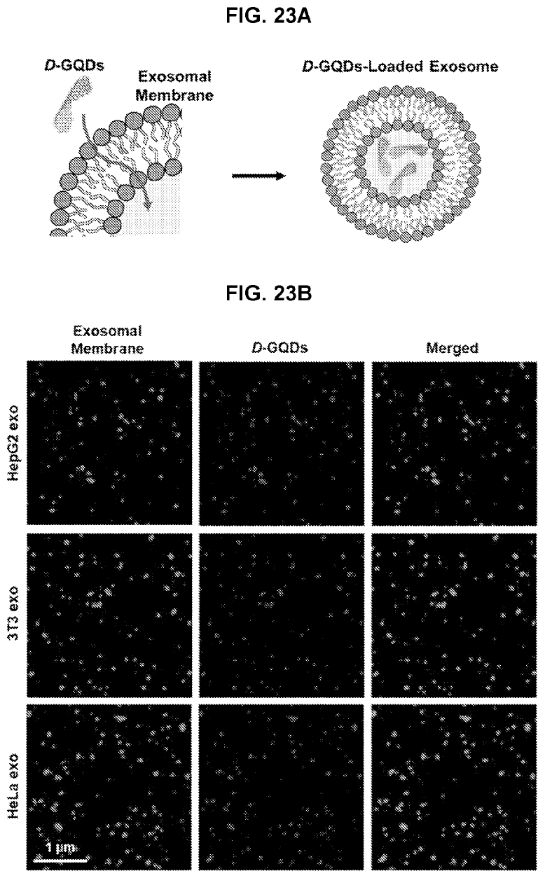

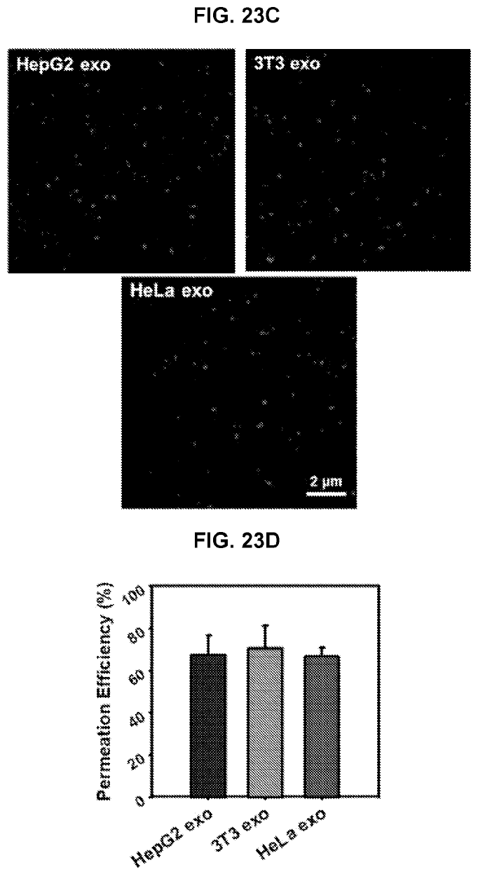

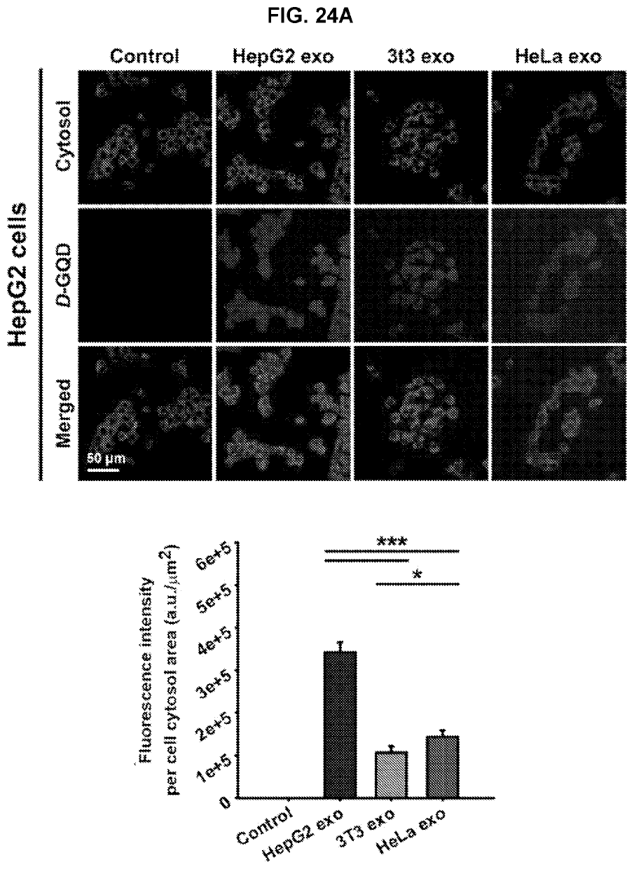

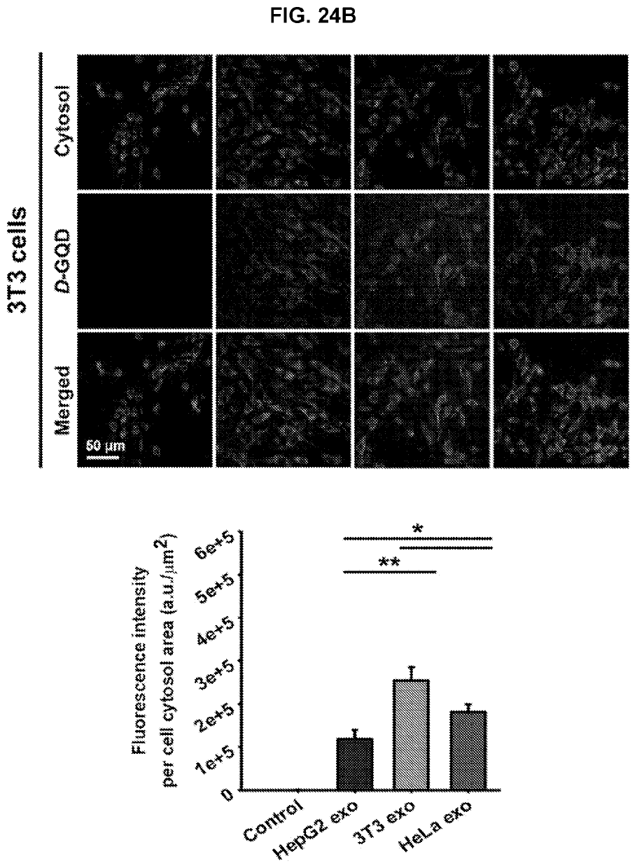

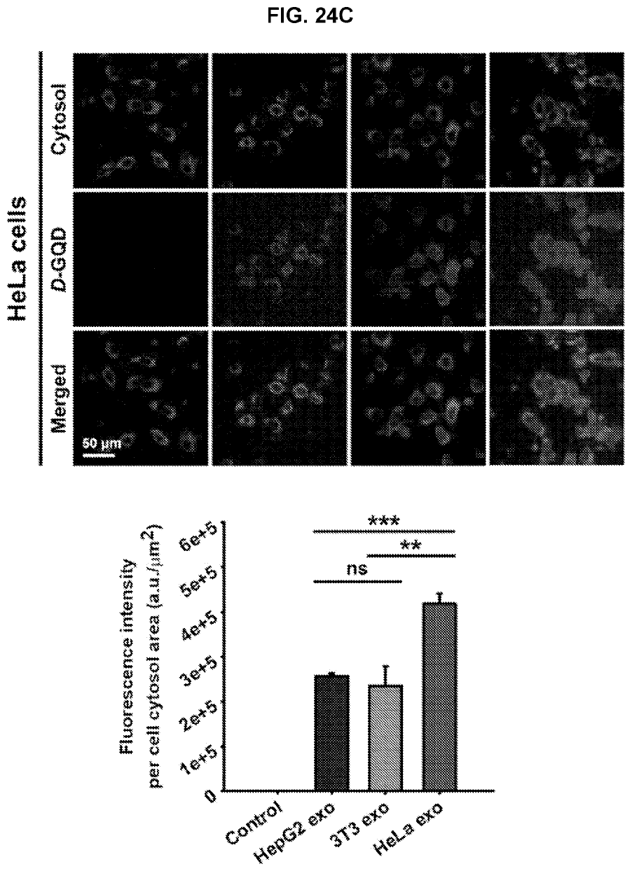

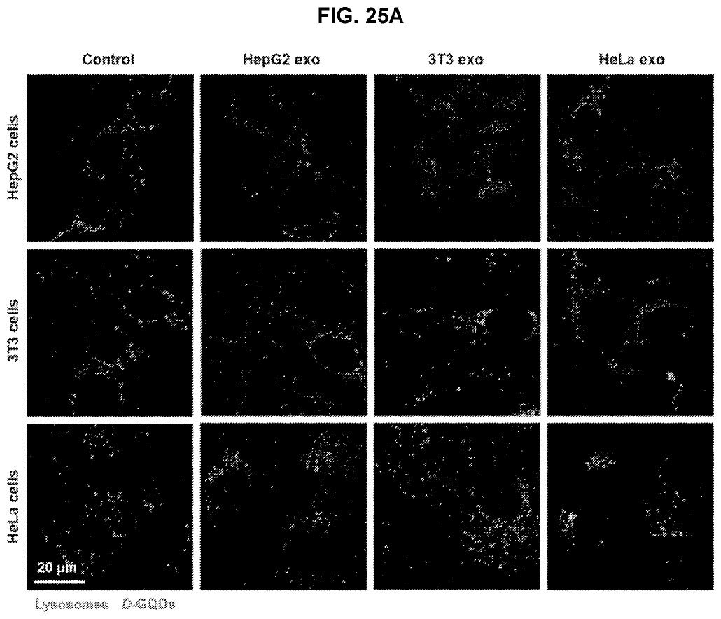

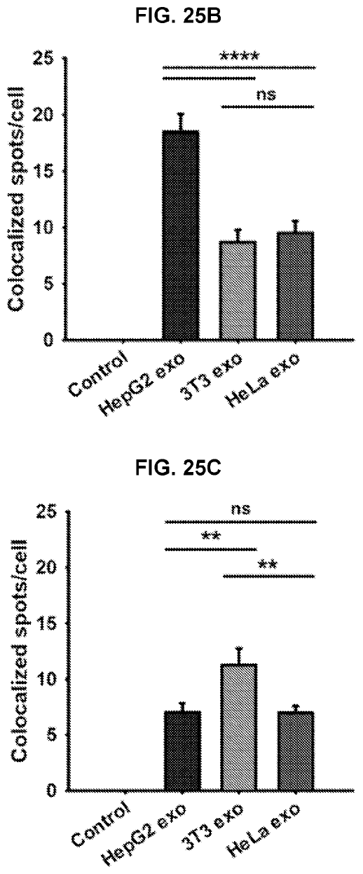

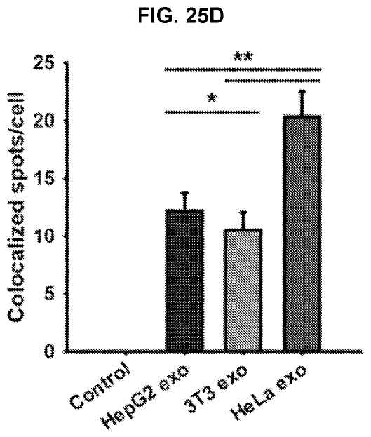

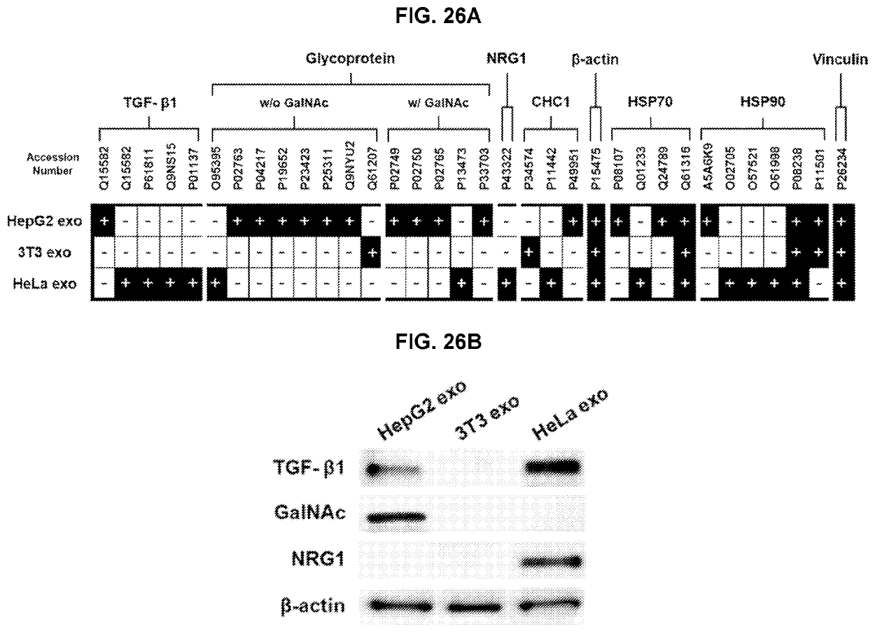

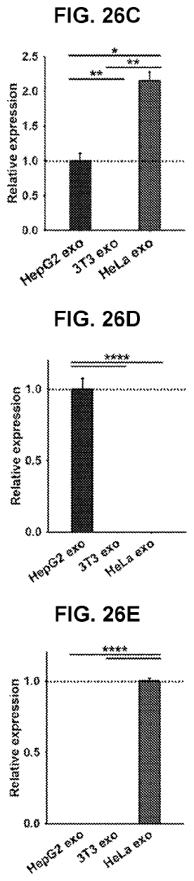

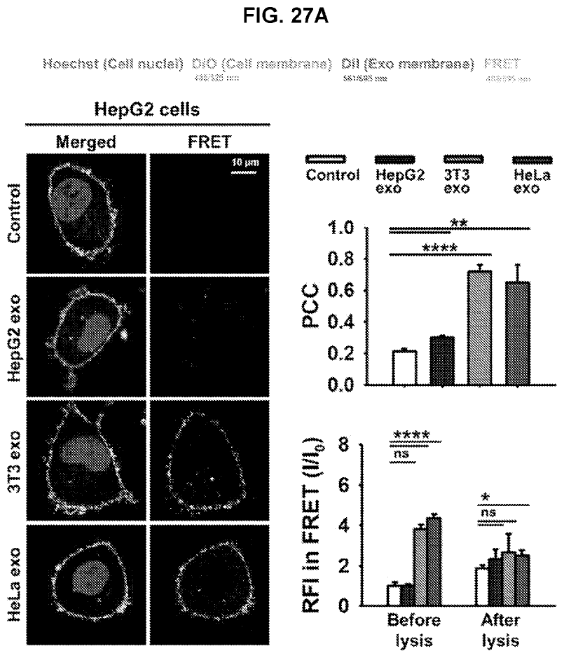

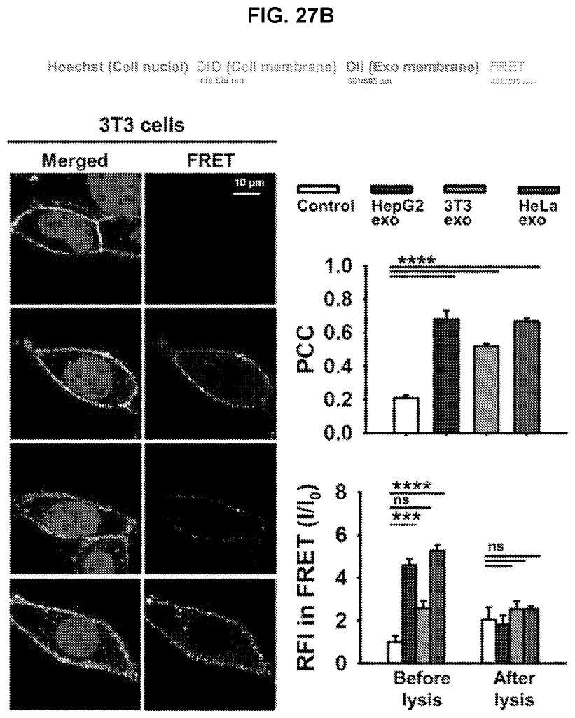

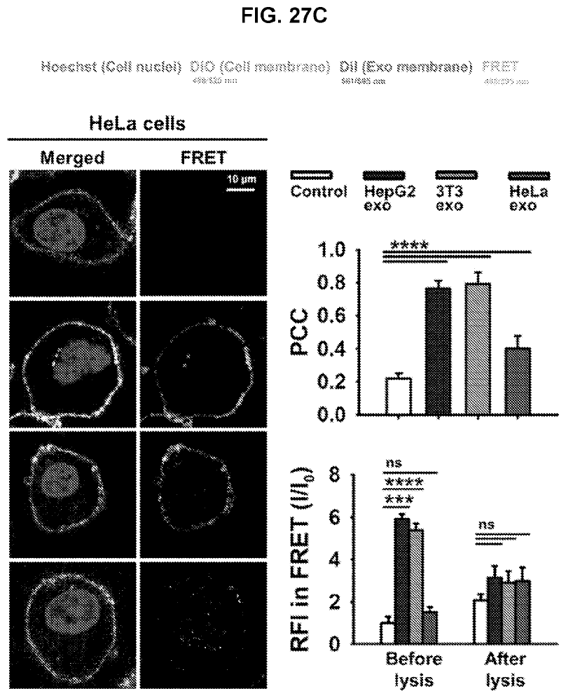

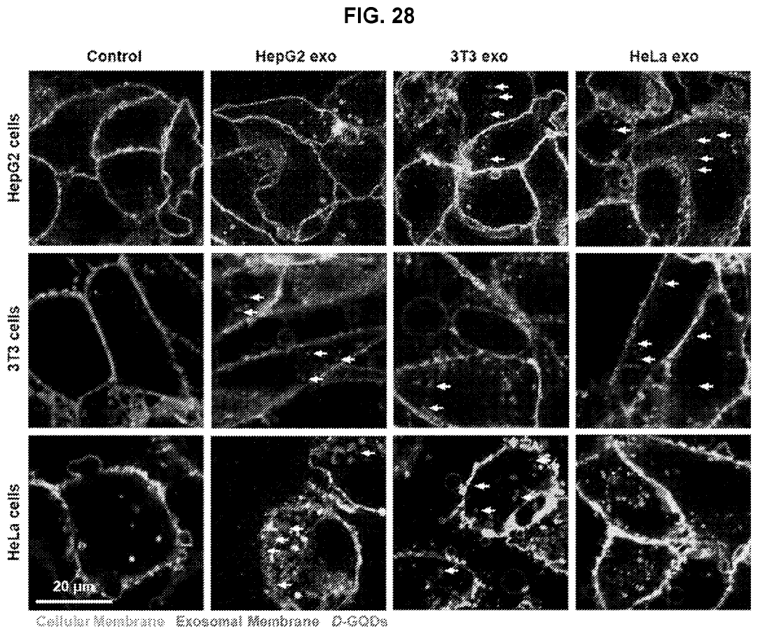

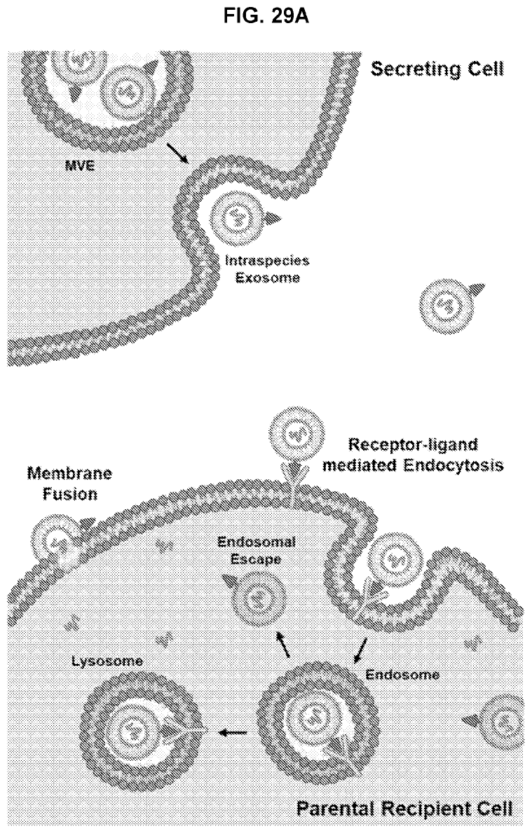

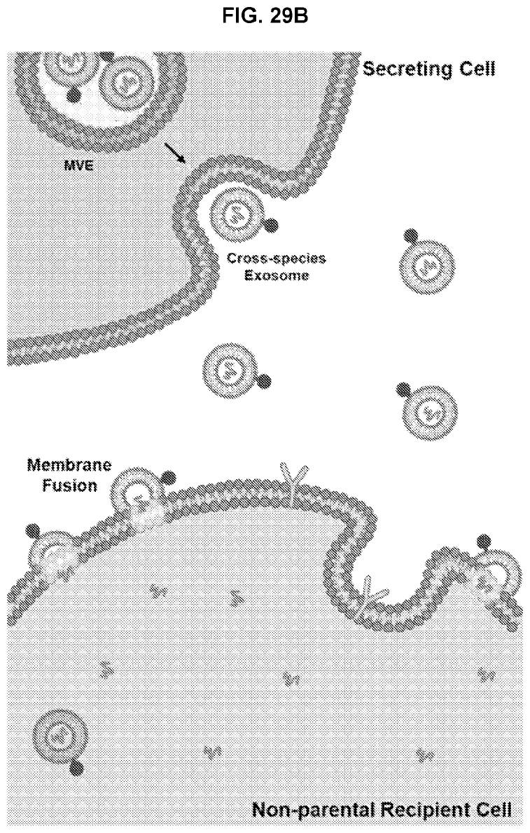

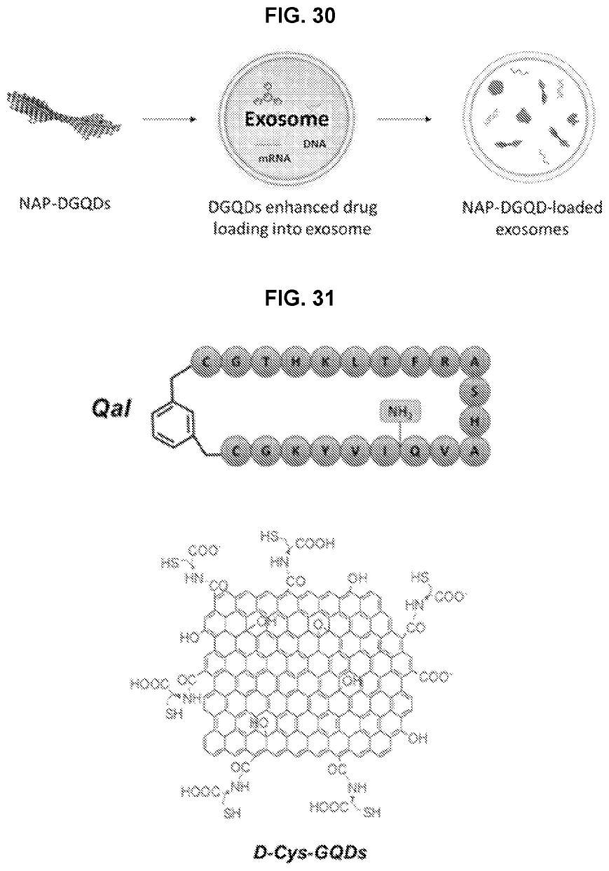

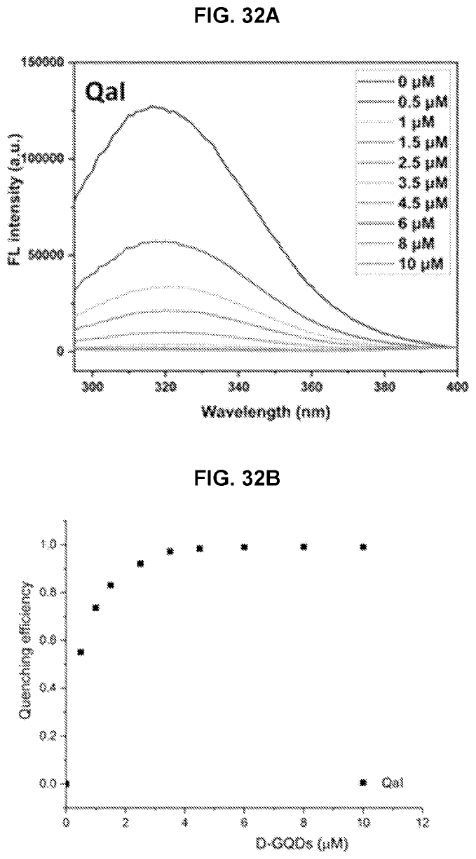

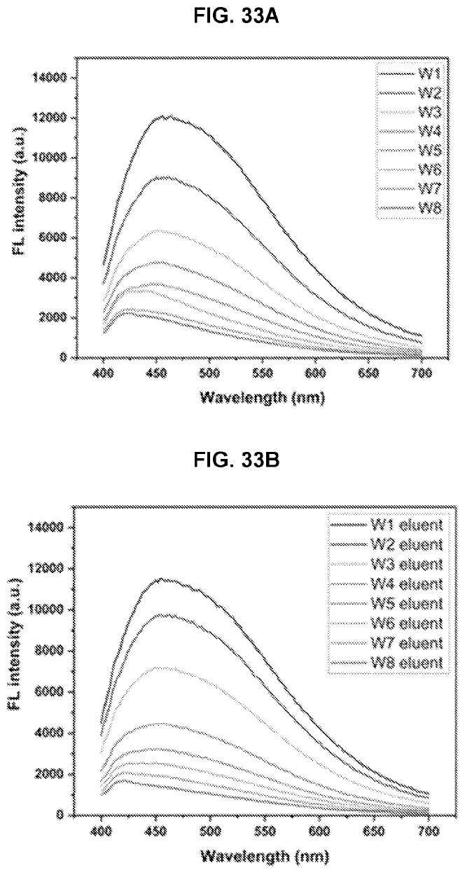

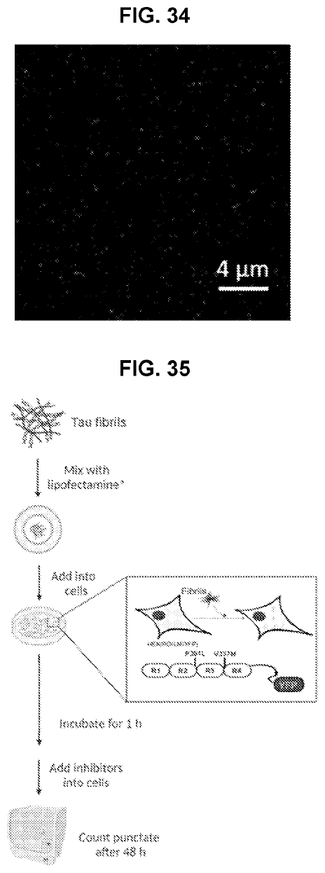

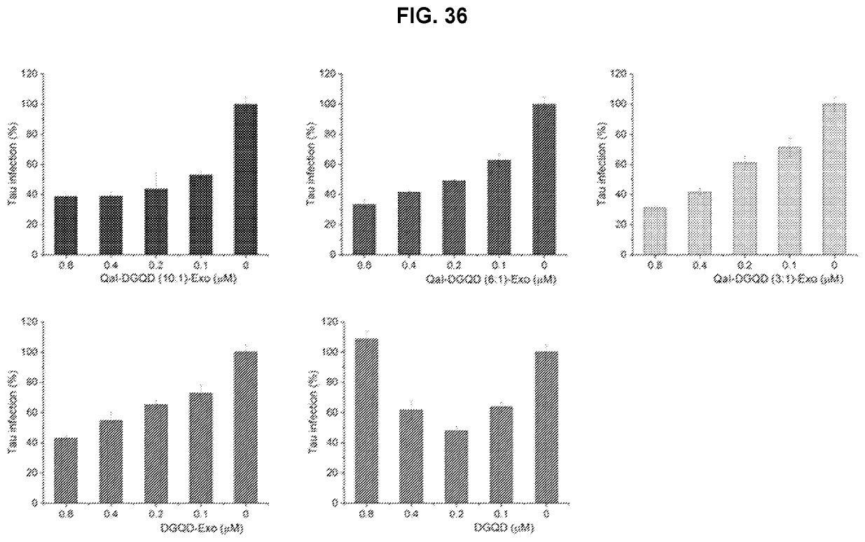

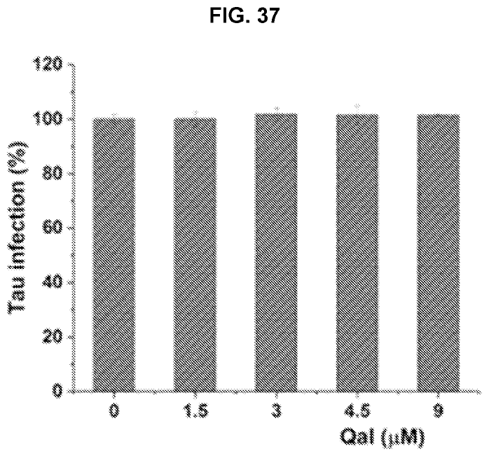

Drug loading via chiral GQDs into sEVs was further examined by confocal fluorescence microscopy by visualizing the co-localization of chiral GQDs (blue), sEVs (membrane dye in green) and the Dox (red) (FIG. 16A). Due to FRET effect, all blue signals corresponding for GQDs variants were quenched by the attachment of Dox, thus showed relatively low intensity of signal in the confocal images (FIG. 16A). Thus, the permeation of chiral-GQDs/Dox complex results were analyzed using the Dox channel (red) (FIG. 5C and FIG. 16B). Based on the definition of the encapsulation efficiency in liposome formulation, 57 drug loading efficiency of sEV is defined to be the percentage of active sEVs that successfully encapsulate drugs. Similar to the permeation efficiency of D-Cys-GQDs into sEVs, loading efficiency of D-Cys-GQDs/Dox into sEVs (66.7±9.5%) was significantly higher than that of L-Cys-GQDs/Dox (18.3±6.7%). Meanwhile, loading efficiency of R-Cys-GQDs/Dox (15.2±6.3%) is the lowest among all samples (FIG. 5D). In addition, sEVs loaded with Dox via traditional sonication approach was prepared as a control. The low loading efficiency of traditional sonication (14.5±7.9%) was reflected with relative low density of Dox signal (red) on confocal fluorescence microscopy (FIG. 5C). Most importantly, sEVs retained integrity without significant change in size after drug loading by D-Cys-GQDs. In contrast, the physical properties of sEVs were altered by sonication significantly, as shown in TEM and NTA (FIG. 17A-B). These results demonstrated that drug loading into sEVs facilitated by chiral GQDs had significantly high efficiency and maintained the integrity of sEVs.

To evaluate whether D-Cys-GQDs/Dox loaded sEVs (Exo-Dox) could be taken up by the cells, 3T3 cells were treated with Exo-Dox in vitro for 12 h and compared to the control group treated with free Dox. The presence of Dox signals (red) in the confocal images demonstrated that Dox molecules were delivered successfully into the cells and mainly accumulated in the nucleus (FIG. 5E). Meanwhile, D-Cys-GQDs (blue) were distributed mostly in the cytosol of cells, indicating the release of Dox from D-Cys-GQDs/Dox complex.

The ability of Exo-Dox to inhibit cancer cell proliferation in vitro was then analyzed. Human hepatocellular carcinoma cells (HepG2) and cervical carcinoma cells (Hela) were treated with Exo-Dox for 24 h, while cells without treatment, treated with control sEVs, and D-Cys-GQDs mixed with free Dox are negative controls. Cell viability of all samples was measured by CCK-8 assay. Exo-Dox inhibited cell proliferation by 44.2±9.2% for HepG2 cells and by 27.4±6.5% for Hela cells, comparable to free Dox (HepG2: 28.3±5.2% and Hela: 27.7±5.0%) (FIG. 5F). No significant inhibition of cell growth was observed in control samples treated with sEV and D-Cys-GQDs, indicating low or no toxicity associated with sEVs or D-Cys-GQDs. Dox-loaded sEVs by sonication method were not included in the cell viability tests due to low yield of loaded sEVs that was caused by the damage of physical structure of sEVs by sonication (FIG. 17A-B).

### Example 7

Chiral GQD-Based siRNA Loading into sEVs for Gene Therapy

siRNA therapeutics are promising treatment for viral infections, hereditary disorders, and cancers. However, it is still challenging in translational applications due to the poor intracellular uptake and limited stability of siRNA in the blood stream. When siRNA is administered intravenously, it is readily digested by nucleases and largely cleared from the kidney glomeruli before reaching the diseased organs. sEVs have been invested as nanocarriers for siRNA encapsulation to overcome this challenge due to the high stability in circulation and efficient cellular uptake compared with liposome. However, the efficacy of siRNA loading in the sEV is relatively low due to these nucleotides being relatively large and cannot diffuse into the sEV spontaneously. Moreover, current methods, such as the usage of transfection reagents and viral transduction-base strategies, may affect the function of sEVs and the pathogenicity and teratogenicity of the viruses may be preserved and inherited in sEVs, resulting in safety risks.

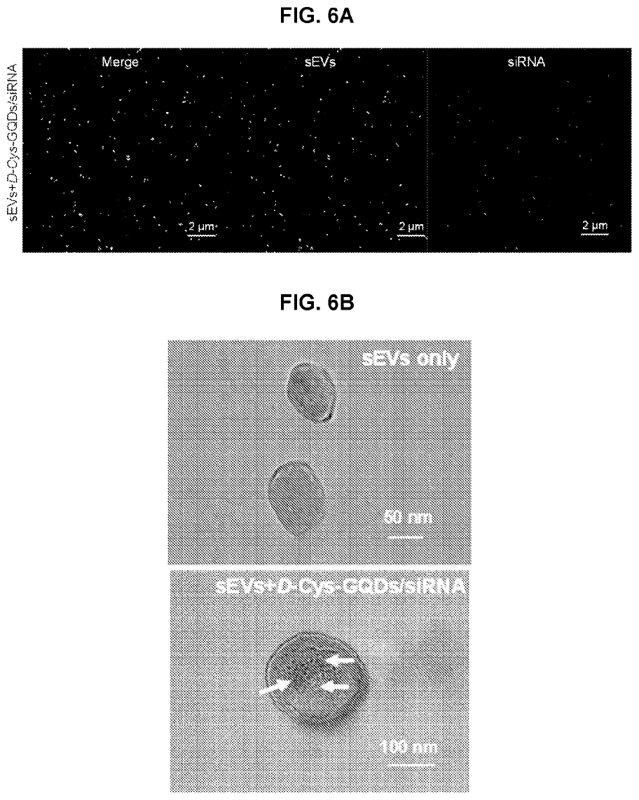

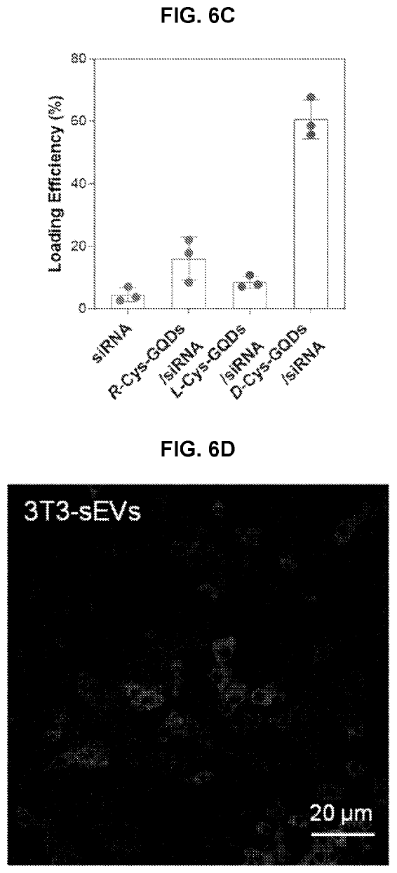

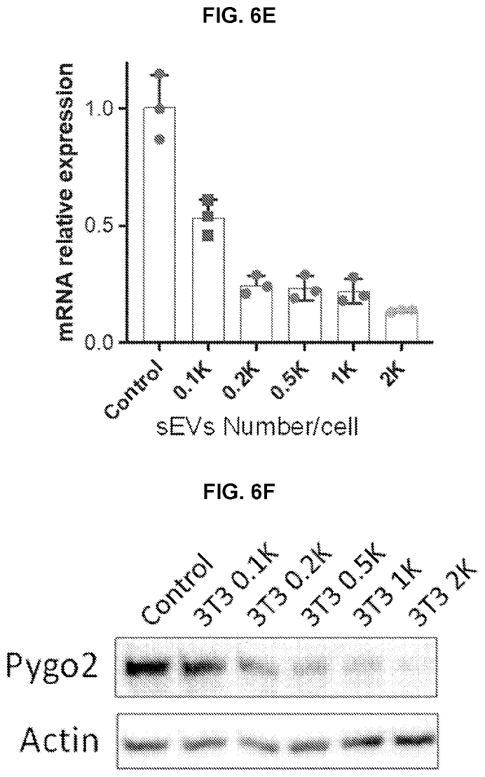

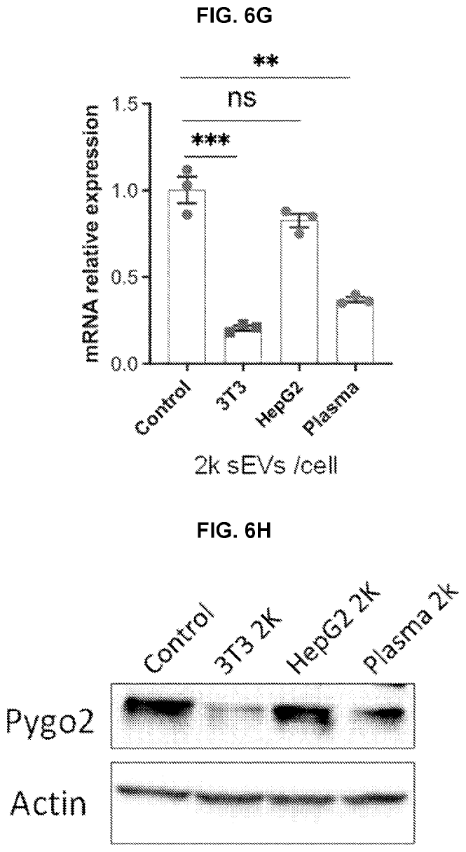

Similar to enhanced Dox loading into sEVs through D-Cys-GQDs, D-Cys-GQDs were utilized to load the siRNA into 3T3 sEVs. The aromatic surface of GQDs can offer an excellent capability to immobilize genomic substant drugs by π-π stacking. For demonstration of siRNA loading, a nucleic acid sequence (GUGCAAUGAGGGACCAGUA; SEQ ID NO: 1) labeled with red dye ROX (carboxy-X-rhodamine) was tested first. The density of the siRNA on the D-Cys-GQDs surface was determined as 2 siRNAs per GOD by the same FRET assay (FIG. 18B) as the method for Dox. The optimized condition for loading was chiral-Cys-GQDs/siRNA complex (15 μM/30 μM) determined by the saturation point of siRNA attachment on D-Cys-GQDs for the following loading investigation. The loading of D-Cys-GQDs/siRNA in sEVs was confirmed with confocal fluorescence microscopy by the colocalization signals of the labeled siRNA (red) and sEV (green, Cellmask green plasma membrane stain) (FIG. 6A). Furthermore, the D-Cys-GQDs/siRNA complex loaded sEV was visualized through TEM to confirm the successful loading. TEM images showed colocalization of D-Cys-GQDs/siRNA and sEVs with black dots in sight of individual sEV compared with the bare one (FIG. 6B). The loading efficiencies were analyzed using the same method as previous sessions based on the red signal of siRNA (FIG. 19A). Compared with free siRNA that rarely entered sEVs, the loading efficiencies of D-Cys-GQDs/siRNA, R-Cys-GQDs/siRNA and L-Cys-GQDs/siRNA were 64.1±16.5%%, 17.8±7.4%, and 7.1±4.2% (FIG. 6C and FIG. 19A). In addition, the size of siRNA-loaded sEVs remained similar to unloaded sEVs shown in NTA (FIG. 19B). The results demonstrated that siRNAs were successfully loaded in the sEV by chiral GQDs, and the sEVs remained integrity after the loading procedure.

To determine whether siRNA loaded sEVs by D-Cys-GQDs are effective and efficient for cancer treatment, the newly identified prostate cancer oncogene, Pygo2, was chosen as a target. Pygo2 is a chromatin effector and has been reported to have overexpression in prostate, ovarian, breast, cervical, hepatic, lung, intestinal, and brain cancers. Pygo2 was also recently found to play an essential role in immunosuppressive tumor microenvironment regulation and inducing the resistance of prostate cancer to immunotherapy. Therefore, targeting Pygo2 has very good implication in clinical treatment for many types of cancer. The knock-down of the Pygo2 (Pygopus homolog 2) gene using commercially available Pygo2 siRNA was previously demonstrated. Pygo2 has good efficiency on silencing and has been used for gene delivery applications. Meanwhile, DU145 is a widely used human prostate cancer cell line with high expression of Pygo2. Thus, D-Cys-GQDs/siRNA loaded sEVs using DU145 cell culture in vitro was assessed. First, the uptake of the isolated 3T3 sEVs by the DU145 cell line was tested. A strong fluorescent signal (blue: GQDs) was observed in the cytoplasm of DU145 cells incubated with D-Cys-GQDs loaded (3T3) sEVs for 48 hours (FIG. 6D). Efficiencies of gene knock-down by D-Cys-GQDs/siRNA loaded sEVs were confirmed with qRT-PCR and Western blotting. For 3T3 sEVs (100 to 2000 Exo/cell), it showed significantly reduction of Pygo2 mRNA levels by 45%~80% with a mean value of 82% (FIG. 6E). The expression of Pygo2 protein levels in DU145 cells were also silenced by D-Cys-GQDs/siRNA loaded (3T3) sEVs by 22%~91%, compared to the control group (FIG. 6F). In addition, D-Cys-GQDs/siRNA induced a dose-dependent decrease for both mRNA and protein expressions. To reflect loading reliability of D-Cys-GQDs, sEVs isolated from two other sources, HepG2 cell culture medium and healthy human plasma, were tested as controls. Similarly, D-Cys-GQDs/siRNA loaded plasma sEVs reduced expression of mRNA by 63.3±7.8% (FIG. 6G) and expression of protein by 50% (FIG. 6H). HepG2 sEVs showed no obvious reduction of mRNA and Pygo2 protein, which contributed to the fact that the sEV uptake capabilities were different depending on the recipient cell types. An overall 60%-80% knock-down of the target gene and higher than 60% inhibition of Pygo2 protein, indicating the efficacy of D-Cys-GQDs/siRNA loaded sEVs to the target cells. Overall, D-Cys-GQDs can facilitate loading genes such as siRNA into sEVs without membrane damage of sEVs and the loaded sEVs can successfully mediate silencing of target genes with high efficiency.

To compare the disclosed loading strategy with other known methods in terms of cytotoxicity and RNA stability, siRNA was loaded into sEVs using D-Cys-GQDs and a common transfection reagent, Lipofectamine 2000 (Lipo-2000). First, cytotoxicity of siRNA-loaded sEVs was tested on HepG2 cells. HepG2 cells were incubated with siRNA-loaded sEVs (10,000 particles/cell) for 24 h. The responses of cells to siRNA-loaded sEVs were monitored by a CCK-8 assay (FIG. 20A). siRNA-loaded 3T3-sEVs by D-Cys-GQDs exhibited no inhibition of cell proliferation (cell viability: >95%), while siRNA-loaded 3T3-sEVs by Lipo-2000 exhibited slightly higher toxicity in HepG2 cells (cell viability: 76%), which could potentially be attributed to the fusion of cationic lipids with sEVs. Moreover, confocal fluorescent images of the sEV solution (FIG. 20B) after transfection of a ROX-labeled siRNA by Lipo-2000 suggested that the siRNA and Lipo-2000 formed large aggregates that were not able to fuse to the sEV membranes or enter the sEVs. In contrast, the disclosed chiral-GOD-assisted siRNA loading strategy produced excellent dispersion after the loading and the sEVs retained their integrity after siRNA loading by D-Cys-GQDs (FIG. 20C). In addition, controls were tested, including 3T3-sEVs (10,000 particles/cell), Lipo-2000 (1 μL, unknown concentration, Thermo Fisher Scientific), Lipo-2000/siRNA (1 μL/20 μmol), D-Cys-GQDs (15 nM), and D-Cys-GQDs/siRNA (15 nM/30 nM). Bare 3T3-sEVs displayed no cytotoxicity toward HepG2 cells. Meanwhile, inhibition of cell growth was not observed in the control samples treated with D-Cys-GQDs and D-Cys-GQDs/siRNA, indicating low or no toxicity associated with D-Cys-GQDs and their complex of D-Cys-GQDs/siRNA. Lipo-2000 and Lipo-2000-encapsulated siRNA showed a slight toxicity in HepG2 cells (cell viability: 60-70%), which was likely due to the side effect of cationic lipid on caspase activation dependent signaling pathway and mitochondrial dysfunction. siRNA stability was also evaluated by the Bioanalyzer system (FIG. 20D), which is a tool commonly used to assess the quality of RNA samples. The Bioanalyzer system uses microfluidic electrophoresis to separate RNA molecules based on size and generates a digital electropherogram that provides valuable information on the quality of RNA samples before downstream applications. The siRNA loading strategies using chiral GQDs were found to exhibit a consistent size distribution of siRNA (Pygo2) that was comparable to the siRNA control group, as well as the traditional transfection reagent. Overall, the chiral GQD-assisted loading strategy exhibited lower cell toxicity and similar RNA stability compared to the common transfection agent loading method.

This work investigated the permeability of chiral GQDs into sEVs through a lipid membrane, and the ability of these chiral GQDs to passively load drugs into sEVs (FIG. 1A). D-GQDs derived from D-cysteine were found to have a stronger tendency (52.4±8.2% of permeation efficiency) to permeate into sEVs than pristine GQDs and L-GQDs, and the permeability of D-GQDs could further be improved to 85% after optimization. Taking advantage of chiral interactions between chiral nanoparticles and biological lipid membranes, and the synergistic effect of the physical surface of chiral GQDs, the hydrophobic chemotherapy drug doxorubicin (Dox) and a large biological siRNA drug were successfully loaded into sEVs. The Dox/siRNA-loaded sEVs exhibited effective killing of cancer cells, with significant knock-down of the targeted gene and inhibited expression of relative protein levels. This work indicated that passive drug loading into sEVs enhanced using chiral GQDs is a robust (drug-agnostic) and scalable loading method that requires minimal tuning. Therefore, the developed chiral GQD-enhanced drug loading technology provides a promising platform for loading therapeutic lipid-based carriers.

In summary, an exogenous drug-agnostic chiral GOD sEV-loading platform was developed, based on chirality matching with sEV lipid bilayers. Both hydrophobic and hydrophilic chemical and biological drugs can be functionalized or adsorbed onto GQDs by π-π stacking and van der Waals interactions. By tuning the ligands and GQD size to optimize its chirality, significantly high drug loading efficiency was demonstrated for Dox and siRNA into sEVs by D-Cys-GQDs at an optimal concentration, compared to other reported sEV loading techniques. The sEVs loaded with drugs using the D-Cys-GQDs were shown to be effective in killing cancer cells, knocking-down the target gene, and inhibiting mRNA and relative protein expression levels. Thus, chiral GQD-enhanced drug loading is a promising generic and scalable drug loading technique that can enable high-throughput production of therapeutic sEVs and other lipid-based carriers for various commercial, research, and clinical applications.

### Example 8

### Fluorescent Chiral GQDs to Study Origin-Dependent Exosome Uptake and Cargo Release Materials and Methods

Carbon nanofibers (719803) and PKH26 (MINI26-1KT) were purchased from Sigma-Aldrich (MO, USA). Sulfuric acid (BDH3068-500MLP), Nitric acid (BDH3044-500MLPC), and Fetal bovine serum (FBS; 1300-500) were purchased from VWR (PA, USA). Dialysis membrane tubing (MWCO: 1 kD; 20060186) was purchased from Spectrum Chemical Manufacturing Company (NJ, USA). 1-ethyl-3-(3-dimethyl-aminopropyl) carbodiimide (EDC; 22980), Vybrant™ Multicolor Cell-Labeling Kit (DiO, DiI; V22889), and Gibco™ Trypsin-EDTA (25200072) were purchased from Thermofisher Scientific (MA, USA). N-hydroxysuccinimide sodium salt (Sulfo-NHS; 56485) and D-cysteine (A110205-011) were purchased from AmBeed (IL, USA). Nitrocellulose membrane (1662807) and Clarity Max Western Enhanced Chemiluminescence (ECL) Substrate (1705060) were purchased from Bio-Rad (CA, USA). RIPA Buffer (9806), anti-mouse Horseradish Peroxidase (HRP)-linked secondary antibody (7076), and anti-rabbit HRP-linked secondary antibody (7074) were purchased from Cell Signaling Technology (MA, USA). Anti-CD9 antibody, anti-CD63 antibody, anti-CD81 antibody, and anti-rabbit HRP-linked secondary antibody (EXOAB-KIT-1) were purchased from System Biosciences (CA, USA). Anti-beta-actin antibody (sc-47778), anti-neuregulin1 antibody (sc-393006), and anti-GalNAc antibody (sc-393370) were purchased from Santa Cruz Biotechnology (TX, USA). Anti-TGF-beta1 antibody (21898-1-AP) was purchased from Proteintech (IL, USA). LysoView™ 594 (70084), Hoechst 33342 (40046), and CellBrite Green Cytoplasmic Membrane Dye (30021) were purchased from Biotium (CA, USA). CytoTrace™ Red CMTPX (22015) was purchased from AAT Bioquest (CA, USA). Minimum essential medium eagle (MEM; 10-010-CV) and Phosphate-buffered saline (PBS; 21-040-CM) were purchased from Corning (NY, USA). Antibiotic antimycotic (15240096) was purchased from Fisher Scientific (MA, USA). 4% Paraformaldehyde (PFA; 15735-50S) and UranyLess (22409) were purchased from Electron Microscopy Sciences (PA, USA). Tween-20 (BTNM-0080) was purchased from G-Biosciences (MO, USA).

Transmission electron microscopy (TEM; JEOL 2011, JEOL Ltd., Tokyo, Japan) was used to confirm nanostructures. Zeta potential values were characterized by Zetasizer (Malvern Zetasizer Nano ZS, Malvern Instrument Ltd., Worcestershire, UK). Nanoparticle Tracking Analysis (NTA; NanoSight LM10 system, Malvern Instrument Ltd., Worcestershire, UK) was used to measure the size distribution and concentration of nanoparticles. Confocal Laser Scanning Microscopy (CLSM; A1R-MP Laser Scanning Confocal Microscopy, Nikon, Tokyo, Japan) was operated to observe the cellular uptake and penetration of chiral GQDs into sEVs. Fluorescence was measured by a plate reader (Infinite 200 PRO, Tecan, Minnedorf, Switzerland). Circular dichroism (CD) spectrometer (Jasco J-1700 Spectrometer, Jasco International Company, MD, USA) was used to measure the absorbance of polarized light were measured. Mass spectrometer (MS; Thermo Q-Exactive HF, Thermo Fisher, MA, USA) was used to identify the proteins. Western blot images for protein immunodetection were acquired using a bioimaging system (C400 Bioanalytical Imager, Azure Biosystems, CA, USA). Humidified incubator (MCO-15AC, Osaka, Japan) was used to maintain and incubate all cell lines used in the study.

### GQD Synthesis and Chiral Functionalization

The GQDs were synthesized using a modified protocol based on previous reports as follows: a 40 mL mixture of sulfuric acid and nitric acid (3:1, v/v) and 0.4 g of carbon nanofibers were sonicated for 2 h. After being mechanically stirred for 8 h at room temperature, the mixture was heated to 120° C. and allowed to react continuously for 10 h. Then, the mixture was cooled, diluted with ice-cold deionized water, and pH was adjusted to 8 by adding sodium hydroxide. After 3 d of dialysis for purification, GQDs were obtained with a final concentration of 1 mg/mL. To functionalize the GQDs with chirality, the carboxylic group of GQDs was connected to the amine group of D-cysteine by EDC/NHS coupling reaction as follows: 20 μL of EDC (100 mM) was added to 2 mL of GQDs (100 μM) solution, followed by 10 min of stirring. Then, 40 μL of Sulfo-NHS (100 mM) was added and sonicated for 40 min under an ice-water bath. In order to remove excess EDC and Sulfo-NHS, the mixture was filtered with a 1 kDa centrifuge tube and rinsed three times. Finally, 40 μL of D-cysteine (100 mM) was added and the mixture was stirred for 2 h. The excess D-cysteine was removed by a dialysis membrane. The chiroptical activity was measured using a CD spectrometer, and the fluorescence property was measured using a plate reader. The morphology of chiral GQDs was observed using TEM. The SigmaPlot 10.0 (Systat Software Inc., CA, USA) was used to generate plots of the analyzed data.

### Cell Culture

Human hepatoma cells (HepG2), mouse fibroblast cells (3T3), and human cervical adenocarcinoma cells (HeLa) were cultured in MEM supplemented with 10% FBS and 1% Antibiotic-Antimyotic in a humidified incubator at 37° C. with 5% CO₂. Cells were washed with 1×PBS, trypsinized with trypsin-EDTA for passages, and incubated at least 1 d before any experiments were conducted. Cells were stained with several dyes for imaging under CLSM: CytoTrace Red CMTPX dye for cytosol area staining; CellBrite Green Cytoplasmic Membrane dye and DiO dye for membrane staining; LysoView 594 dye for lysosome staining; and Hoechst 33342 dye for nuclei staining. Cells were fixed with 4% PFA for 15 min before imaging.

Isolation and Characterization of sEVs

Once HepG2, 3T3, and HeLa cells covered 70-80% of the flask, cell-culture medium (CM; MEM with 10% FBS and 1% Antibiotic-Antimyotic) was changed into serum-free CM after washing three times with 1×PBS. The serum-free CM was collected after 48 h. Then, the serum-free CM was filtered with Pore Size 0.22 μm Vacuum Filtration Systems (10040-460; VWR, PA, USA) to remove undesired large debris. The filtered CM was filtered through 0.05 μm pore size hydrophilic membranes (111103; Cytiva, MA, USA) under mild-vacuum filtration conditions for 2 d. Then, the CM was washed with 1×PBS buffer and concentrated in 100 kDa ultrafiltration centrifugal devices (Spin-X UF Concentrator; Corning, NY, USA). With the negative staining by UranyLess, the morphology and nanostructure of sEVs were observed with TEM and the size distribution of 55-65 sEVs was analyzed using ImageJ software. NTA measurements were performed using diluted sEVs (200 times dilution; 5 μL in 1 mL of PBS buffer) and analyzed with NTA 3.3 analytical software suite. Surface charge of sEVs was measured by a zetasizer. To activate the disposable zeta potential cuvette (DTS1070, Malvern Instrument Ltd., Worcestershire, UK), it was rinsed once with 100% ethanol and washed twice with distilled water. sEVs diluted into 2-3×10⁶ sEVs/mL were loaded in the cuvette and 5 measurements per sample were made with 30-100 autoruns for each using the Hückel model. sEV marker expressions (CD9, CD63, and CD81) were validated through western blot sEV membranes were stained with PKH26 and Dil dye for imaging with CLSM. All characterization procedures were performed at room temperature. The SigmaPlot 10.0 was used to generate plots of the analyzed data.

Permeation of Chiral GQDs into Exosomes (sEVs)

D-GQD concentrations of 12 and 15 μM were incubated with 1×10⁹ particles/mL exosomes at room temperature with protection from light. The incubated mixture was then washed with 1×PBS buffer in a 100 kDa cellulose centrifugal filter 4 times to remove excessive D-GQDs. The permeation efficiency of D-GQDs into exosomes (D-GQDs/exo) was indirectly determined by statistical analysis using the following formula:

$$Permeation efficiency ( \% ) = \frac{\sum ( TFEPs - CFEPs )}{4 \times TEC} \times \frac{18 mm \times 18 mm}{150 µm \times 150 µm} \times \frac{1}{5 µL} \times 100 ,$$

where TEC is the total exosome concentration (partides/mL) that is measured by NTA.

5 μL of D-GQDs/exo was loaded on the Poly-L-Lysine coated slides (63410; Electron Microscopy Sciences, PA, USA) and was fully covered with 18 mm×18 mm square cover glasses (2845-18; Corning, NY, USA). The covered area was scanned under a 100× objective with a normal 150 μm×150 μm field size. Each scan contained around 25 z-stack images (step size of 0.13 μm) of appearing/disappearing blue-fluorescent dots. Four z-stack images were captured through random domains. The z-stack images were set manually to adjust the thresholds and match the diameter size range of exosomes (40-140 nm). Then, by counting the blue-fluorescent lit-up D-GQDs/exo (due to permeation) using ImageJ software, the total fluorescent exosome particles (TFEPs) could be measured. Colocalized fluorescent exosome particles (CFEPs) of z-stack images, between successive image sets, were counted via ImageJ software with JACOPx Plugin. The total exosome concentration (TEC) was measured by NTA in particles/mL. For additional validation, the permeated D-GQDs were quantified by fluorometric way. The assessment of fluorescent recovery was conducted before and after lysis of exosomes with biocompatible surfactants, Tween-20. The samples were loaded into a 96-well black plate (3991; Corning, NY, USA), and fluorescence intensity (FI) was measured using a plate reader with excitation at 360 nm and emission at 510 nm. The SigmaPlot 10.0 was used to generate plots of the analyzed data.

### Cellular Uptake Imaging of Exosomes

Cellular uptake images were obtained by CLSM. All cells were washed out with 1×PBS and fixed with 4% PFA before imaging. To observe the cellular uptake of exosomes, in vitro experiments were conducted as follows: CytoTrace™ Red CMTPX pre-stained cells were cultured at 5×10⁴ cells per well in 8-well confocal plates (Lab-Tek II Chambered Coverglass, 155409; Thermofisher Scientific, MA, USA) and incubated overnight at 37° C. under 5% CO₂. Three different types of D-GQD/exo were treated to each well with a concentration of 0.2×10³ exosomes per cell. Then, cells were incubated for 6 h at 37° C. under 5% CO₂. To observe the lysosomal uptake of exosomes, in vitro experiments were conducted as follows: cells were cultured at 5×10⁴ cells per well in 8-well confocal plates and incubated overnight at 37° C. under 5% CO₂. Three different types of D-GQD/exo were treated to each well with a concentration of 0.2×10³ exosomes per cell. Then, cells were incubated for 4 h at 37° C. under 5% CO₂ and LysoView 594 was added for staining lysosomes in the cells. To observe the direct membrane fusion of exosomes, in vitro experiments were conducted as follows: cells were cultured at 5×10⁴ cells per well in 8-well confocal plates and incubated overnight at 37° C. under 5% CO₂. Hoechst 33342 was added for staining nuclei of the cells. Three different types of D-GQD/exo stained with Dil were treated to each well with a concentration of 0.4×10³ exosomes per cell, followed by incubation for 1 h at 37° C. under 5% CO₂. Then, cellular membranes were stained by DiO. To observe the exosomal cargo (D-GQDs) delivery of exosomes, in vitro experiments were conducted as follows: cells were cultured at 5×10⁴ cells per well in 8-well confocal plates and incubated overnight at 37° C. under 5% CO₂. Three different types of D-GQD/exo stained with PKH26 were treated to each well with a concentration of 0.2×10³ exosomes per cell. With the incubation for 6 h at 37° C. under 5% CO₂, cellular membranes were stained by CellBrite Green Cytoplasmic Membrane dye. The SigmaPlot 10.0 was used to generate plots of the analyzed data.

### Image Processing and Quantification

All CLSM imaging processing and analytical quantification used the ImageJ software. Prior to every analysis, the manual conversion of length units from pixels to μm by the scale bar on CLSM images was set. The cellular uptake of exosomes was quantified as follows: Z-stack images of each group were scanned into three z-stack-set with a step size of 0.5 μm. The cellular taken up D-GQDs, exosomal cargo markers, were quantified with the integrated intensity analysis. The RawIntDen (RID; a.u.) value which is the sum of all fluorescent intensity in the region-of-interest (ROI) was measured from the montages consisting of CLSM three z-projected images in the blue channel. The red area of cellular cytosol was converted to monochrome (black and white) and the middle (second) image of z-projection was selected for quantification. The cell cytosol area was quantified using the following equation:

$$Cell cytosol area ( {µm}^{2} ) = \frac{{RID}_{mono} ( a . u . )}{{Max}_{g} ( a . u / {µm}^{2} )} .$$

The maximum and minimum gray levels (Max_{g}, Min_{g}; a.u./μm²) reflect maximum-gray (represented as black) and minimum-gray (represented as white), respectively, and the Min_{g} is zero. Finally, the fluorescence intensity per cell cytosol area was calculated using the following equation:

$$Fluorescence intensity per cell cytosol area ( a . u . / {µm}^{2} ) = \frac{{RID}_{blue} ( a . u . )}{Cellular cytosol area ( {µm}^{2} )}$$

The lysosomal entrapment of exosomes was quantified as follows: only colocalized purple spots were selected, whereas all non-colocalized red and blue spots were deselected, via ImageJ software with JACOPx Plugin. Then, the purple-colocalized spots were counted. The total counted numbers were divided by the total cell number within scanned images.

The membrane fusion of exosomes was quantified as follows: Pearson's correlation coefficient (PCC) was calculated for the analytical quantification of colocalization between cellular and exosomal membranes. The scatter plot of the red and green channels was generated using ImageJ software with the JACOPx Plugin. Subsequently, the PC linear plot was created based on the scatter plots of each channel. Finally, the PCC was calculated using the following equation:

$$PCC = \frac{\sum ( {R}_{i} - {R}_{av} ) \times ( {G}_{i} - {G}_{av} )}{\sqrt{\sum {( {R}_{i} - {R}_{av} )}^{2} \times \sum {( {G}_{i} - {G}_{av} )}^{2}}} .$$

In the entire image, Rᵢ and Rₐᵥ represent the intensity values and the average intensity of the red channel, respectively, while Gₜ and Gₐᵥ represent the intensity values and the average intensity of the green channel, respectively. PCC ranges from −1 to 1: −1 for a perfect negative correlation (inverse colocalization); 0 for no correlation (no colocalization); 1 for a perfect positive correlation (full colocalization). A higher positive PCC value indicated more colocalization. The SigmaPlot 10.0 was used to generate plots of the analyzed data.

### Exosomal Proteomics

Western blot was carried out as follows: collected exosomes were lysed by adding 1×RIPA buffer, and 20 μg of proteins from whole exosome lysates were separated by Sodium dodecyl-sulfate polyacrylamide gel electrophoresis (SDS-PAGE). The separated proteins were transferred to a nitrocellulose membrane (1662807; Bio-Rad, CA, USA) and treated with primary antibodies (anti-CD9 antibody; anti-CD63 antibody; anti-CD81 antibody; anti-CD9 antibody; anti-neuregulin1 antibody; anti-GalNAc antibody; anti-TGF-beta1 antibody; anti-beta-actin antibody).

Secondary antibodies (anti-mouse HRP-linked antibody; anti-rabbit HRP-linked antibody) were then blotted and immunoreactive species were detected by an ECL substrate using a bioimaging system. Relative expression levels of detected proteins were quantified using ImageJ software.

Sample preparation for MS-based proteomics was as follows: cell-derived exosomes were lysed by 1×RIPA buffer and 50 μg of protein from exosomes was trypsinized and eluted. Eluted peptides were dried down and resuspended, followed by loading in the ultra-high-resolution MS coupled to nano-ultra-high-pressure LC or CE for bottom-up proteomics. The GraphPad Prism 8.0 (GraphPad Software Inc, SD, USA) and SigmaPlot 10.0 were used to generate plots of the analyzed data.

FRET and Inhibition in Membrane Fusion Cells were cultured at 5×10⁴ cells per well of 8-well confocal plates and incubated overnight at 37° C. under 5% CO₂. D-GQD/exo stained with Dil were treated to each well with a concentration of 0.4×10³ exosomes per cell, followed by incubation for 1 h at 37° C. under 5% CO₂. Then, cellular membranes were stained by DiO. Cells were washed, trypsinized, and then collected on a 96-well black plate. Fluorescence intensity was measured before and after cell lysis with Tween-20 using a plate reader. The cell suspension was excited at 484 nm, the DiO excitation wavelength, and the fluorescent emission was measured at 565 nm, the Dil emission wavelength. The SigmaPlot 10.0 was used to generate plots of the analyzed data.

### Example 9

Exosomes from Various Cells of Origin

To investigate the distinct cellular uptake and subsequent cargo release profiles of exosomes from various cells-of-origin, three cell lines were selected for this study: HepG2 cells (human hepatoma), 3T3 cells (mouse fibroblast), and HeLa cells (human cervical adenocarcinoma). Exosomes from culture media of these cell lines were isolated using size-based ultrafiltration techniques. The size and the morphology of exosomes were analyzed using TEM, which confirmed that all isolated exosomes displayed a spherical shape with structural integrity (FIG. 22A). Moreover, based on the quantitative analysis of TEM images, the mean diameters of all three types of exosomes were around 100 nm: 101.3±13.2 nm for HepG2 cell-derived exosomes (HepG2 exo), 99.2±16.8 nm for 3T3 cell-derived exosomes (3T3 exo), and 103.3±15.5 nm for HeLa cell-derived exosomes (HeLa exo). To estimate the concentration and size distribution of exosomes, NTA was conducted on exosome suspension samples (FIG. 22B).

The concentrations of exosomes isolated from 600 mL of serum-free media resulted in 2.80±0.41×10¹⁰ particles/mL for HepG2 exo, 2.62±0.12×10¹⁰ particles/mL for 3T3 exo, and 2.98±0.35×10¹⁰ particles/mL for HeLa exo. A narrow size distribution was obsereved, and the peaks of diameter were 129.7±4.5 nm for HepG2 exo, 130.1±0.4 nm for 3T3 exo, and 136.7±9.7 nm for HeLa exo. The diameters of exosomes measured by NTA appeared to be slightly larger than that of TEM image analysis. This discrepancy was attributed to the specific measurement techniques that were operated in different ways. Using TEM, individual exosome particles were imaged under dehydrated and vacuum conditions, resulting in the observation of shrinkage in exosome size. Moreover, the negative staining conventionally used for TEM imaging samples has been reported to potentially induce distortion and flattening of particle structures, leading to an underestimation of their actual size. The exosomes were measured in solution by NTA that track the individual particles based on their Brownian motion. In NTA, the smaller exosomes may not be detectable due to the limitations of light scattering which led to a shift in the estimated size distribution to a slightly larger range. Therefore, when characterizing the size of exosomes, the disparity in size distribution needs to be taken into consideration and confirmed to ensure that the respective measurements overlap. To evaluate the colloidal stability, the zeta potentials of exosomes from the three cell origins were measured. This was based on the fact that particles with a higher zeta potential display improved stability due to enhanced electrostatic repulsion, effectively hindering aggregation or coagulation. The zeta potentials were measured by Zetasizer: −9.2±1.0 mV for HepG2 exo, −11.5±1.8 mV for 3T3 exo, and −18.8±0.3 mV for HeLa exo (FIG. 22C), proportional to those of their parent cells. Analyzing the NTA data along with zeta potentials, a correlation between the negative charge of exosomes and the characteristics of their size distribution was observed. HeLa exo were found to have the most negative surface charges among the three types of exosomes, and they exhibited a size distribution with a sharp and high peak, suggesting that a significant portion of the exosomes shared a similar size. On the other hand, HepG2 exo had a less negative surface charge compared to the other exosomes, and they exhibited alower and wider distribution of sizes, as indicated by aless pronounced peak in the size distribution. This is attributed to the surface charge that contributes to repulsion between particles, thereby leading to a more dispersed and organized arrangement, resulting in a higher concentration of exosomes around a specific size. Overall, the integrity and colloidal stability of all three types of exosomes were confirmed. Additionally, to further verify that the isolated nanoparticles were indeed exosomes, the expression of exosomal biomarkers was confirmed by conducting a Western blot analysis of CD9, CD63, and CD81, which are commonly enriched in the exosome membrane. The Western blot analysis of exosome lysates detected clear immunoblotted bands for CD9, CD63, and CD81 for all isolated and purified exosomes from the three cell lines (FIG. 22D).

### Example 10

### D-GQDs as Representative Cargos in Exosomes

In this study, chiral GQDs, specifically D-cysteine functionalized GQDs (D-GQDs), were utilized as representative cargo in exosomes (FIG. 23A). The D-GQDs displayed high permeability into exosomes without disrupting the membrane integrity of exosomes due to chiral interaction between D-GQDs and biological lipid membranes. To confirm the permeation of D-GQDs into exosomes, the exosomes were labeled with a red fluorescent membrane dye (PKH26).

Meanwhile, D-GQDs exhibited strong blue fluorescence emission with the maximum emission wavelength at 450-520 nm when excited at 360 nm. These optical properties of D-GQDs allowed the permeation of D-GQDs into exosomes to be observed using CLSM at an excitation wavelength of 405 nm and an emission filter at wavelength of 450 nm. Thus, the colocalization of PHK26-labeled exosomes (red) and D-GQDs (blue) indicated the permeation and accumulation of D-GQDs inside exosomes (FIG. 23B). To ensure the sufficient permeation of D-GQDs as representative cargos into exosomes, the permeation efficiency was quantified using a counting method. It was determined and quantified that D-GQDs (12 μM) were able to permeate into all three types of exosomes (at a concentration of 1×10⁹ particles/mL) with high permeability: 67.2±9.7% for HepG2 exo, 70.6±10.8% for 3T3 exo, and 66.7±4.3% for HeLa exo (FIGS. 23B and 23D), and even achieved 80% by loading with a concentration of 15 μM D-GQDs into the exosomes. These efficiencies were higher than those of any other available drug encapsulation method and thus were sufficient for D-GQDs as representative cargos in additional studies.

Notably, specific points in CLSM images ranging from 100 to 500 nm appeared larger than the actual size of the objects they represent, which are in the range of 30 to 200 nm. This discrepancy can be attributed to diffraction effects and the inherent resolution limitations of the optical microscope, 180 nm laterally and 500 nm axially. The permeation of D-GQDs into exosomes was further quantified using fluorometry. Fluorescence recovery became evident in all three types of exosomes loaded with D-GQDs after lysis with the biological surfactant, Tween-20. This observation indicated that the self-quenching of D-GQDs within the exosomes was alleviated through the lysis of the exosomal membrane, enabling a sufficient distance for them to enhance their fluorescence without being subjected to the quenching effects of neighboring particles in the lysed suspension.

### Example 11

Differential Cellular Uptake of Exosomes Depending on Cell-of-Origin Exosomes stemming from various cell types exhibit significant heterogeneity, profoundly influencing their uptake behavior within recipient cells. To explore the cellular uptake tendencies of exosomes depending on their cell-of-origin and recipient cells, quantitative analysis of CLSM images was conducted. The exosomes were loaded with D-GQDs, enabling the tracking through blue fluorescence, while the recipient cells were stained with cytol-tracible red dye. After hours of co-incubation, the cellular uptake of exosomes into their parental or non-parental cells was imaged using CLSM (FIG. 24). In order to quantitatively analyze the cellular uptake tendencies of exosomes, the distribution of D-GQDs within the recipient cells was examined using the disclosed above equation. In the recipient group of HepG2 cells, the uptake of HepG2 exo was verified to be significantly higher compared to the other two types of exosomes, being ~3.2 times higher than 3T3 exo and ~2.4 times higher than HeLa exo (FIG. 24A). Similarly, in the recipient group of 3T3 cells, the highest uptake of 3T3 exo was observed, which was ~2.2 times higher than and ~1.4 times higher than HepG2 exo and HeLa exo, respectively (FIG. 24B). The consistent trend was also observed in the group of HeLa cells, with uptake of HeLa exo being approximately 2 times higher than HepG2 exo (~1.7 times) and 3T3 exo (~1.9 times) (FIG. 24C).

These results not only agreed with prior findings signifying the propensity of exosomes to home in on their cell-of-origin, but also provided a quantitative comparison of their cellular uptake efficiency in both parental and non-parental recipient cells. In addition to the thorough observation of exosome uptake trends and its quantitative analysis, getting a comprehensive understanding of the intricate mechanisms governing their entry into recipient cells was necessary to reveal the underlying cellular uptake and subsequent mechanisms. In previous studies, the cellular uptake of exosomes has been reported to mainly occur through two primary pathways: endocytic uptake and direct membrane fusion. Different pathways will also lead to distinct cargo release mechanisms: lysosomal entrapment followed by degradation, and direct cargo entry into the cytosol. To further investigate the understanding of distinct uptake efficiency and subsequent cargo release, the potential uptake pathways of exosomes was studied, as well as the processes involved in the delivery of their cargo through tracking D-GQDS, in subsequent experiments.

### Example 12

### Endocytosis via Cell Receptors-Exosome Ligands Interaction

To investigate the endocytic pathways involved in the uptake of exosomes by their parental/non-parental cells, as well as the releasing sites of their cargo, in-depth cellular imaging was conducted using CLSM. LysoView dye was used to stain the lysosomes of the recipient cells. This enabled the observation of how exosomes, upon entering the endocytic pathway, become trapped in endosomes, eventually leading to their cargos entrapped in lysosomes. Exosomes and cells were incubated for 4 h to ensure a sufficient uptake of exosomes by the cells through the endocytic process. The colocalized spots of lysosomes and D-GQDs were quantified to analyze the entrapment of exosomes within lysosomes in recipient cells. Remarkably, the results demonstrated that the amount of intraspecies exosomes entrapped within lysosomes were significantly higher than cross-species exosomes (FIG. 25A). HepG2 exo was found to be entrapped within lysosomes of HepG2 cells ~2.1 times more than 3T3 exo and ~1.9 times more than HeLa exo (FIG. 25B). Similarly, in HeLa cells, the number of colocalization spots for HeLa exo was significantly higher, approximately 2 times higher than HepG2 exo (~1.7 times) and 3T3 exo (~2 times) (FIG. 25D). Although the colocalization in 3T3 cells was relatively low compared to the other two cancer cell lines, HepG2 and HeLa cells, the consistent trend was observed. 3T3 exo still exhibited colocalization with lysosomes more than ~1.6 times than HepG2 exo and HeLa exo (FIG. 25C). These results indicated that while intraspecies exosomes are preferentially taken up into their parental cells, a large number of exosomes were involved in endocytic uptake, leading to their entrapment in lysosomes.

Endocytosis is primarily mediated by receptor-ligand interactions. To identify the specific ligands involved in the endocytic uptake pathway with parental cells, further investigations were conducted. By comparing the analysis of the surface protein composition of exosomes using Mass Spectrometry (MS), the presence of distinct proteins on three different exosomes were identified and confirmed that played a crucial role in mediating receptor-ligand interactions with their parental cells, facilitating the endocytic uptake (FIG. 26A). Based on the analysis of MS-based proteomics data, several protein accessions were classified into eight distinct proteins: 5 protein accessions for Transforming growth factor-beta 1 (TGF-β1), 13 protein accessions for Glycoproteins (without or with N-acetylgalactosamine (GalNAc) residues, 8 protein accessions and 5 protein accession, respectively), 1 protein accession for Neuregulin 1 (NRG1), 1 protein accession for Beta-actin (s-actin), 3 protein accessions for Clathrin Heavy Chain 1 (CHC1), 4 protein accessions for Heat Shock Protein 70 (HSP70), 6 protein accessions for Heat Shock Protein 90 (HSP90), and 1 protein accession for vinculin (FIG. 26A). TGF-β1 is a versatile cytokine that regulates various cellular functions, including a wide range of growth factors that control cell growth, differentiation, and other essential cellular processes. When TGF-β ligands bind to the TGF-β receptor complex, consisting of type I TGF-β receptor (TβRI) and type II TGF-β receptor (TβRII), it triggers a series of endocytic uptake processes. Glycoproteins are proteins with attached carbohydrate molecules found in living systems, including cell membranes and they play vital roles in cellular functions. According to the previous reports, glycoproteins with terminal galactose (Gal) and/or N-acetylgalactosamine (GalNAc) residues exhibit high specificity and affinity for the liver-specific Asialoglycoprotein receptor (ASGPR), leading to their internalization through endocytosis. Specifically, in HepG2 cells, a significant overexpression of ASGPR on the cell membranes was demonstrated, whereas notably lower ASGPR expression was observed on HeLa cells. NRG1 is a signaling protein that regulates cell-to-cell communication and plays critical roles in cell function. NRG binds to the Epidermal Growth Factor Receptor (EGFR), which includes family members ErbB1, ErbB2, ErbB3, and ErbB4, followed by subsequent cellular processes such as endocytosis. In particular, it was reported that NRG1 directly binds to ErbB3 and ErbB4, but does not bind directly to ErbB2, but is related functionally to ErbB2. EGFR is reported to be overexpressed in around 90% of cervical tumors, including HeLa cells.

Furthermore, ErbB4 was commonly observed on the surface of HeLa cells. The interaction between specific ligands (TGF-β1, GalNAc, and NRG1) with their receptors (TβR, ASGPR, and ErbB4) were demonstrated respectively to be followed by subsequent cellular processes, such as clathrin-dependent endocytosis. This receptor-mediated endocytosis involves clathrin-coated pits and vesicles assembled and formed by CHC. The detection of CHC1 in all three exosomes provided additional evidence supporting the internalization of specific molecules from the extracellular space through endocytosis. Both HSP70 and HSP90 have been commonly found associated with exosomes, identifying them as exosomal markers. β-actin and Vinculin exhibit stable expression in most cell types and are commonly used as loading controls to normalize protein expression across samples. The presence of these proteins detected in all exosome samples confirmed the reliable measurement of the MS-based proteomics data.

To confirm the expression levels of TGF-β1, GalNAc, and NRG1, which are closely related to the interaction with their parental recipient cells, Western blot analysis was performed (FIG. 26B). In order to ensure the accuracy and reliability of quantitative analysis, s-actin was used as the loading control, and the relative expression levels were normalized using β-actin. The expression level of TGF-β1 on HeLa exo was found to be two-fold higher than that of HepG2 exo at a blotting concentration of 0.3 μg/mL of anti-TGF-β1 antibodies, while no band was observed in 3T3 exo (FIG. 26C). According to previous reports, both HepG2 and HeLa cells exhibit a high expression level of TβRII, and significant responsiveness to TGF-β1. Hence, when HepG2 exo and HeLa exo are exposed to cell surfaces expressing TOR, they are likely to undergo the receptor-ligand interaction-mediated endocytic process. Notably, intraspecies exosome groups were observed to be prominently entrapped within lysosomes in both HepG2 and HeLa cells (FIGS. 25B and 25D). In contrast, no significant entrapment was observed in the intraspecies group in 3T3 cells (FIG. 25C). These results were attributed to the interaction between TGF-β1 expressed on exosomes and TOR present in HepG2 cells and HeLa cells. Moreover, the expression of glycoproteins with GalNAc residues on HepG2 exo was observed, while no expression in 3T3 exo and HeLa exo was observed at a blotting concentration of 0.4 μg/mL anti-GalNAc antibodies (FIG. 26D). This implies that significant endocytosis will take place through the interaction between GalNAc on HepG2 exo and the overexpressed ASGPR on HepG2 cells. Indeed, the predominant lysosomal uptake of HepG2 exo by HepG2 cells was observed (FIG. 25B). Furthermore, consistent with the MS-based proteomics, the expression of NRG1 was detected only in HeLa exo at a blotting concentration of 0.4 μg/mL of anti-TGF-β1 antibodies, while no expression was observed in 3T3 exo and HepG2 exo (FIG. 26E). Based on the comprehensive analysis of lysosomal uptake results in HeLa cells, the interaction between NRG1 on HeLa exo and ErbB4 on HeLa cells was confirmed that led to a significant increase in the lysosomal entrapment of HeLa exo compared to HepG2 exo and 3T3 exo (FIG. 25D). These experiments illustrated that the intraspecies exosomes carry ligands (TGF-β1, GalNAc, and NRG1) capable of interacting with receptors (TOR, ASGPR, and ErbB4) on their parental cells, resulting in endocytic uptake followed by the entrapment of exosomal cargo within lysosomes.

### Example 13

Cellular Uptake Via Direct Membrane Fusion with Exosomes

Direct membrane fusion is another primary pathway for cellular uptake of exosomes. To further investigate the underlying mechanism of membrane fusion, colocalization examination and Fluorescence Resonance Energy Transfer (FRET) analysis were performed. First, a red membrane fluorescent dye, Dil, was introduced to label the lipid bilayer of exosomes. After incubation with exosomes for 1 h, the cells were stained with a green membrane fluorescent dye, DiO. As shown in CLSM images (FIG. 27A-C), a significant overlap of cellular membranes (green) and exosomal membranes (red) was observed in the groups treated with cross-species exosomes, indicating the exosome fused with cell membrane. For the quantitative analysis, the obtained CLSM images were compared using Pearson's correlation coefficient (PCC), which is a statistical measure in colocalization analysis, quantifying pixel intensity similarity in two fluorescence channels to indicate colocalization extent. Higher PCC value indicated more colocalization between the two channel signals: green, representing cellular membranes, and red, representing exosomal membranes. In HepG2 cells, the PCC value for HepG2 exo was slightly higher than that of the negative control that was treated with media only. In contrast, the PCC values for 3T3 exo and HeLa exo were estimated to be over two-fold higher than that of HepG2 exo (FIG. 27A). In the same way, in HeLa cells, the control and HeLa exo groups showed only a small correlation value less than 0.4, whereas both cross-species exosomes, HepG2 and 3T3, estimated a strong correlation with the large PCC (>0.7) (FIG. 27C). Consistently, a parallel trend in the 3T3 cell group was observed, although the difference in colocalization between intraspecies and cross-species exosomes was less pronounced compared to HepG2 and HeLa cells. Both HepG2 exo and HeLa exo exhibited a strong correlation between the red and green channels, with PCC values greater than 0.7. On the other hand, 3T3 exo showed a moderate correlation value, ranging from 0.4 to 0.7 (FIG. 27B). These findings validated that the PCC of cross-species exosomes mostly exhibited large correlation values, indicating a high degree of colocalization between two fluorescent membranes. This observation suggested that the uptake pathway of cross-species exosomes was more likely to involve direct membrane fusion. Additionally, the presence of exosomes (red dots) within intracellular area was clearly visible in the groups treated with intraspecies exosomes across all three cell types. This observation suggested endocytic uptake occurred even after just 1 hour of incubation (FIG. 27A-C). These findings provided further evidence that intraspecies exosomes were internalized more effectively through the endocytic pathway (FIG. 25).

In addition, to confirm that the overlapping of the green and red signals of CLSM images indeed resulted from membrane fusion, FRET analysis was conducted. FRET provides valuable evidence of the proximity and interaction of fluorescent molecules, as it enables detection of the energy transfer between a donor and acceptor fluorophore within a range of 1-10 nm. In this experiment, DiO was used as the fluorescence donor, and Dil was used as the fluorescent acceptor to measure FRET. In the intraspecies exosome-treated groups, either a minimal or no FRET-mediated fluorescence signal was observed in the CLSM images. On the other hand, in the cross-species exosome treated group, the CLSM images clearly showed significant red fluorescence (represented in gray), which indicated notable membrane fusion (FIG. 27A-C). These FRET signals generated from membrane fusion were further validated by measuring fluorescence intensity (FI) using a plate reader. After 1 h incubation, the cell suspension was excited at 484 nm, which corresponded to the DiO excitation wavelength, and the emitted fluorescence was measured at 565 nm, which corresponded to the Dil emission wavelength. The cross-species exosomes exhibited significantly higher FI in FRET, whereas intraspecies exosomes mostly exhibited lower FI in FRET, comparable to the control group or distinctly lower than cross-species exosomes (FIG. 27A-C). Afterwards, the inhibition of FRET was also carried out by lysing cells and exosomes with Tween-20, and the fluorescence intensity change in the “After lysis” groups was confirmed (FIG. 27A-C). The elimination of quenching resulted in an increase in FI for the control and intraspecies exosome group. However, the FRET generated from cross-species membrane fusion was inhibited, leading to a decrease in FI. The effective inhibition of FRET was further ensured by observing larger error bars in the “After lysis” groups compared to the “Before lysis” groups. These results were coherently supported by the analysis of PCC values. Overall, both CLSM analysis and fluorimetry demonstrated the propensity for membrane fusion in cross-species exosomes.

### Example 14

### Different Exosomal Cargo Release is Dependent on Uptake Pathways

To trace exosomal cargo, the retention and release of D-GQDs in/from exosomes was observed using CLSM when they were taken up by parental/non-parental recipient cells. D-GQDs were loaded into different exosomes to represent exosomal cargo, and the exosomal membranes were stained with PKH26. After 6 h of incubation with cells, the cells were stained with a green cytoplasmic membrane dye, enabling the selective imaging of cell membrane boundaries. D-GQDs loaded in intraspecies exosomes were seen colocalizing as purple dots within the cells (FIG. 28), implying that exosomes were taken up by parental recipient cells while preserving their structural integrity. This suggested that the cellular uptake of intraspecies exosomes occurred through endocytosis, resulting in the entrapment of exosomes within lysosomes and intercepting the release of cargo. On the other hand, D-GQDs loaded in cross-species exosomes were observed escaping from the exosomal membrane, represented by the arrows in FIG. 28. Upon the fusion of exosomal membranes with cellular membranes, the immediate release of exosomal cargo was observed without the exosomes being enclosed in other lipid vesicles. This fusion process involves a hemifusion stage, followed by the formation of a fusion pore that facilitates the direct transfer of cargo between exosomes and cells. From these CLSM images, distinct trends in cargo release were observed that correlate with specific uptake pathways, aligning with the lysosomal uptake trends (FIG. 25) and the membrane fusion trends (FIG. 27). Comprehensively, it was demonstrated that intraspecies exosomes preferentially undergo uptake by their parental recipient cells through endocytosis, leading to cargo entrapment within lysosomes (FIG. 29A), whereas cross-species exosomes are more inclined to directly release their cargo through membrane fusions to non-parental recipient cells (FIG. 29B).

While the observation of cellular uptake trends of exosomes involving the endocytic pathway and direct membrane fusion has been explored, the detailed mechanisms governing their entry into both parental and non-parental recipient cells are not yet fully understood. Several approaches have been developed to trace exosomes and their cargo. However, these approaches have some challenges in effectively tracking the journey of exosomes from extracellular interactions to the intracellular release of their cargo. This limitation arises due to inaccuracies resulting from uneven or non-specific staining, coupled with the potential of dyes to modify the cellular membrane properties of exosomes. On top of that, while loading traceable probes into exosomes is of utmost importance to explore intracellular cargo release tracking, the conventional cargo-probe loading approaches face challenges due to their low loading efficiency and the potential risk of damaging the exosomal membrane. Therefore, the lack of comprehension regarding their physiological tendencies, which are involved in their interactions with recipient cells and the intracellular release of their cargo depending on the uptake pathways, leads to a challenge in the effective application of exosomes for drug delivery in clinical uses. In this study, chiral GQDs were utilized to track exosomal cargo, taking advantage of their effective membrane permeability and optical properties while maintaining the structural integrity of the exosomal membrane. By loading D-GQDs into distinct exosomes having different lipid and protein contents and originating from three distinct cell types, a better understanding of the mechanisms of exosomal uptake was gained. This process is significantly dependent on both the cell-of-origin and the recipient cell types. Additionally, further investigations into the subsequent tendency of cargo release based on the specific uptake pathways were conducted.

The CLSM results generated by analyzing the uptake profile of D-GOD-loaded exosomes confirmed that intraspecies exosomes are preferentially taken up by their corresponding parental cells compared to cross-species exosomes (FIG. 24). It was further revealed that the enhanced uptake of intraspecies exosomes results from a higher proportion of endocytic uptake facilitated by ligand-receptor interactions (FIG. 25-26). Notably, in the HeLa cell group, HepG2 exo exhibited a markedly higher lysosomal uptake compared to 3T3 exo, a difference that was statistically significant with a p-value of less than 0.05 (FIG. 25D). However, in the HepG2 cell group, HeLa exo did not exhibit a significant difference from 3T3 exo (FIG. 25B). These observations implied that HepG2 exo had a greater propensity to interact with membrane surface proteins, potentially TβR, on HeLa cells, than HeLa exo did on HepG2 cells. It has been reported that the cleavage of exosome surface proteins results in a reduction of their association with recipient cells, confirming the significance of specific proteins that facilitate the cellular uptake of exosomes through an endocytic uptake pathway.

A large number of intraspecies exosomes was found to be present within lysosomes, rather than their cargo being released into the cytosolic area (FIG. 25). Despite achieving high exosome uptake into their parent cells mainly through receptor-ligand mediated endocytosis, it is necessary to take into account the entrapment of exosomes within endosomes, along with their potential subsequent processes of maturation into lysosomes and degradation. Even though endocytosis allows effective cellular uptake of exosomes, this process leads to their subsequent processing and degradation in endosomes and lysosomes, where an acidic environment facilitates various enzymatic activities and is replete with hydrolytic enzymes. To ameliorate or circumvent undesired degradation and deactivation of therapeutic efficacy within the lysosome, it is necessary to ensure effective drug delivery to intracellular sites without degradation through successful endosome/lysosome escape of exosomes. However, it has previously been reported that only 24±1.2% of intake exosomes were released from endosome/lysosome. This highlights one of the challenges faced in utilizing pristine exosomes for drug delivery. Therefore, a functionalization approach, such as surface modification on exosomes to facilitate endosomal/lysosomal escape, holds significant potential for advancing therapeutic exosomes. This strategy enables the full utilization of the inherent homing effect of exosomes for targeted drug delivery.

Another pathway of exosome uptake is direct membrane fusion with the plasma membrane of recipient cells. Cross-species exosomes were found to exhibit a pronounced membrane fusion, whereas the membrane fusion of intraspecies exosomes was less observed (FIG. 27). When cross-species exosomes were incubated with recipient cells, a strong correlation with PCC values larger than 0.7 was observed. In contrast, a weak correlation with PCC values, mostly smaller than 0.4, was seen in the intraspecies exosome-treated groups (FIG. 27A-C). Interestingly, the 3T3 cell group exhibited a relatively higher PCC (moderate PCC values of 0.4~0.7) when incubated with intraspecies exosomes, compared to the HepG2 and HeLa cell groups (FIG. 27B). Combined with the analysis of overall lower lysosomal uptake counts in 3T3 cells (FIG. 25C), these results suggested that despite that endocytosis mediated by the receptor-ligand interaction remains dominant for intraspecies uptake of exosomes, non-cancerous cells, such as 3T3 cells, are more likely to take up 3T3 exo through membrane fusion compared to cancerous cells, such as HepG2 cells and HeLa cells. Direct membrane fusion between lipid membranes is a highly regulated and orchestrated process involving several factors. The lipid composition of both exosomes and the cell membrane plays an important role in fusion. H⁺ associated lipids, such as phosphatidylethanolamine (PE) and phosphatidylserine (PS), were previously reported to facilitate monovalent cation-induced fusion. In certain lipid compositions, changes in calcium levels and pH can trigger membrane fusion by destabilizing the lipid bilayers, thereby promoting the fusion process. The presence of specific proteins known as fusogens, such as SNARE proteins, also influences membrane fusion.

In this study, fluorescent chiral GQDs were utilized to demonstrate not only the preferential uptake pathway of exosomes, depending on their cell-of-origin and recipient cell type, but also the cargo release associated with the specific uptake pathway. These chiral GQDs were derived by surface functionalization with the chiral ligand D-cysteine (D-GQDs), exhibiting high permeability into exosomes through nanoscale chirality matching and interaction with lipid membranes. Importantly, the size and morphology of exosomes remained intact after the successful loading of D-GQDs. In addition, chiral GQDs exhibit unique optical properties, particularly their fluorescence emission, allowing for their application in bioimaging and biosensing. Intraspecies exosomes were found to mainly be taken up by their parental recipient cells through receptor-ligand interaction-mediated endocytosis, leading to cargo entrapment within lysosomes. On the other hand, despite the lower uptake efficiency, cross-species exosomes were predominantly taken up by non-parental recipient cells through direct membrane fusion, followed by direct cargo release into the cytosol. This study provides important insights that contribute to the progress of effectively utilizing lipid-based carriers as drug delivery vehicles and enhance the understanding of intercellular communications.

### Example 15

### Tau-Specific N-Amino Peptide (NAP)-Chiral Graphene Quantum Dot-Loaded Exosomes for Tauopathy Therapy

N-amino peptides (NAPs) are highly specific ligands for targeting tau protein aggregation, which is a key factor in neurodegenerative diseases. The structure of NAPs includes soluble peptidomimetics that feature amide substitutions, which are important for their stability and functionality. One example of a specific NAP called “Qal” was developed having the amino acid sequence of CGTHKLTFRASHAVQIVYKGC (SEQ ID NO: 2) (FIG. 31, top). These peptides incorporate the aggregation-prone hexapeptide motif PHF6, which is directly involved in the pathological aggregation of tau proteins. The design of NAPs allows them to specifically target and inhibit tau aggregation, a pivotal process in the progression of disorders such as Alzheimer's disease. This selectivity is important for ensuring that the treatment targets only the pathological tau aggregates, reducing the likelihood of off-target effects and increasing the potential for therapeutic application in neurodegenerative diseases. However, the translational use of NAPs in the clinic is limited due to low bioavailability intracellularly. On the other hand, chiral GQDs with engineered properties (FIG. 31, bottom) are a promising and effective platform to inhibit and disassemble pathological aggregation of tau proteins. As described herein, the unique features of NAPs and chiral GQDs were utilized to design an exosome-based delivery strategy to enhance the specificity and efficacy of tau-targeting therapeutics (FIG. 30).

NAPs were found to form a stable complex with D-cys-GQDs via pi stacking, and this complex could be loaded into exosomes with high efficiency (FIG. 32-33). The loading efficiency of Qal on D-GQDs was determined by fluorescence quenching assay. Different concentrations of D-GQDs (0, 0.5, 1, 1.5, 2.5, 3.5, 4.5, 6, 8, and 10 μM) were incubated with 10 μM of Qal and the fluorescence of Qal from 295 nm to 400 nm was significantly quenched with the increase of D-GQD concentration (FIG. 32A-B). The quenching efficiency of Qal began to reach a plateau when the concentration of D-GQDs was higher than 3.5 μM (FIG. 32B). Based on the molar concentrations of Qal and D-GQDs at the plateau of quenching efficiency, each D-GQD was able to carry 3 Qal molecules. The fluorescence change of D-GQD-loaded exosomes (3T3-Exo) at each washing step was monitored. The maximum emission wavelength of both the exosome complex solution and the eluent was around 425 nm after washing six times, which was attributed to the background fluorescence of pure 3T3-Exo and PBS. These results suggest that unincorporated free D-GQDs can be removed after washing six times with PBS (FIG. 33A-B).

The permeation of Qal-bound D-GQDs into exosomes (3T3-Exo) was detected by the accumulation of intrinsic blue fluorescence of D-GQDs (around 450 nm) in the exosomes using a confocal microscope (FIG. 34). The permeation efficiency of D-GQDs into exosomes was quantified using a previously developed method. Briefly, the permeation efficiency of D-GQDs into exosomes was determined by statistically analyzing the total number of D-GQD-loaded exosomes as blue dots in confocal images over the total number of exosomes obtained from nanoparticle tracking analysis (NTA). The permeation efficiency of Qal-bound D-GQDs (15 μM) into 3T3-Exo (at a concentration of 1×10⁹ particles/mL) was determined to be 62.6±6.5% (FIG. 34).

To study the effect of Qal-D-GQD-Exo on tau propagation, a tau biosensor seeding assay (FIG. 35) was used to investigate whether the Qal-D-GQD-Exo could block the cellular transmission of exogeneous mature tau fibrils using three different ratios of Qal to D-GQDs (10:1, 6:1, and 3:1) and concentrations of D-GQDs at 0.8, 0.4, 0.2, and 0.1 μM. The results showed that the inhibition ability of Qal-D-GQD-Exo and D-GQD-Exo on the seeding capacity of tau fibrils was concentration-dependent, and that Qal-D-GQD-Exo inhibited at most about 70% of intracellular tau aggregation, as compared to at most about 60% inhibition for D-GQD-Exo (FIG. 36). Last, a bare Qal control treatment group did not show any inhibitory efficiency in the experiment (FIG. 37), indicating that loading Qal into exosomes using D-GQDs can increase tau inhibition of D-GQD-Exo by specifically targeting D-GQDs to tau intracellularly. These results demonstrate that the chiral GQDs can be effectively and efficiently loaded with various types of drug cargo including peptides such as NAPs.

What is claimed:

1. A composition for enhanced drug loading into a lipid-based carrier, the composition comprising:
a chiral graphene quantum dot comprising a graphene quantum dot functionalized with a non-aromatic chiral ligand having a single chiral center, wherein a chirality of the chiral graphene quantum dot matches a chirality of a lipid content of the lipid-based carrier; and
a drug bound to the chiral graphene quantum dot.

2. The composition of claim 1, wherein the graphene quantum dot has a size of about 1 nm to about 15 nm.

3. The composition of claim 1, wherein the graphene quantum dot is functionalized with the non-aromatic chiral ligand through covalent or noncovalent conjugation.

4. The composition of claim 1, wherein the graphene quantum dot is functionalized with about 1 to about 10 molecules of the non-aromatic chiral ligand.

5. The composition of claim 1, wherein the non-aromatic chiral ligand is negatively charged or positively charged at physiological conditions.

6. The composition of claim 1, wherein the non-aromatic chiral ligand comprises a D-amino acid or a L-amino acid.

7. The composition of claim 1, wherein the chiral graphene quantum dot has a zeta potential of about −4 mV to about 4 mV.

8. The composition of claim 1, wherein the chiral graphene quantum dot has a two-dimensional nanosheet structure having left- or right-handed twists characterized by a dihedral angle formed from an outer edge of the chiral graphene quantum dot to a center of the chiral graphene quantum dot, the dihedral angle ranging from about 0° to about 45°.

9. The composition of claim 1, wherein the drug is bound to the chiral graphene quantum dot through π-π stacking, van der Waals interactions, hydrophobic interactions, covalent bond interactions, electrostatic interactions, or combinations thereof.

10. The composition of claim 1, wherein the drug comprises a hydrophobic drug, a hydrophilic drug, or an amphiphilic drug.

11. The composition of claim 1, wherein the drug comprises a nucleic acid, a peptide, a polypeptide, an antibody, a small molecule, or a combination thereof.

12. The composition of claim 11, wherein the drug is a nucleic acid selected from the group consisting of a siRNA, a shRNA, and an antisense oligonucleotide.

13. The composition of claim 1, wherein about 1 to about 30 molecules of the drug are bound to the chiral graphene quantum dot.

14. The composition of claim 1, wherein the lipid content of the lipid-based carrier comprises phosphatidylserines, phosphatidylcholines, phosphatidylinositols, phosphatidylethanolamines, phosphatidylglycerols, sphingomyelins, cholesterols, glycosphingolipids, or combinations thereof.

15. A method for enhanced drug loading into a lipid-based carrier, the method comprising:
binding a drug to a chiral graphene quantum dot comprising a graphene quantum dot functionalized with a non-aromatic chiral ligand having a single chiral center to generate a drug-bound chiral graphene quantum dot, wherein a chirality of the chiral graphene quantum dot matches a chirality of a lipid content of the lipid-based carrier; and
combining the drug-bound chiral graphene quantum dot with the lipid-based carrier to load the drug into the lipid-based carrier.

16. The method of claim 15, wherein the lipid-based carrier comprises an extracellular vesicle (EV), a small extracellular vesicle (sEV), an exosome, an ectosome, a microvesicle, a liposome, a lipoprotein, a lipid nanoparticle, an exomere, a supermere, or combinations thereof.

17. The method of claim 15, wherein the lipid-based carrier has a size of about 10 nm to about 500 nm.

18. The method of claim 15, wherein the lipid content of the lipid-based carrier comprises phosphatidylserines, phosphatidylcholines, phosphatidylinositols, phosphatidylethanolamines, phosphatidylglycerols, sphingomyelins, cholesterols, glycosphingolipids, or combinations thereof.

19. The method of claim 15, wherein about 1.0×10⁴ to about 1.0×10⁶ molecules of the drug-bound chiral graphene quantum dot are combined with a single molecule of the lipid-based carrier.

20. The method of claim 15, wherein the drug-bound chiral graphene quantum dot is incubated with the lipid-based carrier for about 1 min to about 30 min at about 20° C. to about 40° C. to load the drug into the lipid-based carrier.

21. The method of claim 15, wherein the drug-bound chiral graphene quantum dot permeates into the lipid-based carrier via matching of the chirality of the chiral graphene quantum dot to the chirality of the lipid content of the lipid-based carrier to load the drug into the lipid-based carrier.

22. The method of claim 15, wherein the method achieves a drug loading efficiency of greater than 60% into the lipid-based carrier.

23. A drug-loaded lipid-based carrier generated by the method of claim 15.
